(19) United States
(12) Patent Application Publication
(10) Pub. No.: US 2020/0095646 A1
(43) Pub. Date: Mar. 26, 2020

(54) HIV-1 INCIDENCE BIOMARKERS

(71) Applicant: Duke University, Durham, NC (US)

(72) Inventors: Georgia Tomaras, Durham, NC (US); Kelly Seaton, Durham, NC (US); Xiaoying Shen, Durham, NC (US); Nicole Yates, Durham, NC (US); Hua-xin Liao, Durham, NC (US); Barton F. Haynes, Durham, NC (US); Nathan Vandergrift, Durham, NC (US); Wes Rountree, Durham, NC (US); John Bainbridge, Durham, NC (US)

(21) Appl. No.: 16/315,688

(22) PCT Filed: Jul. 5, 2017
(86) PCT No.: PCT/US17/40744

§ 371 Date: Jan. 7, 2019

Related U.S. Application Data
(60) Provisional application No. 62/358,477, filed on Jul. 5, 2016.
(60) Provisional application No. 62/358,983, filed on Jul. 6, 2016.
(60) Provisional application No. 62/427,607, filed on Nov. 29, 2016.
(60) Provisional application No. 62/456,885, filed on Feb. 9, 2017.

(51) Int. Cl. C12Q 1/70; C07K 14/16

(52) U.S. Cl. CPC C12Q 1/703; C07K 14/162

(57) ABSTRACT

The invention is directed to methods, reagents and kits for detecting incident HIV-1 infection.

This application claims priority from U.S. Provisional Application No. 62/358,477, filed on Jul. 5, 2016, U.S. Provisional Application No. 62/358,983, filed on Jul. 6, 2016, U.S. Provisional Application No. 62/427,607, filed on Nov. 29, 2016, and U.S. Provisional Application No. 62/456,885, filed on Feb. 9, 2017, the entire contents of each which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The invention is directed to methods and reagents for detection of incidence of HIV-1 infection.

### BACKGROUND OF THE INVENTION

HIV-1 Incidence measures the rate of new infections in specific populations; and thus provides information on the nature of the epidemic and the effectiveness of prevention strategies. Limitations of the current technology include misclassification of recent infection vs. chronic infection, particularly across HIV-1 clades and a lack of sensitivity of current incidence tests. (2)

### SUMMARY OF THE INVENTION

In some aspects the invention provides a set of four antigen/antibody biomarkers, that are derived from a Discriminant Function Analysis of a training sample set, that can be used in a method to classify samples, of known or unknown status, as recent or long standing infection. In one embodiment, the set comprises four antigen/antibody biomarkers. In one embodiment, the set comprises four antigen/antibody biomarkers selected from the list of antigen-antibody pairs listed in FIG. 61 or FIG. 62.

In one aspect the invention provides a method for classifying HIV-1 status of a sample from a subject as recent or long standing. In some embodiments, the method comprises:
a. Measuring in the sample binding of IgG3 antibodies to Clade C gp140 envelope (measurement X1),
b. Measuring in the sample the avidity of IgG4 antibodies binding to T/F Clade C gp140 envelope (measurement X2),
c. Measuring in the sample the avidity of IgG4 antibodies binding to Clade B gp140 envelope (measurement X3),
d. Measuring in the sample the avidity of IgG to gp41 immunodomninant domain (ID) epitope (measurement X4), wherein the avidity of IgG gp41 ID epitope is determined as difference between IgG Citrate gp41 ID epitope and IgG PBS gp41 ID epitope,
e. Using measurements X1, X2, X3 and X4 to determine recent (R) or longstanding (L) discriminant value (DVⱼ is used interchangeably Cⱼ), wherein “j” is recent (R) or longstanding (L) class, wherein cut off period for R is nine months, and wherein
   i. DV_{R}=co_{R}+c_{1R}X1+c_{2R}X2+c_{3R}X3+c_{4R}X4, wherein in non-limiting embodiments R classification coefficients are as follows: Table 40 or Table 41, and
   ii. DV_{L}=co_{L}+c_{1L}X1+c_{2L}X2+c_{3L}X3+c_{4L}X4, wherein in non-limiting embodiments L classification coefficients are as follows: Table 40 or Table 41; and
   wherein if DV_{R} is larger than DV_{L}, the sample is classified as Recent, or
   wherein if DV_{L} is larger than DV_{R} the sample is classified as Longstanding.

In one aspect the invention provides a method for classifying HIV-1 status of a sample from a subject as recent or long standing, the method comprising: detecting and quantifying the formation of antibody-antigen complexes, wherein the antibodies-antigen complexes are binding of IgG3 antibodies to Clade C gp140 envelope (measurement X1), avidity of IgG4 antibodies binding to T/F Clade C gp140 envelope (measurement X2), avidity of IgG4 antibodies binding to Clade B gp140 envelope (measurement X3), avidity of IgG to gp41 immunodomninant domain (IG) (measurement X4), wherein the avidity of IgG gp41 ID is determined as difference between IgG Citrate gp41 ID epitope and IgG PBS gp41 ID epitope, using measurements X1, X2, X3 and X4 to determine recent (R) or longstanding (L) discriminant value (DVⱼ is used interchangeably Cⱼ), wherein “j” is recent (R) or longstanding (L) class, wherein cut off period for R is nine months, and wherein
i. DV_{R}=co_{R}+c_{1R}X1+c_{2R}X2+c_{3R}X3+c_{4R}X4, wherein in non-limiting embodiments R classification coefficients are as follows: Table 40 or Table 41, and
ii. DV_{L}=co_{L}+c_{1L}X1+c_{2L}X2+c_{3L}X3+c_{4L}X4, wherein in non-limiting embodiments L classification coefficients are as follows: Table 40 or Table 41; and wherein if DV_{R} is larger than DV_{L}, the sample is classified as Recent, or wherein if DV_{L} is larger than DV_{R} the sample is classified as Longstanding.

In some embodiments, the DVj (Cj) equation is used to calculate the probability of a sample being classified as recent or longstanding (Crecent or Clongstanding). Without being bound by theory, for a given data set comprising DVj scores from multiple samples, the posterior probabilities (PP) are used to determine thresholds for recent or longstanding classification, and can be adjusted to fine tune MDRI and FRR. For example, a PP=0.5 indicates a threshold where a sample with a probability Crecent>0.5 would be classified as recent. A PP=0.4 indicates a threshold where a sample with probability Crecent>0.4 would be classified as recent. In general, lowering the PP threshold for recency lengthens the MDRI. In some embodiments, measurements for a plurality of antigen-antibody pairs is conducted in a single reaction.

In some embodiments, the methods are applied to a plurality of samples comprised in a data set(s), wherein the methods further comprise determining MDRI, FRR, or both, wherein the MDRI and FRR could be determined using different PP thresholds.

Any suitable method could be used to detect and quantify the formation of antibody-antigen complexes, and/or to measure avidity of antibody-antigen interactions.

In some embodiments the methods are used retrospectively to analyze and classify samples. In some embodiments, the methods are used prospectively to analyze and classify samples.

In some embodiments, the methods are used for national surveillance; program, prevention or trial planning; key or sentinel populations; impact assessment; care-based surveillance; research purposes; individual patient management; and/or targeted prevention planning (see FIG. 66). For example, the methods can be used for identification of patients with recent infection or longstanding infection so to guide clinical management and/or public health programs (e.g., selecting therapy, and/or prioritizing contact tracing).

In some embodiments, Clade C gp140 envelope for measurement X1 is any one Clade C envelope in FIG. 62. In some embodiments, Clade C gp140 envelope for measurement X1 is BF1266 gp140 (FIG. 59), for example, comprising LANL database accessioning number HM215360. In some embodiments, T/F Clade C gp140 envelope for measurement X2 is any one of the T/F clade C envelopes in FIG. 62. In some embodiments, the T/F clade C gp140 for measurement X2 is T/F CH505 gp140 (FIG. 59, for example, comprising LANL database accessioning number KC247557. In some embodiments, the clade B gp140 envelope for measurement X3 is any one of Clade B envelopes in FIG. 62, for example comprising LANL database accessioning number AY835447, AY835441, or AY835451. In some embodiments, the clade B gp140 envelope for measurement X3 is SC42261 gp140 (FIG. 59), for example comprising LANL database accessioning number AY835441. In some embodiments, the gp41 ID epitope for measurement X4 is any one listed in FIG. 62. In some embodiments, the gp41 ID epitope for measurement X4 is B.con03 B.con03 ID/(Bio-GGG-BC.con03 ID), comprising amino acid sequence: Bio-GGG-KQLQARVLAVERYLKDQQLLGIWGCSGKLICTTAV.

In some embodiments of the methods the Clade C gp140 envelope for measurement X1 is BF1266 gp140 (FIG. 59), the T/C clade C gp140 for measurement X2 is T/F CH505 gp140 (FIG. 59), the clade B gp140 envelope for measurement X3 is SC42261 gp140 (FIG. 59) and the gp41 ID epitope for measurement X4 is B.con03 ID/(Bio-GGG-BC.con03 ID). In some embodiments,
i. For DV_{R}=co_{R}+c_{1R}X1+c_{2R}X2+c_{3R}X3+c_{4R}X4, the R classification coefficients are as follows: Table 40, and
ii. For DV_{L}=co_{L}+c_{1L}X1+c_{2L}X2+c_{3L}X3+c_{4L}X4, the L classification coefficients are as follows: Table 40; and wherein if DV_{R} is larger than DV_{L}, the sample is classified as Recent, or wherein if DV_{L} is larger than DV_{R} the sample is classified as Longstanding.

In some aspects the invention provides methods for classifying HIV-1 status of a sample from a subject as recent or long standing, the method comprising:
a. Measuring in the sample the avidity of IgG3 antibodies to p66 (measurement p66X1) (e.g., p66 from (Protein Sciences, catalog number 2008)),
b. Measuring in the sample the avidity of IgM antibodies binding to gp41 (measurement gp41X2), gp41 (for example, gp41 comprises the full-length protein),
c. Measuring in the sample the avidity of IgG4 antibodies binding to envelope WITO4160 gp140 (measurement WITOX3) comprising LANL database accessioning number AY835451,
d. Measuring in the sample binding of IgM to gp41 (measurement gp41X4) (for example, gp41 comprises the full-length protein),
e. Using measurements X1, X2, X3 and X4 to determine recent (R) or longstanding (L) discriminant value (DVⱼ is used interchangeably Cⱼ), wherein “j” is recent (R) or longstanding (L) class, wherein cut off period for R is nine months, and wherein
   i. DV_{R}=co_{R}+c_{1R}X1+c_{2R}X2+c_{3R}X3+c_{4R}X4, wherein in some embodiments R classification coefficients are as follows: Table 41, and
   ii. DV_{L}=co_{L}+c_{1L}X1+c_{2L}X2+c_{3L}X3+c_{4L}X4, wherein in some embodiments L classification coefficients are as follows: Table 41; and wherein if DV_{R} is larger than DV_{L}, the sample is classified as Recent, or wherein if DV_{L} is larger than DV_{R} the sample is classified as Longstanding.

In some embodiments, gp41 comprises Clade B (MN) gp41 protein (for example gp41 protein from ImmunoDx, catalog number 1091).

In some aspects, the invention provides methods for classifying HIV-1 status of a sample from a subject as recent or long standing, the method comprising: detecting and quantifying the formation of antibody-antigen complexes, wherein the antibodies-antigen complexes are avidity of IgG3 antibodies to p66 (measurement p66X1), avidity of IgM antibodies binding to gp41 (measurement gp41X2), avidity of IgG4 antibodies binding to envelope WITO4160 gp140 comprising LANL database accessioning number AY835451 (measurement WITOX3), binding of IgM to gp41 immunodomninant domain (IG) (measurement gp41X4), and using measurements X1, X2, X3 and X4 to determine recent (R) or longstanding (L) discriminant value (DVⱼ is used interchangeably Cⱼ), wherein “j” is recent (R) or longstanding (L) class, wherein cut off period for R is nine months, and wherein
i. DV_{R}=co_{R}+c_{1R}X1+c_{2R}X2+c_{3R}X3+c_{4R}X4, wherein in some embodiments R classification coefficients are as follows: Table 41, and
ii. DV_{L}=co_{L}+c_{1L}X1+c_{2L}X2+c_{3L}X3+c_{4L}X4, wherein in some embodiments L classification coefficients are as follows: Table 41; and wherein if DV_{R} is larger than DV_{L}, the sample is classified as Recent, or wherein if DV_{L} is larger than DV_{R} the sample is classified as Longstanding.

In some aspects the invention provides a kit comprising a selection of HIV-1 antigens for detecting and quantifying the formation of antibody-antigen complexes from a biological sample, wherein the antibodies-antigen complexes are binding of IgG3 antibodies to Clade C gp140 envelope (measurement X1), avidity of IgG4 antibodies binding to T/F Clade C gp140 envelope (measurement X2), avidity of IgG4 antibodies binding to Clade B gp140 envelope (measurement X3), avidity of IgG to gp41 immunodomninant domain (IG) (measurement X4), wherein the avidity of IgG gp41 ID is determined as difference between IgG Citrate gp41 ID epitope and IgG PBS gp41 ID epitope, using measurements X1, X2, X3 and X4 to determine recent (R) or longstanding (L) discriminant value (DVⱼ is used interchangeably Cⱼ), wherein “j” is recent (R) or longstanding (L) class, wherein cut off period for R is nine months, and wherein
i. DV_{R}=co_{R}+c_{1R}X1+c_{2R}X2+c_{3R}X3+c_{4R}X4, wherein in some embodiments R classification coefficients are as follows: Table 40 or Table 41, and
ii. DV_{L}=co_{L}+c_{1L}X1+c_{2L}X2+c_{3L}X3+c_{4L}X4, wherein in some embodiments L classification coefficients are as follows: Table 40 or Table 41; and
wherein if DV_{R} is larger than DV_{L}, the sample is classified as Recent, or
wherein if DV_{L} is larger than DV_{R} the sample is classified as Longstanding.

In some embodiments of the kits and methods, the HIV-1 antigens are adapted to be used in a quantitative or semi-quantitative assay to yield measurements. In some embodiments of the kits and methods, the HIV-1 antigens are immobilized on a solid support. In some embodiments the HIV-1 antigens are conjugated to beads. In some embodiments, the antigens are adapted for use in BAMA assay. In some embodiments, the antigens are adapted for use in lateral flow assay.

In some embodiments, the kits further comprise instructions on how to carry out the steps of any one of the methods of the invention.

In some embodiments, the instructions include instructions on determining DV_{R} and DV_{L}, and the classification/discriminant coefficients of Table 40 or Table 41.

The invention contemplates non-limiting embodiments of kits and methods, wherein the sample is plasma, serum, finger stick blood, dried blood spot (DBS), whole blood, saliva, urine, mucosal fluid, or any other suitable biological sample.

In some embodiments, the invention provides a set of antigen/antibody biomarkers that could be used in a DFA analysis to classify HIV-1 samples comprising HIV-1 antibodies as recent or longstanding infections.

In some aspects the invention provides a method to determine HIV-1 incidence in a subject comprising: determining in a biological test sample isolated from a subject the concentration of IgG antibodies to an HIV-1 antigen, the concentration of IgA antibodies to an HIV-1 antigen, or the combination thereof, comparing the test sample concentration to concentrations determined in a reference sample from a recently or a chronically infected individual, wherein a test sample concentration of IgA, IgG, or the combination thereof within the concentration range of a recent sample, is indicative of a recent infection.

In some embodiments of the methods determining includes measuring the concentration of IgG antibodies to an HIV-1 antigen and the concentration of IgA antibodies to an HIV-1 antigen. In certain embodiments, determining includes contacting the HIV-1 antigens with a biological test sample form the subject under condition suitable to detect immune complexes so as to determine the concentrations and avidity of the IgG and IgA antibodies to the HIV-1 antigens and/or epitopes. In certain embodiments the HIV-1 antigens are multiplexed on a single platform. In certain embodiments the multiplexed platform includes all necessary controls. In certain embodiments the biological test sample includes but not limited to plasma, serum, saliva, urine, or any combination thereof.

In some embodiments, determining includes measurement of the presence, magnitude and/or avidity of antibody-antigen binding by any suitable assay.

In some embodiments, the IgG isotype is IgG1, IgG2, IgG3, Ig4 or any combination thereof. In some embodiments, the IgA isotype is IgA1, IgA2 or any combination thereof. In some embodiments, the IgA form is mIgA, dIgA, SIgA, or any combination thereof.

In some embodiments, the HIV-1 antigen for determining IgA or IgG concentration/response is Envelope (gp120, gp140, including consensus gp120 envelopes, gp41), Gag, Integrase, RT, Nef, Tat, Rev proteins and/or epitopes and multiclade panels of gp120, and epitope specific responses, or any combination thereof. In some embodiments, the HIV-1 antigen for determining IgG concentration/response is gp41 immunodominant and KE region, V3, V2, C1 and C5 epitopes.

In some embodiments, the HIV-1 antigen for determining IgA concentration/response is immunodominant and KE regions of gp41, V3, V2, C1 and C5 epitopes or any combination thereof.

In some embodiments, the HIV-1 antigen for determining IgA or IgG concentration/response is a specific epitope, peptide or the combination thereof described herein.

In some embodiments, the method can further comprise determining the avidity of an antibody-HIV-1 antigen complex, wherein a test sample avidity measurements within the avidity range of a recent sample, is indicative of a recent infection. In certain embodiments the antibody is IgG. In certain embodiments the antibody is IgM. In other embodiments the antibody is IgA. In certain embodiments, the HIV-1 antigen for determining avidity of an antibody-HIV-1 complex is Envelope (gp120, including consensus gp120 envelopes, gp41), Gag, Integrase, RT, Nef, Tat, Rev proteins and/or epitopes and multiclade panels of gp120, and epitope specific responses, or any combination thereof. In certain embodiments the epitopes are immunodominant and KE regions of gp41, V3, V2, C1 and C5 epitopes and identified epitopes in Gag, Rev, Pro, Integrase, Rev as described herein. In certain embodiments, the measured responses and antigens are shown in Example 3. comprising determining the ratio of antibody forms and ratio of antigen. In certain embodiments, the determine ratio is the ratio of IgA forms in a sample.

In some aspects, the invention provides a kit comprising a selection of HIV-1 antigens to determine the concentrations and/or avidity of IgG, IgM, and IgA antibodies to the HIV-1 antigens in a biological sample. In some embodiments, the HIV-1 antigens are deposited on a solid support. In some embodiments the antigens are conjugated to beads.

In some embodiments, the kit further provides coefficients and/or reference values for recently or a chronically infected reference sample(s) and instructions for test sample data analysis of any one of the determined measurements of magnitude, avidity, antibody ratio, or the combination thereof so as to determine whether the test sample is within the range measured in a reference sample from a recently or a chronically infected individual.

In certain aspects the invention provides methods to determine HIV-1 incidence in a subject. In certain aspects, the invention provides a method to determine HIV-1 incidence in a subject the method comprising determining in a sample from the subject types, maturation and kinetics of HIV-1 antibody isotypes and subclasses responses. In certain aspects, the invention provides a method to determine HIV-1 incidence in a subject the method comprising determining in a sample from the subject concentration and/or avidity of HIV-1 antibody isotypes and subclasses responses to a combination of antigens. In certain embodiments, the measurements of the test sample are compared to a reference sample from a recently or chronically infected individuals. In certain embodiments, the test sample measurements or combination of various measurements is compared to a calculated reference value, or a range of reference values, which is calculated so as to distinguish a recent from chronic infection. In certain embodiments, the combination of measurements is described in Example 3. In certain embodiments, the combination of measurements is described in Example 3.

In certain aspects the invention provides a method to determine HIV-1 incidence in a subject comprising: determining in a biological test sample isolated from a subject the concentration of IgG antibodies to an HIV-1 antigen, the concentration of IgA antibodies to an HIV-1 antigen, or the combination thereof, and comparing the test sample concentration to concentrations determined in a reference sample from a recently or a chronically infected individual, wherein a test sample concentration of IgA, IgG, or the combination thereof within the concentration range of a recent sample, and outside of the range of a chronic sample is indicative of a recent infection.

In certain embodiments, the methods further comprise measuring the IgM avidity of a biological test sample, measuring the IgG3 avidity of a biological test sample, measuring the IgG4 avidity of a biological test sample, or the combination thereof.

In certain embodiments determining includes measuring the concentration of IgG antibodies to an HIV-1 antigen and the concentration of IgA antibodies to an HIV-1 antigen.

In certain embodiments of the methods of the invention, determining includes contacting the HIV-1 antigens with a biological test sample form the subject under condition suitable to detect immune complexes so as to determine the concentrations and avidity of the IgG and IgA antibodies to the HIV-1 antigens and/or epitopes. In certain embodiments the HIV-1 antigens are multiplexed on a single platform. In certain embodiments the multiplexed platform includes all necessary controls. In certain embodiments the biological test sample includes but not limited to plasma, serum, saliva, urine, or any combination thereof.

In certain embodiments determining includes measurement of the magnitude or avidity of antibody-antigen binding by any suitable assay.

In certain embodiments the IgG isotype is IgG1, IgG2, IgG3, Ig4 or any combination thereof.

In certain embodiments the IgA isotype is IgA1, IgA2 or any combination thereof. In certain embodiments the IgA form is mIgA, dIgA, SIgA, or any combination thereof.

In certain embodiments the IgG isotype is IgG1, IgG3, or IgG4, and IgA form is _dIgA and mIgA.

In certain embodiments the HIV-1 antigen for determining IgA or IgG concentration/response is Envelope (gp120, including consensus gp120 envelopes, gp41), Gag, Integrase, RT, Nef, Tat, Rev proteins and/or epitopes and multiclade panels of gp120, and epitope specific responses, or any combination thereof. In certain embodiments the HIV-1 antigen for determining IgG concentration/response is gp41 immunodominant and KE region, V3, V2, C1 and C5 epitopes, or any combination thereof. In certain embodiments the HIV-1 antigen for determining IgA concentration/response is immunodominant and KE regions of gp41, V3, V2, C1 and C5 epitopes, or any combination thereof.

In certain embodiments the invention provides methods to measure HIV-1 antibody responses in a subject using the following non-limiting measurements or combinations thereof: (1) three different antibody types, (2) both binding and avidity measurements of antibody responses, and (3) HIV-1 gp41, gp140, and non-Env HIV-1 proteins. In certain embodiments these could include: IgG4 A1 Con gp140 (Subtype A) binding; IgA 1086 gp140 (Subtype C) avidity; IgG4 1086 gp140 (Subtype C) binding; IgG gp41 (subtype B) avidity; IgG p31 binding; IgG3 p66 avidity; IgM gp41 avidity; IgG4 WITO gp140 (Subtype B); IgM gp41 binding, or any combination thereof.

In certain embodiments the HIV-1 antigen for determining IgA or IgG concentration/response is a specific epitope, peptide or the combination thereof described in herein.

In certain embodiments the methods further comprise determining the avidity of an antibody-HIV-1 antigen complex, wherein a test sample avidity measurements within the avidity range of a recent sample, and outside of the range of a chronic sample is indicative of a recent infection. In certain embodiments the antibody is IgG. In other embodiments the antibody is IgA. In certain embodiments the antibody is IgM. In certain embodiments, the HIV-1 antigen for determining avidity of an antibody-HIV-1 complex is Envelope (gp120, including consensus gp120 envelopes, gp41), Gag, Integrase, RT, Nef, Tat, Rev proteins and/or epitopes and multiclade panels of gp120, and epitope specific responses, or any combination thereof. In certain embodiments the epitopes are immunodominant and KE regions of gp41, V3, V2, C1 and C5 epitopes, identified epitopes in Gag, Rev, Pro, Integrase, Rev as described herein, or any combinations thereof.

In certain embodiments the methods further comprise determining the ratio of antibody forms and ratio of antigen, for example but not limited the ratio of IgA forms in a sample.

In certain aspects the invention provides a kit comprising a selection of HIV-1 antigens to determine the concentrations and/or avidity of IgG, IgA antibodies to the HIV-1 antigens in a biological sample. In certain embodiments the HIV-1 antigens are deposited on a solid support. In some embodiments the antigens are conjugated to beads. In certain embodiments the kit further comprises instructions on how to carry out the steps of any one of the above claims. In certain embodiments the antigens are any of the antigens described herein.

In certain embodiments, the instructions in the kit further provide reference values for making a determination, for example but not limited to concentration ranges, threshold values, for recently or a chronically infected reference sample(s). In certain embodiments, the instructions in the kit further provide instructions for test sample data analysis of any one of the determined measurements of magnitude, avidity, antibody ratio, or the combination thereof so as to determine whether the test sample is within the range measured in a reference sample from a recently or a chronically infected individual.

In certain aspects the invention provides a selection of HIV-1 antigens to be used in methods to determine HIV-1 incidence. In certain embodiments the HIV-1 antigens are deposited on a solid support. In certain embodiments, the HIV-1 antigens can be used in a single reaction with a sample to make multiple determinations of antibody concentrations and avidity.

The results from this project will impact current and planned HIV-1 incidence measurements by providing new biological measurements for an HIV-1 incidence algorithm. In addition, the measurements provided as part of this project can be shared with other investigators and used to test other hypotheses or as part of future algorithms.

### BRIEF DESCRIPTION OF THE DRAWINGS

FIG. 1 shows that IgG Ab specificities arise sequentially. Tomaras, et al. (2008) J. Virology, 82:12449; Tomaras et al. Current Opinion in HIV/AIDS 2009; Yates, Tomaras AIDS; Yates, Tomaras Nature Mucosal Immunology 2013. Tomaras G D, Yates N L, Liu P, Qin L, Fouda G G, Chavez L L, Decamp A C, Parks R J, Ashley V C, Lucas J T, Cohen M, Eron J, Hicks C B, Liao H X, Self S G, Landucci G, Forthal D N, Weinhold K J, Keele B F, Hahn B H, Greenberg M L, Morris L, Karim S S, Blattner W A, Montefiori D C, Shaw G M, Perelson A S, Haynes B F. Initial B-cell responses to transmitted human immunodeficiency virus type 1: virion-binding immunoglobulin M (IgM) and IgG antibodies followed by plasma anti-gp41 antibodies with ineffective control of initial viremia. (2008) Journal of Virology, December; 82(24):12449-63. Epub 2008 Oct. 8.PMID: 18842730; Tomaras G D, Haynes B F. HIV-1 Specific Antibody Responses during HIV-1 Infection. (2009) Current Opinion in HIV/AIDS. September; 4(5):373-9. Review. PMID: 20048700; Yates N, Nolan T, Vandergrift N, Stacey, A, Borrow, P, Moody A, Montefiori D, Weinhold K J, Blattner W A, Shattock R, Cohen M, Haynes B F, Tomaras G D. HIV-1 Envelope IgA is Frequently Elicited after Transmission but has an Initial Short Half-Life. (2013) Nature Mucosal Immunology, epub January July; 6(4):692-703.

FIG. 2 shows (FIG. 2A) High Data Content: Binding Antibody Multiplex Assay (BAMA) (Tomaras et al. 2008 J. Virol; Yates, Tomaras 2013 Nature Muc. Immunol.; Eckels et al. 2013 BMC Bioinformatics) and (FIG. 2B) Discovery Tool (Peptide Array) (Tomaras, Shen et al. (2011) Journal of Virology 85:11502; Tomaras G D, Yates N L, Liu P, Qin L, Fouda G G, Chavez L L, Decamp A C, Parks R J, Ashley V C, Lucas J T, Cohen M, Eron J, Hicks C B, Liao H X, Self S G, Landucci G, Forthal D N, Weinhold K J, Keele B F, Hahn B H, Greenberg M L, Morris L, Karim S S, Blattner W A, Montefiori D C, Shaw G M, Perelson A S, Haynes B F. Initial B-cell responses to transmitted human immunodeficiency virus type 1: virion-binding immunoglobulin M (IgM) and IgG antibodies followed by plasma anti-gp41 antibodies with ineffective control of initial viremia. (2008) Journal of Virology, December; 82(24):12449-63. Epub 2008 Oct. 8.PMID: 18842730; Eckels J, Nathe C, Nelson E K, Shoemaker S G, Nostrand E V, Yates N L, Ashley V C, Harris L J, Bollenbeck M, Fong Y, Tomaras G D and Piehler B. Quality control, analysis and secure sharing of Luminex® immunoassay data using the open source LabKey Server platform. BMC Bioinformatics 2013, 14:145; Tomaras G D, Binley J M, Gray E S, Crooks E T, Osawa K, Moore P L, Tumba N, Tong T, Shen X, Yates N L, Decker J, Wibmer C K, Gao F, Alam S M, Easterbrook P, Abdool-Karim S, Kamanga G, Crump J A, Cohen M, Shaw G M, Mascola J R, Haynes B F, Montefiori D C, Morris L. Polyclonal B Cell Responses to Conserved Neutralization Epitopes in a Subset of HIV-1-infected Individuals J. Virol. 2011 November 85 (21): 11502-11519. PMID:21849452.

FIG. 3 is a table showing the presence of Antigen/Antibody Positivity. (Yates, Tomaras (2011) AIDS 25:2089-2097).

FIG. 4 is a table depicting Plasma IgA Biomarkers of HIV-1 Incidence. Changing kinetics of plasma IgA (A1, A2) form (dIgA, SIgA), frequency and avidity to particular epitopes from acute to chronic HIV-1. (Yates, Tomaras et al. (2013) Nature Muc. Immunol. July; 6(4):692-703).

FIG. 5 is a line graph showing Evidence for Different IgA forms During Early acute infection. The frequency, magnitude, and MDR of monomeric and dimeric IgA across multiple antigen specificities (p24 Gag, gp41 epitopes) indicate that a combination of different antigen specific dIgA measurements are candidates for inclusion.

FIG. 6 is a plot showing Antibody/Antigen Measurements Chosen Based on Range of Correlations. One criteria for inclusion of Ab-antigen measurements is low to moderate correlations in order to cover unique immunological space (e.g. N=6 Antibody/Antigen Measurements.

FIG. 7 is a plot depicting the Classification of CEPHIA Panel. Example of a Canonical discriminant function analysis of a selected set of N=6 Antibody/Antigen Measurements (Magnitude and Avidity). The legend—Black: Longstanding Infection (>12 months); Green: Recent Infection (<6 months); Red: Virus Controller (<50 VL); and Blue: HESN/Acute Mix Group Includes: 1 HIV+, 22 HESN, 2 HESN/Acutes.

FIG. 8 is a graph showing Simulation Study of Error Rate Coverage by Number of Antigens Selected (K). Panels of “K” measurements chosen for analysis: Based on “best of class” and/or Wald chi square cutoff of “9”. Parametric discriminant function analysis showing selections from K=3-18. The selection of measurements were independently verified from the discriminant function analysis by bootstrapping random sets of measurements of (3-18) from within the 281 measurements.

FIG. 9 is a schematic of CEPHIA Stage gates.

FIG. 10 is a graph showing Dynamic Ab Specificities and Types: from Acute to Chronic.

FIG. 11 is a schematic showing High Data Content of an HIV-1 Binding Antibody Multiplex Assay (BAMA) and BAMA-Avidity Index.

FIG. 12 are graphs showing CEPHIA/CHAVI of the Best 5 Antigen Set with Misclassification <3%. The solution involved 3 different antibody types (subclasses/isotypes), direct binding and avidity measurements, and Gp41, gp140 and non-Env.

FIG. 13 are graphs of a measurement set of the Pitt Panel 2.

FIG. 14 are graphs showing Pitt Panel 2 best 4 measurements together (canonical 1 value).

FIG. 15 shows graphs of Phenotype of CD4 T cell subsets down-selected for incidence assay.

FIG. 16 is a schematic of cell-associated HIV-1 DNA. 3 commonly detected forms: Total DNA; Integrated; and LTR circles. HIV gag sequence present in all forms. 2-LTR circles: Byproduct of integration; Stable; and Nuclear.

FIG. 17 shows graphs of the NHP model: CD4⁺ T cell subsets with CAVL ratios that change over time. Longitudinal PBMC from over 50 rhesus macaque were sorted into the different CD4 T-cell populations and the ratio of the CAVL within each population was calculated. Shown here are regression analyses of the ratios versus time after infection. We saw the strongest signal in naïve versus either central or transitional memory cell subsets. These populations were thus downselected for analysis in humans.

FIG. 18 shows graphs of CAVL (total HIV-1 DNA) in human CD4⁺ T cell populations: CEPHIA panel. The CEPHIA development panel shows CAVL detected in “recent” and “longstanding,” but not HESN. Additional data required to classify by T-cell subset CAVL ratio.

FIG. 19 shows graphs of 2-LTR circle DNA. 2-LTR DNA undetected in most samples. It is rarely detected in naïve cells of recent (9%), versus 23% of longstandings.

FIG. 20 shows graphs of 2-LTR circle DNA. A subset of “recents” can be separated from “longstandings” by 2-LTR in memory subsets. Combining 2-LTR analysis with gag may completely resolve groups.

FIG. 21 are plots of samples that were tested for binding antibody responses to a panel of 281 antigen/antibody pairs. Results were then analyzed using discriminant function analysis to select sets of 2-7 antigen/antibody pairs that achieved the lowest FRR. The combination of the 4 analytes shown in this figure represent one solution that achieved a 0% FRR (3.5% total error).

FIG. 22 are plots of samples that were tested for binding antibody responses to a panel of 281 antigen/antibody pairs and analyzed using discriminant function analysis. A set of 4 antigen/antibody pairs that achieved a 0% FRR was chosen for additional analysis. The left panel indicates the canonical value obtained using the best set of 4 antigens for samples classified as recent or longstanding. The right panel indicates the Predicted Duration of Infection for samples classified as Recent (<9 months) or Longstanding based upon the canonical value obtained using the best set of 4 antigen/antibody pairs.

FIG. 23 are plots of samples that were tested for binding antibody responses to a panel of 505 antigen/antibody pairs. Results were then analyzed using discriminant function analysis to select sets of 4-5 antigen/antibody pairs that achieved the lowest FRR. The combination of the 4 analytes shown in this figure represent one solution that achieved a 4.5% FRR (6.6% total error).

FIG. 24 are plots of samples that were tested for binding antibody responses to a panel of 505 antigen/antibody pairs and analyzed using discriminant function analysis. A set of 4 antigen/antibody pairs that achieved a 0% FRR was chosen for additional analysis. The left panel indicates the canonical value obtained using the best set of 4 antigens for samples classified as recent or longstanding. The right panel indicates the Predicted Duration of Infection for samples classified as Recent (<9 months) or Longstanding based upon the canonical value obtained using the best set of 4 antigen/antibody pairs.

FIG. 25 shows plots of CEPHIA-CHAVI DP Calculated DV and PDI. DV: β₁=0.12 (p<0.0001), R²=0.77.

FIG. 26 shows plots of the Best 5 Measurement Set, Misclassification <3%.

FIG. 27 shows plots of all subjects (N=4) measurements with misclassification ˜9%. DV: β₁=0.05 (p=0.21), R²=0.02.

FIG. 28 shows plots of all subjects (N=4) measurements with misclassification ˜9%.

FIG. 29 shows plots of no ART subjects (N=4) measurements with misclassification ˜9%. DV: β₁=0.09 (p<0.0001), R²=0.57.

FIG. 30 shows plots of no ART subjects (N=4) measurements with misclassification ˜9%.

FIG. 31 shows plots of the Best 4 Measurement Set from the Pitt Panel 2 New Measurements data set.

FIG. 32 shows plots for Pitt Panel 2 Calculated DV and PDI. DV: β₁=−0.06 (p=0.02), R²=0.12.

FIG. 33 shows multiplex incidence assay strategy.

FIG. 34 shows Env Binding and avidity to multiple IgG subclasses classifies recent vs. longstanding infection. An array of 282 antigen-antibody combinations were analyzed using discriminant function analysis. A. The top 20 solutions with an FRR of X, B. Canonical 1 Score for one of the top solutions includes p66 IgG3 avidity, subtype B T/F WITO gp140 IgG4 avidity, gp41 IgM binding, and gp41 IgM avidity.

FIG. 35 shows Env Binding and avidity to multiple IgG subclasses classifies recent vs. longstanding infection.

FIG. 36 shows Env binding and avidity to multiple IgG subclasses accurately classifies recent vs. longstanding infection. A panel of 505 antigen-antibody combinations was analyzed using discriminant function analysis as described in the methods. The top 4 solutions are presented in panels A-D.

FIG. 37 shows canonical Value determination accurately predicts estimated duration of infection. Panel A—a canonical value was calculated for each sample based upon responses to the 4 analytes described in FIG. 3. Panel B—Predicted duration of infection was calculated based upon the canonical value obtained for each sample. A canonical value of <0 indicates recent infection and a canonical value >0 indicates longstanding infection.

FIG. 38 shows MDRI estimate and confidence intervals for novel BAMA biomarker panel. MDRI was calculated using a method as described herein. Results are presented as the probability of testing recent over time, with 95% confidence intervals presented as dashed blue vertical lines. The nominal cutoff for FRR (T=2 years) is indicated with a red vertical line.

FIG. 39 shows BAMA accuracy and linearity.

FIG. 40 shows limit of detection/quantitation.

FIG. 41 shows BAMA repeatability.

FIG. 42 shows Levey Jennings Antigen Reagent Tracking.

FIG. 43 shows BAMA avidity index.

FIG. 44 shows HIV-1 antibody targets. Pictures adapted (Burton et al., Moore, Binley et al., deCamp, Korber, Montefiori et al.; Pollara, Pazgier, Ferrari et al.; David S. Goodsell, RCSB PDB Nat Med, Bakema et al.)

FIG. 45 shows definition of overall HIV-1 immune response kinetics. McMichael, Borrow, Tomaras et al. Nature Immunology Reviews (2010)

FIG. 46 shows presence antigen/antibody positivity. Yates, Tomaras (2011) AIDS 25:2089-2097.

FIG. 47 shows dynamic antibody response acute to chronic.

FIG. 48 shows linear antibody specificities peptide microarray. HIV-1 peptide microarray: multiple epitopes simultaneously in a small volume of blood plasma/serum, or mucosal fluid for human. HIV-1 Sequences: Env, (Gag, RT) Peptide Microarrays: >2000 overlapping Env peptides (15mer overlapping by 12) covering Groups A, B, C, D, M, CRF01 AE, CRF02AG and 6 vaccine strains C.1086, C.TV-1, C.ZM651, B.MN, A244, 92TH023 (designed by Bette Korber based on Global Diversity). Tomaras, Shen et al. (2011) Journal of Virology 85:11502; Gottardo, Montefiori et al. PloS One 8:e75665; Shen, Tomaras et al. 2015, J. Virology.

FIG. 49 shows high data content: HIV-1 binding antibody multiplex array (BAMA). ˜100 Antigens in a Single Sample; Validated for Clinical Trials; Small volume allows interrogation of multiple sample types (serum, plasma saliva, urine); Assay measures antibody isotype, subclass, and avidity to multiple epitopes simultaneously. Tomaras et al. (2008) J. Virol; Haynes et al. (2012) NEJM; Yates, Tomaras (2013) Nature Mucosal Immunology; Eckels et al. 2013 BMC Bioinformatics; Yates, Tomaras (2014) Science Trans. Medicine.

FIG. 50 shows avidity index assay. 2 well format; developed in collaboration with Bharat Parekh; 15 minute treatment with Na-Citrate, pH 3.0; AI=MFI (CIT)/MFI (PBS)*100.

FIG. 51 shows overview of CEPHIA Assay Development Process. FIND Proposed Stage gates: 1. Candidate biomarker(s) associated with recent HIV infection, able to be reliably measured, potentially useful as classifier (“tuneable” threshold); 2. Classification threshold(s) with MDRI 4-24 months, FRR <15% and consistency of FRR between major subtypes; 3. MDRI and FRR estimates meet minimal criteria of TPP for at least 1 use case. *specimens blinded to developer.

FIG. 52 shows downselected measurements.

FIG. 53 shows classification and predicted duration of infection.

FIG. 54 shows Duke result versus time since EDSC.

FIG. 55 shows validation panel results: MDRI and FRR (untreated patients).

FIG. 56 shows specimens from untreated patients. Assay/Model: Assay used to classify specimens as “recent” or “non-recent;” Subtype: Subtype(s) represented in the panel; VL threshold: Value used to reclassify speciments as “non-recent” if VL lower than or equal to this threshold; MDRI: Mean duration of recent infection in days since EDDI; MDRI LB: lower bound of 95% confidence interval of MDRI; MDRI_UB: upper bound of 95% confidence interval of MDRI; MDRI_n_specimens: Number of specimens with valid measurements used for MDRI calculation; may include multiple specimens from the same subject; MDRI_n_subjects: Number of subjects with valid measurements used for MDRI calculation; MDRI_n_recents: Number of specimens with valid measurements used for MDRI calculation that are classified as “recent” by the assay; FRR: False recent rate (% of subjects with 1 or more specimen collected >2 years from LP-DDI that are classified as “recent” by the assay); FRR_LB: lower bound of 95% confidence interval of FRR; FRR_UB: upper bound of 95% confidence interval of FRR; FFF_n_specimens: Number of specimens with valid measurements used for FRR calculation; may include multiple specimens from the same subject; FRR_n_subjects: Number of subjects with valid measurements used for FRR calculation (denominator); FRR_n_recent: Number of subjects with majority of valid measurements classified as “recent” by the assay (numerator); General note: subject-level bootstrapping (MDRI) and subject-level classification (FRR) because a subject's measurements are not statistically independent; General note: EDDI is estimated to occur approximately 24 days prior to Western Blot seroconversion; Prior CEPHIA estimates were relative to WB. EDDI is estimated date of first detectibility using a “1 copy VL assay”; CIs are derived from bootstrap resampling (with replacement) of subjects, not measurements; LP-DDI is the “right side” of the interval during which first detectibility on “1 copy VL assay” is estimated to have occurred, whereas EDDI is the midpoint of this interval; CI is derived from the proportion “recent” treated as a binomial probability; If exactly half of subject's measurements are classified “recent” 0.5, otherwise 1 or 0 according to majority classification.

FIG. 57 shows specimens from ART treated patients. Assay/Model: Assay used to classify specimens as “recent” or “non-recent;” Subtype: Subtype(s) represented in the panel; VL threshold: Value used to reclassify speciments as “non-recent” if VL lower than or equal to this threshold; MDRI: Mean duration of recent infection in days since EDDI; MDRI LB: lower bound of 95% confidence interval of MDRI; MDRI_UB: upper bound of 95% confidence interval of MDRI; MDRI_n_specimens: Number of specimens with valid measurements used for MDRI calculation; may include multiple specimens from the same subject; MDRI_n_subjects: Number of subjects with valid measurements used for MDRI calculation; MDRI_n_recents: Number of specimens with valid measurements used for MDRI calculation that are classified as “recent” by the assay; FRR: False recent rate (% of subjects with 1 or more specimen collected >2 years from LP-DDI that are classified as “recent” by the assay); FRR_LB: lower bound of 95% confidence interval of FRR; FRR_UB: upper bound of 95% confidence interval of FRR; FFF_n_specimens: Number of specimens with valid measurements used for FRR calculation; may include multiple specimens from the same subject; FRR_n_subjects: Number of subjects with valid measurements used for FRR calculation (denominator); FRR_n_recent: Number of subjects with majority of valid measurements classified as “recent” by the assay (numerator); General note: subject-level bootstrapping (MDRI) and subject-level classification (FRR) because a subject's measurements are not statistically independent; General note: EDDI is estimated to occur approximately 24 days prior to Western Blot seroconversion; Prior CEPHIA estimates were relative to WB. EDDI is estimated date of first detectibility using a “1 copy VL assay”; CIs are derived from bootstrap resampling (with replacement) of subjects, not measurements; LP-DDI is the “right side” of the interval during which first detectibility on “1 copy VL assay” is estimated to have occurred, whereas EDDI is the midpoint of this interval; CI is derived from the proportion “recent” treated as a binomial probability; If exactly half of subject's measurements are classified “recent” 0.5, otherwise 1 or 0 according to majority classification.

FIG. 58 shows validation panel results: MDRI and FRR. N (all subtypes): 131 (MDRI), 134 (FRR untreated), 58 (FRR treated); N (nonB): 76 (MDRI), 75 (FRR untreated), 9 (FRR treated—not shown)

FIG. 59 shows Envelope binding and avidity to multiple IgG subclasses accurately classifies recent versus longstanding infection. A panel of 505 antigen-antibody combinations (FIG. 61) was analyzed using discriminant function analysis as described in Ex.9 methods. The top 4 Antibody-Antigen (Ab-Ag) combinations are presented in panel A. Panel B presents the canonical score for the solution with the top 4 Ab-Ag combinations. PBS=Phosphate Buffered Saline; CIT=Na-Citrate, pH 4.0.

FIG. 60 shows multiple sets of 4 HIV-1 specific analytes classify recent infection with 0% False Recent Rate. An array of 282 antigen-antibody combinations was tested via Binding Antibody Multiplex Assay (BAMA) and subsequently analyzed using discriminant function analysis. A. The top measurements resulting in an False Recent Rate (FRR) of 0%, B. Canonical 1 Score for one of the top solutions includes p66 IgG3 avidity, subtype B Transmitted Founder (T/F) WITO gp140 IgG4 avidity, gp41 IgM binding, and gp41 IgM avidity. C. Group=Classification based on days from Estimated Date of Seroconverstion (EDSC) less than or greater than 270 days. Assigned group=classification predicted by canonical values obtained via BAMA.

FIG. 61 shows list of 505 Ab/Ag pairs tested.

FIG. 62 shows a list of a subset of antibody/antigens pairs that could be used in alternative embodiments to determine measurement X1-X4. The criteria for selection of this subset among the 505 Ab/Ag pair was as follows: Ab/antigens that fall in the top solutions for the Pitt Panel 2 (See FIG. 60) and for the downselected panel (from the 505 antigen/antibody combinations). Solutions that included these Ab/Ag pairs had an error rate <10% in the analysis, and thus are good candidates for use in alternative embodiments. Sequences or accessioning numbers from the LANL database are indicated. For B.con03 ID, different embodiments contemplate any and all isotypes, as this is a known biomarker for incidence studies and highly biologically relevant in an incidence algorithm. The invention also contemplates using sequence in both tetramer and non-tetramer form as they perform equivalently. Bio-V3 peptides—the invention contemplates use of sequences for Clades A, B, C and D as any of those would likely score highly depending on the target population, and the sequences are highly similar. The Bio-KKK where seen is a leader sequence for the peptide, and not part of HIV. The buffer for measurement is indicated. For CIT this is important (avidity in the presence of Na-citrate buffer, pH 4.0). For PBS, this indicates all binding of the antigen to the antibody under typical assay conditions (i.e. in diluent and in PBS; the claim should not be restricted to binding just in PBS).

FIG. 63 shows downselected antigen panel.

FIG. 64 shows misclassified specimens.

FIG. 65 shows draft target product profile.

FIG. 66 shows use cases.

FIG. 67 shows specimens from patients on ART and VL<40 at time of specimen collection.

FIG. 68 shows patient specimens from November.

FIG. 69 shows patient specimens from November.

### DETAILED DESCRIPTION OF THE INVENTION

In certain aspects, the invention provides methods to identify novel and accurate markers of recent HIV-1 infection. In certain aspects, the invention provides methods and reagents to determine incidence of HIV-1 infection. Such methods are for use in evaluating HIV/AIDS prevention programs among populations at highest risk.

Monitoring of HIV-1 infection in different subpopulations to more precisely target prevention resources and to determine the effectiveness of prevention programs requires accurate measurement of HIV-1 incidence. Incidence tests are in use; however, they have an unacceptable level of false recents (A2). Thus, new immunological and/or virus biomarkers of recent infection are needed.

The strategy toward the development of a global HIV-1 incidence assay, to define recent from chronic HIV-1 infection, is based on the maturation and kinetics of HIV-1 antibody isotypes and subclasses with recognition of different components of HIV-1 likely resulting from the early depletion of CD4+ T cells and destruction of germinal centers in acute infection.

The first objective is to determine which antibody subclasses and forms of multiple antigen/epitope specificities and measurements (presence/absence, concentration, avidity, ratios) in the same assay test can distinguish recent from chronic infection. The testing phase involves testing three inter-related approaches. The first is to critically evaluate new antibody measurements (antibody types and epitope specificities) including qualitative, quantitative and avidity measurements for each. The second is to test these measurements, alone or as part of a multi-parameter antibody assay, with known misclassified samples (CEPHIA) and acute and recent infection samples (e.g. CHAVI). This is a targeted approach that will allow us to quickly downselect and identify those assays for further evaluation. The third is to evaluate each of these antibody measurements in saliva/oral fluid and urine. In total, we will test the new concepts of temporal concentrations of plasma antibody and use multiplexed antibody tests measured simultaneously in an easy-to-obtain specimen type.

Current HIV-1 Incidence Measurements

Previous work to estimate HIV-1 incidence included prospective cohort studies that followed at-risk populations through serial blood sample testing to determine acquisition of HIV-1 infection. This method of incidence testing proved to be inadequate due to cost, logistics and selection bias of enrolling and following individuals in a study. HIV-1 antibody tests were developed to improve upon these methods (A8, A18). However these current tests remain insufficient for the specifications of incidence testing due the high number of misclassifications that can result, particularly in non-clade B populations (A11-13, A17, A19). Current strategies to measure HIV-1 incidence are based on the BED (antibody responses to a gp41 epitope from subtypes B, E and D by a capture ELISA), avidity test using a modified third generation anti-HIV assay, measurement of the antibody responses to and less-sensitive (“detuned assays”) based on binding antibody responses, and a method entitled “Serologic Testing Algorithm for Recent HIV Seroconversion (STARHS) that employed sensitive and less sensitive commercially available assays (that become removed from the market as they are outdated). However these current tests result in a high number of misclassifications and may overestimate incidence, particularly in non-clade B populations (A12, A17, A19). Furthermore, these incidence tests have not been proven to be accurate when examined cross-sectionally across multiple HIV-1 clades, in the presence of antiretroviral therapy, and in persons with low CD4 counts. Thus, development of new methods are needed to address this global need.

A cross-sectional biomarker based incidence estimate requires (i) a biomarker whose presence (or absence) distinguishes recent from chronic infection (ii) an estimate of the mean “window period” during which time the biomarker is present (or absent). The evolution and differential kinetics of the HIV antigen specific humoral immune response during the early stages of disease form the basis for defining the window period and the development of an incidence test. The initial humoral immune response to HIV-1 possesses certain characteristics that vary widely between individuals as well as responses that are more conserved. Moreover, HIV-1 infects CD4+ T cells during acute infection and cell associated viral load (CAVL) has been demonstrated to predict clinical outcome (A7). The infection of naïve vs effector memory CD4+ T cells may be disproportionate between recent and chronic infection. The key to developing an incidence test to meet the two requirements (i and ii) above will be to define those immune measurements (alone and in combination) that are the least variable among individuals in populations that contain multiple clades, antiretroviral treatment, and low CD4 counts.

Comparative Advantage of the Inventive Methods and Reagents

The comparative advantage of our approach involves the following unique strategies: 1) Multiplex antibody measurements with single sample 2) New antibody isotype (IgA, dIgA), 3) New antigens already proven to distinguish recent from chronic (RT, gp41 IgG3 (in addition to Gag IgG3), 4) Identification of new epitopes in Envelope, RT, Gag, Rev, Integrase, Protease, Nef, Tat for presence/absence of antibody, antibody quantity and avidity, 5) Improved assays sensitivity to better detect low antibody levels 6) New specimen type: Inclusion of assays for Saliva/Urine. The current assays do not include any of these measurements.

Several laboratory based methods of incidence estimation have been developed and in use for the past 10-15 years. Early methods focused primarily on detection of HIV RNA or p24 antigen, which are not ideal for incidence estimation due to the short “window period” present prior to HIV-1 seroconversion. Newer methods have focused on the presence or absence of antibodies and antibody avidity to the HIV-1 envelope region, including the immunodominant region (BED, Vironstika detuned assay, IDE-V3, Vitros, Bio-Rad avidity assays, Architect avidity, LAg avidity, AxSYM avidity). While antibody and avidity assays have improved incidence detection as compared to RNA/p24 antigen assays, the field still faces the challenges of mis-classification and high false recent rates. These misclassifications are mainly due to cross-clade differences, ART treatment, or vaccination. We are providing methods and reagents to define new antibody parameters and avidity index to multiple antigens, in order to decrease mis-classifications due to cross-clade differences, ART treatment, and vaccination. Initial work in our laboratory has compared antibody titer and half-life of multiple HIV-1 antigens (including Env, Gag, and RT) in both acute and chronic infection as well as in Clade B and C subjects on and off ART. Those analyses uncovered antibody parameters that may be used in combination with current algorithms to improve the classification of subjects that would be misclassified with existing incidence methods. In certain aspects the invention provides methods examining additional novel antibody parameters (secretory, monomeric and dimeric IgA), in addition to avidity index to non-envelope proteins.

In certain aspects the invention provides that, the inclusion of titer and avidity to antigens not included in current vaccination strategies (i.e. RT, Integrase) would provide a means of distinguishing true infection from vaccination, which is a critical need moving forward.

Differences in Antibodies Between Recent and Chronic Infection

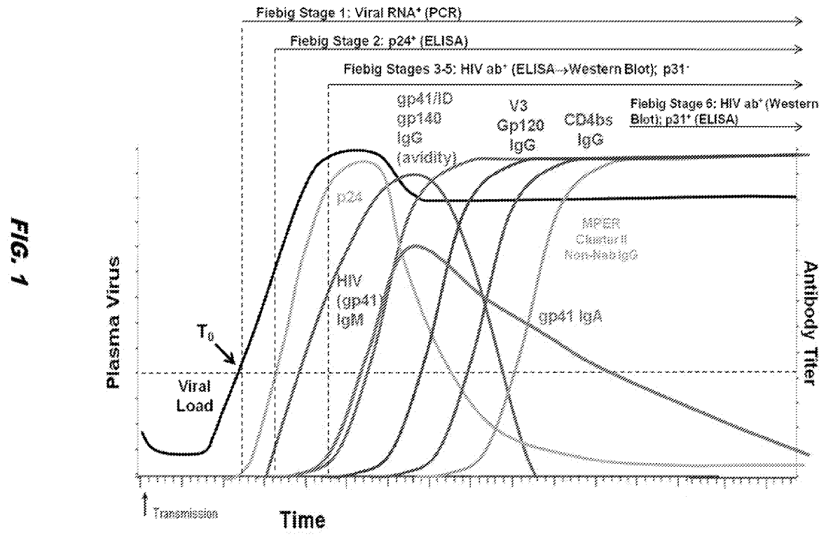

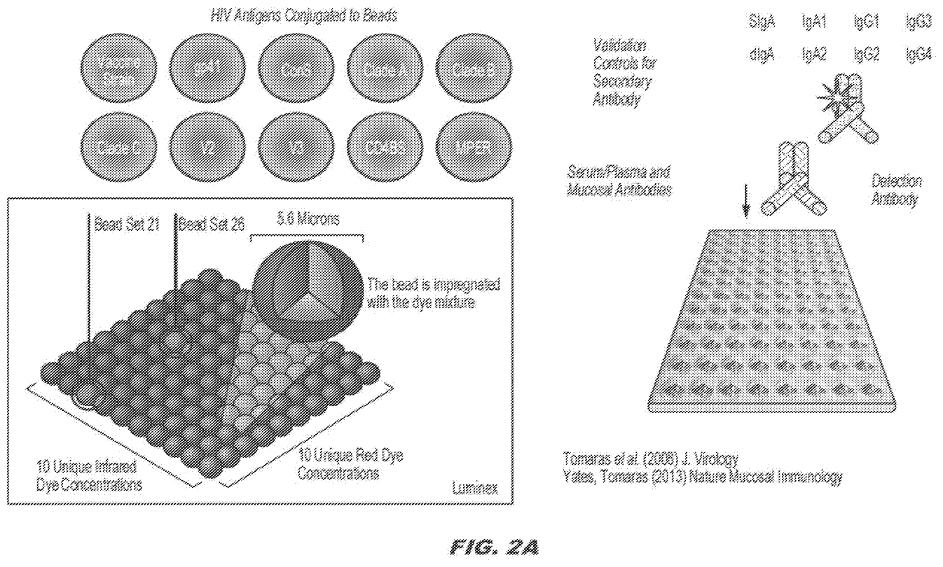

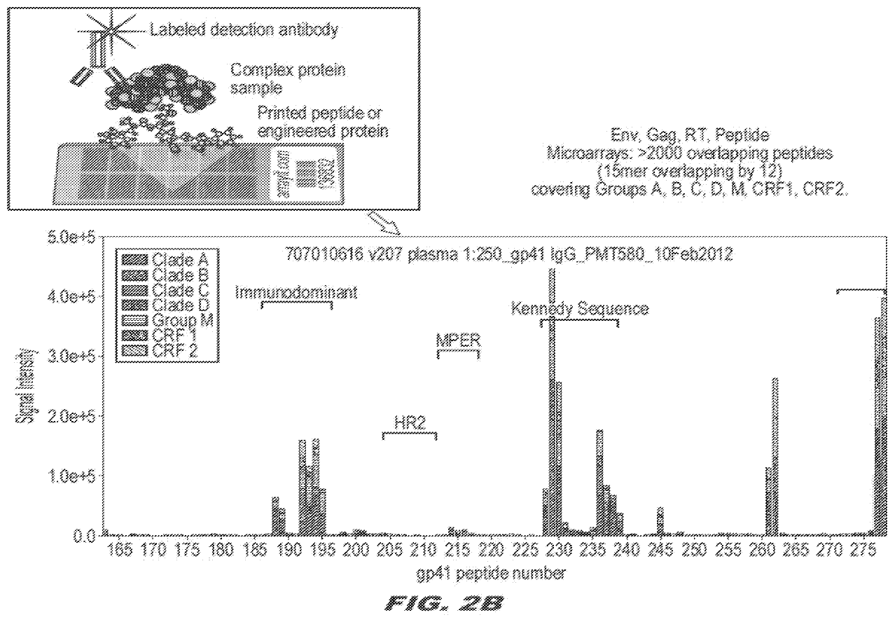

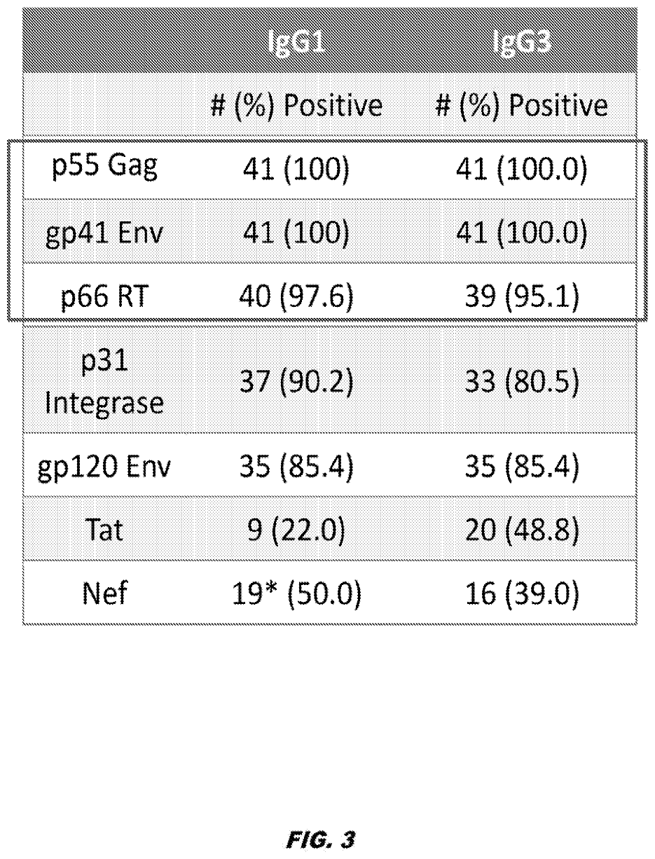

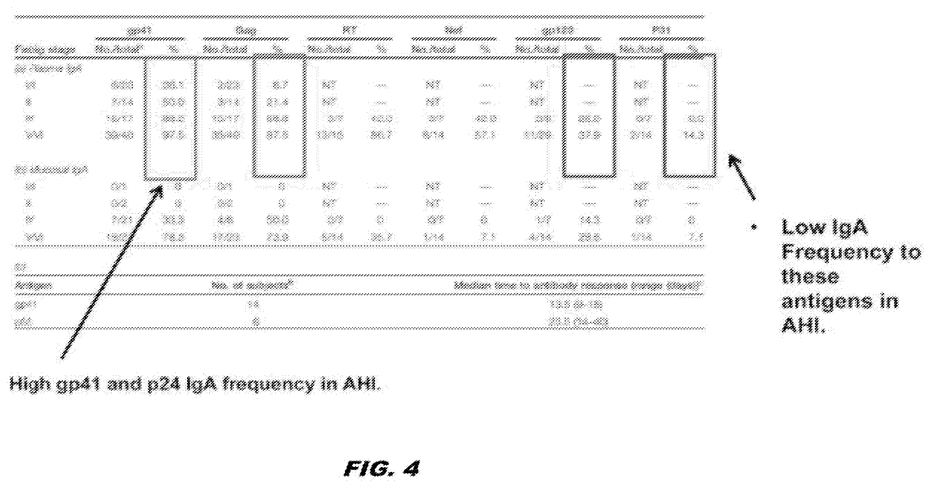

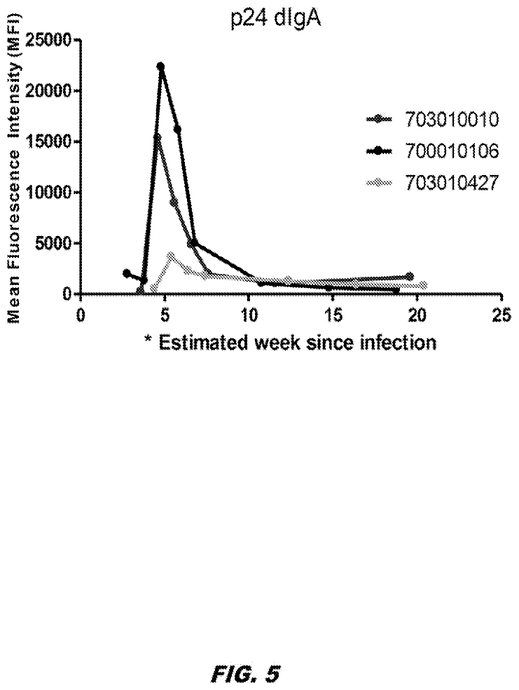

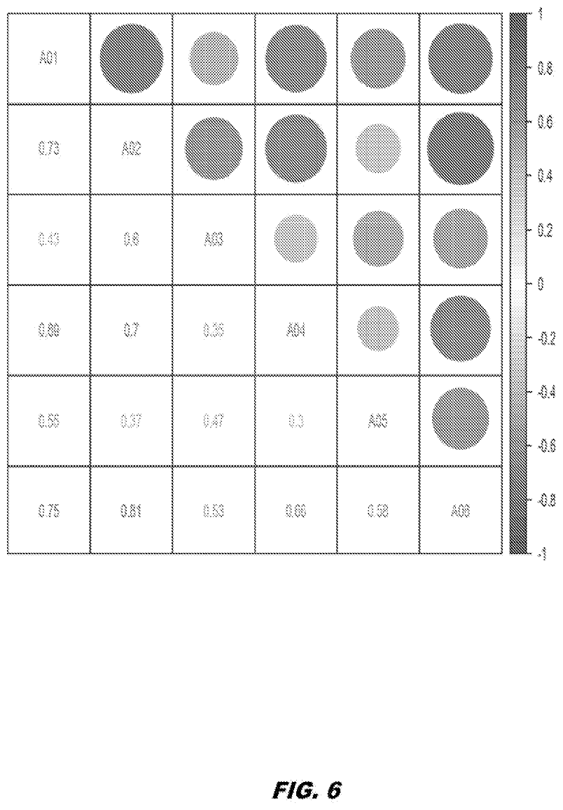

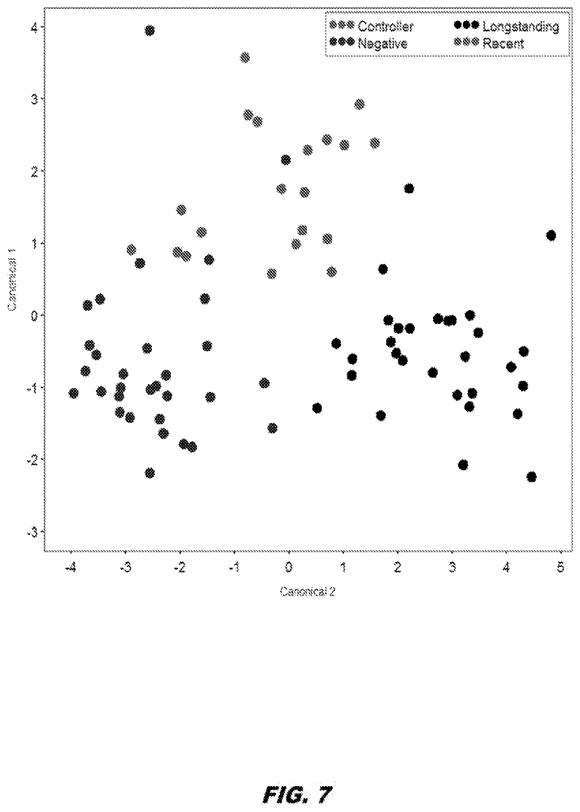

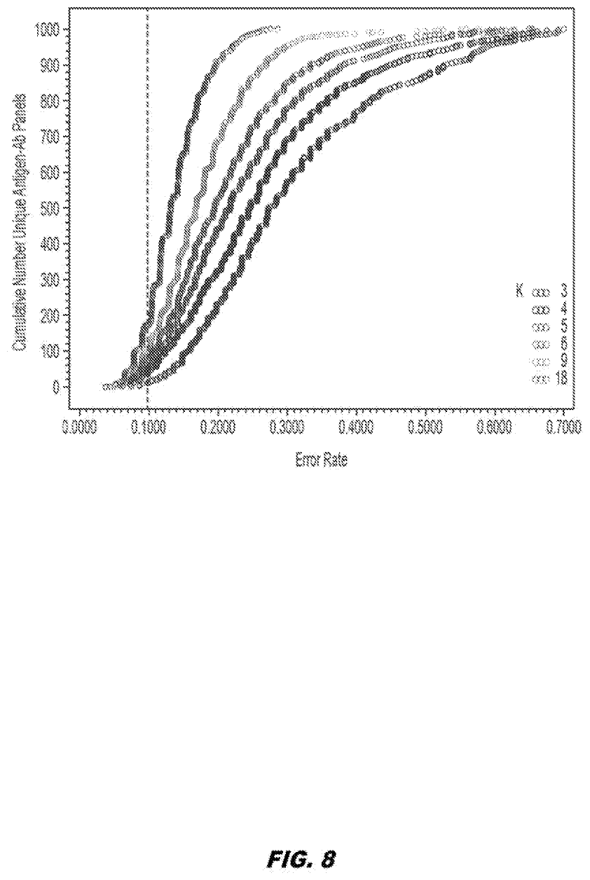

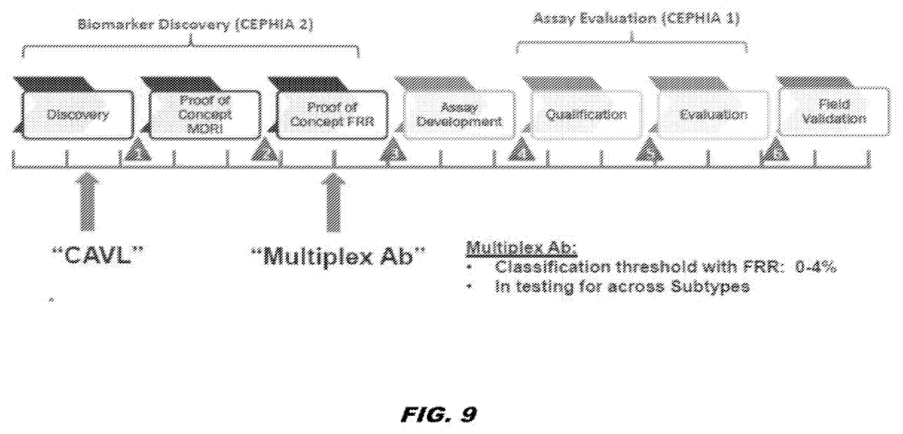

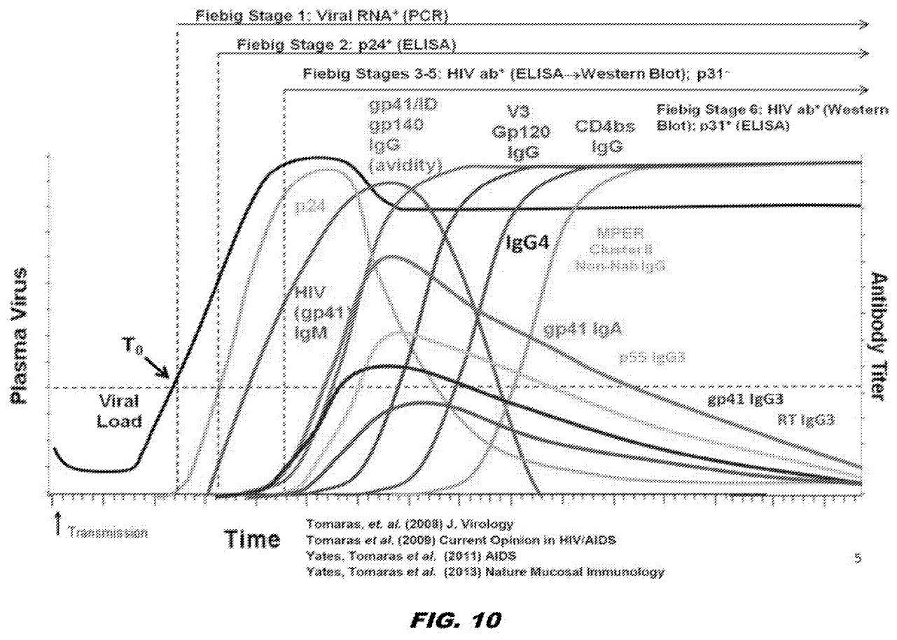

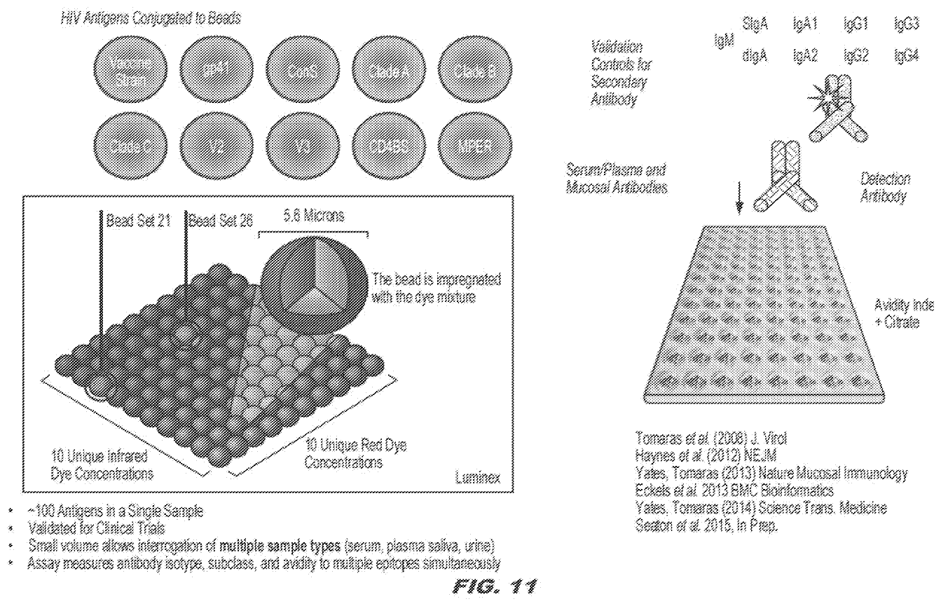

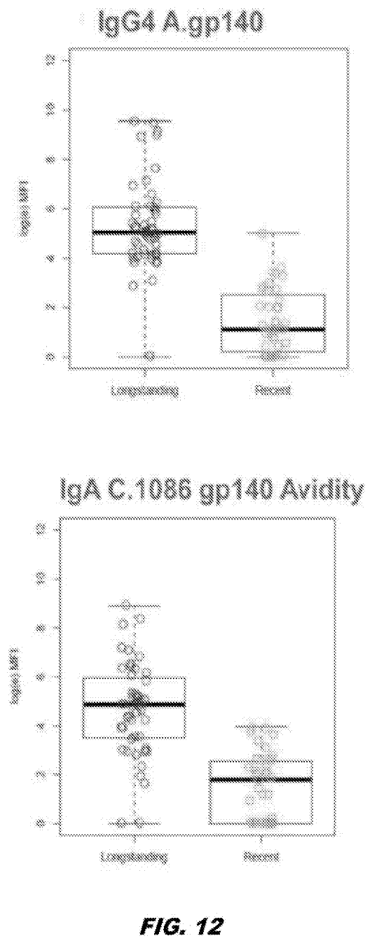

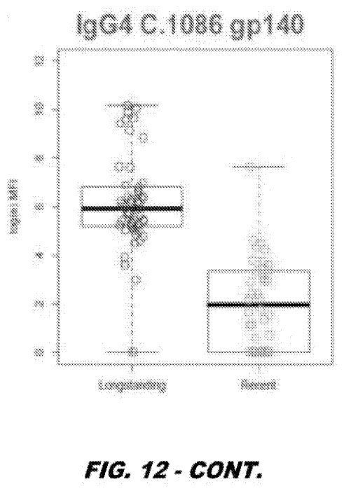

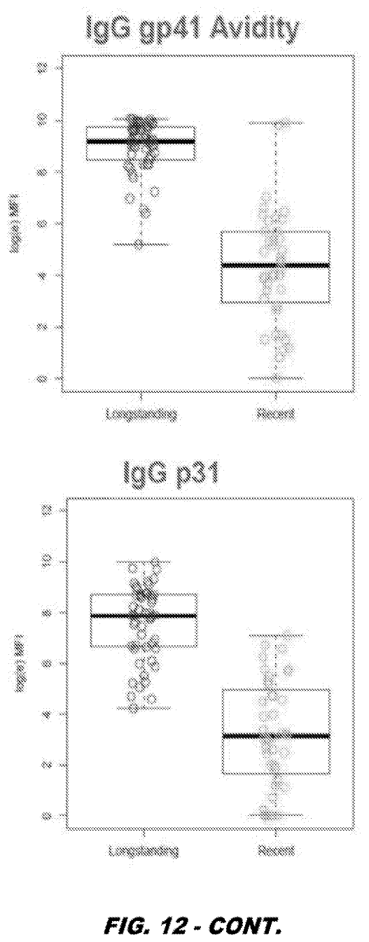

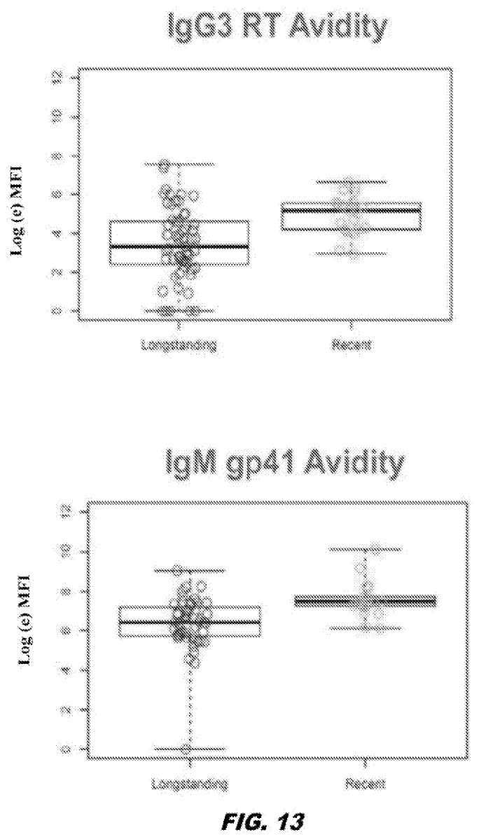

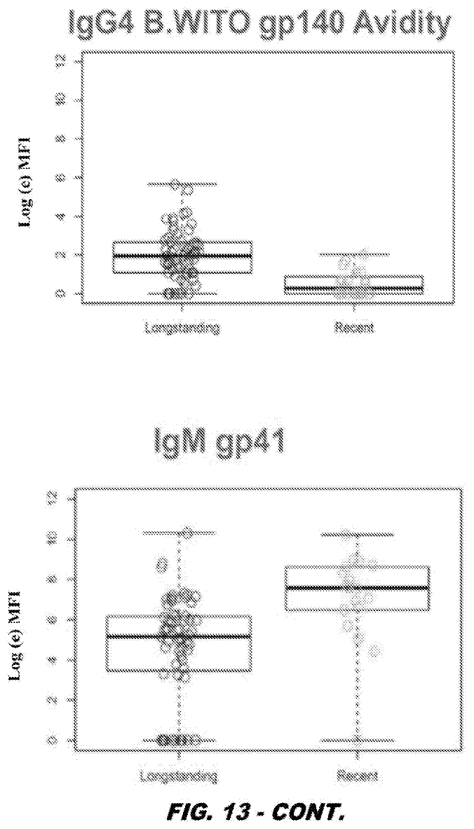

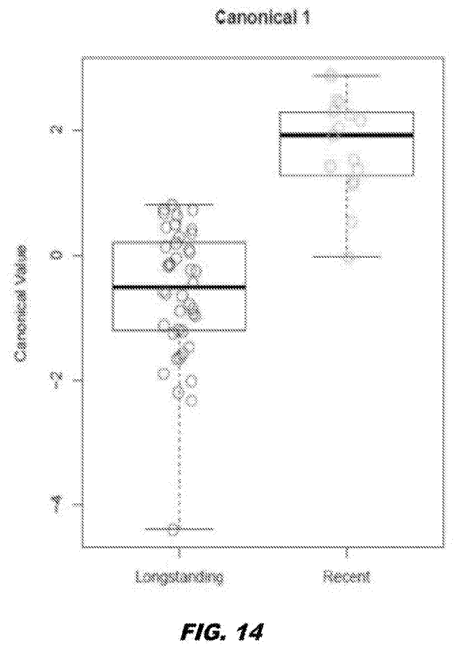

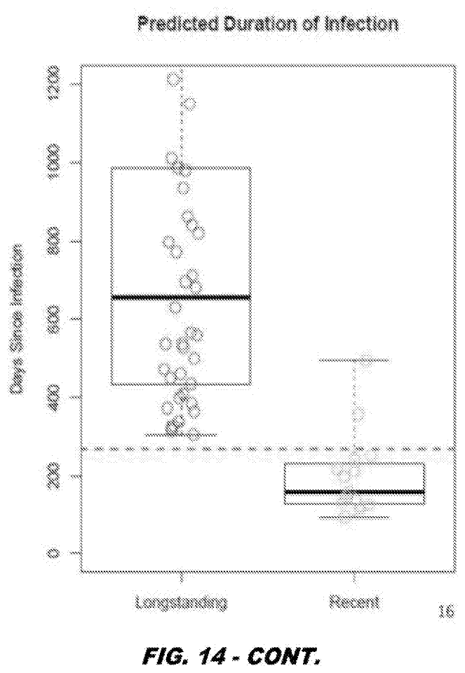

The initial B cell response to HIV-1 is characterized by a staged progression of appearance of non-neutralizing anti-Env antibodies (FIG. 1). Immune complexes appear within days after plasma viremia is detectable and are part of the initial response to HIV-1 transmission. The first free HIV specific antibodies that are produced after transmission are gp41 IgM antibodies (A22). IgG antibodies to the HIV envelope are elicited sequentially with a delay in anti-gp120 antibodies (A22). Epitope specific response to gp41 appear first to the immunodominant region (ID) and in the V3 region for gp120. Antibodies to the CD4BS and non-neutralizing antibodies to the MPER arise later in infection (A1, A22). IgA antibodies to the HIV envelope appear early but in many subjects show an immediate decline (Yates, Tomaras et al, submitted 2012), in contrast to anti-Gag antibodies that begin to rise at this time. Further characterization of IgG isotype by western blot indicated that IgG3 antibodies to Gag decline in acute HIV-1 infection (A24). We have also shown that IgG3 antibody responses to p55 Gag decline in acute infection along with gp41 Env, and p66 RT, in contrast to the same antigen specific IgG1 responses. We determined the antibody concentrations at the half-life and nadir (150 days) that have the least variation among the antibody-antigen measurements (IgG1, IgG3 to Env, Gag, Nef, RT p31 Integrase, and Tat)(25).

HIV-1 Binding Antibody Multiplex Assay (BAMA)

We have developed an HIV-1 custom luminex assay that we have utilized for the measurement of HIV antibody responses in acute and chronic infection (A3, A14, A22) and for the assessment of vaccine elicited responses, the RV144 correlates analyses (9) and in multiple samples types including saliva and urine (CHAVI002 Exposed Uninfected, Shen X, Haynes B, Tomaras G. HIV-1 Specific Antibodies in CHAVI 002. In preparation. 2014). This assay can provide a comprehensive evaluation of the breadth and epitope specificity through the use of gp140 proteins from multiple clades (A, B, C, AE, G, and ancestral, or consensus envelopes), different HIV antigens (p24, p55 Gag, p31 Integrase, p66 RT, Nef and Tat) and peptide specific sequences covering the MPER, immunodominant epitope (ID), C1, V2, V3, and C5 etc. We have determined that HIV Env gp41 and ConS gp140 are very sensitive for detecting antibody responses from multiple clades and are equally or more sensitive than subject derived autologous envelope for the detection of antibody responses (A22).

IgG3 Gag, gp41 and RT Measurements

We have previously used quantitative HIV-1 multiplex antibody assays and exponential decay models to calculate concentrations of IgG1 and IgG3 antibodies to HIV envelope proteins including gp140 consensus oligomers, gp120, and gp41, and to p66 (Reverse Transcriptase), p31 (Integrase), Tat, Nef, and p55 (Gag) proteins during acute/recent HIV-1 infection (A25). We studied patients enrolled during acute HIV-1 infection (AHI), each with between 5 and 20 visits (average of 7.9 visits per subject) and some that went on ART (A5, A15). We found that IgG3 specific responses to gp41 peaked before those to p55 and p66 and despite differential timing of induction of these responses, IgG3 antibody to p55, gp41, and p66 each consistently declined after acute infection, unlike the IgG1 responses to the same antigens.

We observed a decline in p55Gag-specific, gp41Env-specific and p66RT-specific IgG3 responses with a concurrent maintenance of antigen-specific IgG1 during AHI. Moreover, we found that a sequential appearance of gp41-specfic IgG3 followed by p55- and gp140-specific IgG3 which is consistent with our previous findings involving overall HIV-specific IgG responses (A22). These data support that inclusion of qualitative measurements (yes/no for the presence of certain antibody specificities) and quantitative measurements will be informative for determination of the relative timing post HIV-1 transmission.

New Discovery HIV-1 Epitope Mapping

In our studies of the antibody response in acute infection, we have found that antibodies to particular epitopes within gp41 and gp120 Env appear first. The immunodominant region in gp41 and the V3 region in gp41 are the first antibody responses to appear. As shown in FIG. 1, antibody responses to the MPER and the CD4 binding site (CD4bs) appear later. We have published recently using new technology that can quickly map plasma samples using an HIV Env peptide library containing 15-mer peptides that cover the full length of the consensus HIV Env gp160 sequences for Clade A, B, C, D, Group M, and CRF (circulating recombinant form) 1 and CRF 2 with >1000 peptide sequences in a single run. We have successfully applied this technology to examine mucosal specimens (A6, A20) and to identify novel epitopes to include for vaccine correlates analyses (A9). We are using this epitope mapping in a novel strategy to map epitopes for IgG3 and IgA and IgG1 across HIV proteins Gag and RT in addition to Env. As the antibody response matures over the course of the first year in response to virus evolution, we can include these unique antibody specificities/avidity measurements as part of the incidence testing. Thus, in addition to the decay of certain antibody types (ie. Gp41 IgA mAb, IgG3 Gag), the combined measurement different epitope specificities and avidity occurring later in the first post infection has a high likelihood of extending the mean duration of recency near 12 months.

IgA Measurements

We examined matching plasma and mucosal samples from Clades B and C for IgA and IgG responses to HIV-1 gp41 and used exponential models to determine the half-life (days) and concentration (μg/mL) at peak, half-life and asymptote (lowest estimated concentration value) (Yates N, Nolan T, Vandergrift N, Stacey, A, Borrow, P, Moody A, Montefiori D, Weinhold K J, Blattner W A, Shattock R, Cohen M, Haynes B F, Tomaras G D. HIV-1 Envelope IgA is Frequently Elicited after Transmission but has an Initial Short Half-Life. (2013) Nature Mucosal Immunology, epub January July; 6(4):692-703). The half-life of plasma gp41-specific IgA 48.19 days (95% CI=34.57-61.81)) and the half-life of mucosal IgA was 2.71 days (95% CI=2.06-3.36). The fold decline (the delta from peak to nadir) of HIV-1 specific IgA was similar in mucosal (6.20-fold (95% CI=−0.51, 12.92) and plasma (8.65-fold (95% CI=3.38-13.93) samples. Analysis of the decline in gp41 specific IgA responses during acute HIV-1 infection, in contrast to the stable levels of IgG gp41 indicate that this may be a useful measurement for an incidence test. Moreover, IgA responses to other regions of HIV-1 develop later in infection. In particular gp120 and p31 IgA responses (with concentration and avidity and specific epitopes within these. We will measure the epitope specific responses within gp41 and determine the decline in IgA in a larger set of plasma samples in different clades and in comparison to chronic infection to further refine the estimates as listed in Table 3.

The half-life estimates and concentrations of anti-gp41 IgA antibodies systemically and in genital fluids during acute infection, as reported here, are additional measurements that have potential as part of an HIV-1 incidence algorithm. There is precedence for the utilization of pathogen-specific IgA in tests for recent infection, such as in the case of dengue (16), that utilizes the short half-life of IgA for determining recent infection. Additional characterization of HIV-1 specific IgA responses at later stages in HIV-1 infection is needed to determine whether the IgA profile can distinguish recent from chronic HIV-1 infection.

Data show that serum dimeric IgA (dIgA) can be detected using novel antibody-detection reagents based on the inherent properties of the polymeric immunoglobulin receptor (pIgR), while secretory IgA (SIgA) can be detected with anti-pIgR antibodies. A chimeric rabbit/human pIgR (R/HpIgR) is expressed at high levels in cell culture and allows preferential detection of the relatively low amounts of dIgA present in blood. One hypothesis to be tested as part of this proposal is that measurement of SIgA, dIgA1, dIgA2 could distinguish recent infection. We routinely deplete IgM using specific column purifications before the measurement of secretory and dimeric IgA to ensure specific detection of IgA. It is hypothesized that acute HIV infection leads to local HIV-specific dIgA1 and dIgA2 responses in the gut mucosa and transcytosis to form SIgA in the gut, but the reduction in gut barrier function (A4) results in significant SIgA leakage into blood. SIgA has a longer half-life than dIgA and would be detectable for at least 6-12 months, providing a potential marker of incident HIV infection.

Measurements of IgA half-life can be used to understand the HIV-1 specific IgA concentrations that occur during the mean duration of recency. We then propose that in a single sample, we could relate the measured gp41 HIV-1 IgA concentration, to other IgA, IgM and IgG HIV-1 antibody response that together would provide information on recency.

We studied in detail the initial mucosal antibody response to gp41 (Yates, Tomaras et al. In Revision, Nature Mucosal Immunology 2012) and found that although the initial response declines rapidly, IgA is detectable and IgA specificities to other HIV-1 antigens appear later. Using our multiplex antibody assays, we measure concentrations of antibodies and proposed to relate also the relative amount of specific antibody in addition to the presence/absence of specific antibody. Moreover, in recent studies, we found robust detection of antibodies in saliva, and believe that these may be more reflective of serum antibody levels and provide a longer mean duration of recency to utilize specific IgA measurements.

We previously published that IgA antibody detection method is specific for IgA and not IgM (Tomaras et al. J. Virology 2008). For the IgA assays to test the concept of detecting polymeric forms of IgA during the acute stage of infection, the samples will first be depleted of IgM, using routine methods, in order to specifically detect IgA.

Measurement of the Maturation of the B Cell Response to HIV-1

In contrast to the IgA and IgG3 declining responses, the anti-Env response, gp41 and gp140 increase in avidity over the course of acute infection. Gp41 HIV specific IgG antibodies exhibit increasing avidity within approximately the first 30-40 days after transmission (Liu, et al. in preparation). Increasing antibody avidity is a marker of affinity maturation of the B cell response to a defined antigen. We have developed an avidity index assay based on the BAMA assay (BAMA-AI), which enables the measurement of avidity of antibodies in plasma, saliva or urine to multiplex different antigens and epitopes simultaneously. This assay builds upon existing avidity index assays (BED, AxSYM avidity, LAg, and Architect Avidity), but adds multiplex capability to measure both antibody titer and function to several key epitopes in a simultaneous, high-throughput analysis of a single assay.

Any suitable platform that permits antibody antigen detection, quantitative or semi-quantitative, could be used to determine Ab/Ag interactions, and/or avidity measurements. Non-limiting examples, include ELISE, lateral flow based platforms, platforms where Ag is immobilized and the samples are run and the like.

The invention contemplates use of any suitable biological sample, including but not limited to blood, plasma, serum, urine, saliva, mucosal samples, and so forth.

We have tested a panel of 101 HIV+ subjects (acute and chronic), and compared avidity index for the gp41 ID epitope. Excellent correlation was obtained between the two platforms (Spearman r=0.83, p>0.0001) (Seaton, Tomaras et al. Keystone HIV-1 Vaccine Meeting 2012)(23). The customizable multiplex capability of the BAMA provides an additional advantage in that it is readily adaptable to antigens or antibody subclasses of interest, providing flexibility as novel antibody parameters are defined and integrated into new testing algorithms. Thus, our system is capable of multiplexing antibody concentration, specificity and avidity.

We have significant data on antibody dynamics in acute/recent infection and have developed three assay technologies custom for HIV-1 antigens that provide highly sensitive detection of low levels of antibodies, capability of assessing multiple epitopes/antigens simultaneously, includes measurements of avidity in the same assay as concentration and ability to detect new HIV-1 epitopes that could increase accuracy in defining recent from chronic infection. Moreover all of this can be ascertained for different antibody isotypes and subclasses, in particular IgG3, IgA, IgM and IgG1 and in different sample types (plasma/serum, saliva, urine). The level of sensitivity of the assay at <1 ng/ml for defined HIV-1 epitopes indicates that low levels of antibodies even in controllers or AIDS patients or those on therapy could have detectable levels of specific antibody isotypes that would score in a sensitive multiplex assay as chronic, as opposed to a false recent. The ability to determine avidity to different HIV-1 proteins and epitopes for IgG3 and IgA as opposed to just total Ig will increase accuracy for defining later stages of infection.

The primary goal of this study is to utilize a single cross sectional-sample for this assessment without the need for a confirmatory assay. Plasma/serum and saliva the primary specimen since all of the patient populations with different characteristics have this specimen type available. The factors that can influence the measurements of the immune response to HIV-1 infection include HIV-1 subtype, antiretroviral use, low CD4 T cell counts, and natural suppression of HIV-1 due to virus fitness and/or host immune responses.

Measurement of biomarkers in cross-sectional samples would be facilitated by the use of biological specimens that are easy to collect, such as saliva or oral fluid. We have found HIV-1 antibody responses (gp41 MPER and immunodominant gp41) in saliva from subjects from the Duke Adult ID Clinic using the HIV-1 binding antibody multiplex assay and can explore the feasibility of saliva and oral fluid for use in incidence testing. HIV-1 specific antibody responses are detectable in oral fluid (10) and this is the strategy for OraQuick ADVANCE HIV-1/2 Antibody Test (OraSure Technologies, Inc.), thus determining whether the specific antibody measurements as defined by this project are present in detectable and predictable levels in this easy to obtain fluid will be critical. To date, we have saliva samples that cover both acute and chronic infection (Table 3) with planned sample collection for Protocol 217 that could include collection of oral fluid (MHRP), in addition those specimens to be tested from CEPHIA.

In certain aspects, the invention provides methods and reagents to determine which antibody subclasses and forms (IgG3, IgG1, IgA, dIgA, SIgA) of multiple antigen/epitope specificities and measurements (presence/absence, concentration, avidity) in the same assay test can distinguish recent from chronic infection. The invention also provides methods and reagents to determine the influence of population characteristics (e.g. multiple clades, ART vs. naïve, low CD4 T cell count, virus controllers) on the ability of these measurements to distinguish recent from chronic infection.

In certain embodiments, the methods are based on antibody measurements that can be tested as part of a multi-parameter test within a single sample. We will test the new concepts of temporal concentrations of plasma IgA (dIgA and mIgA), test novel epitope specificities for different antibody subclasses, isotypes, and test the accuracy of multiplexed antibody measurements measured simultaneously in an easy-to-obtain specimen type (for example plasma/serum, saliva/oral fluid, urine, or any other biological sample). We will extend and validate our studies on determining the temporal concentration, specificity and avidity (acute to recent to chronic) of HIV-1 specific IgG3 (gp41, Gag, RT) and (g41) IgA. Our approach for this first objective involves determining whether any of the antibody tests in isolation or in combination (multiparameter test) can appropriately classify the currently known misclassified samples as part of CEPHIA. This is a targeted and rapid approach that will allow us to quickly rule out specific antibody measurements and identify those tests to refine and develop that are substantially more accurate than current measurements. We will define whether any of these immune assays (singly or in combination) accurately classify samples (from CEPHIA) that are known to be misclassified by currently available incidence assays. We will examine a broader range of specimens from multiple clades and regions (Clade A, AE, C, G, etc.) and ART status to define concentration of these antibody specificities and determine the universal parameters that distinguish recent from chronic infection. Our output measurement used to determine HIV-1 incidence in a sample will include quantitative and qualitative assessments and include antibody avidity measurements of each of the antibody specificities.

Antibody Measurements

The types of immunological assays to be examined are: 1. antigen/epitope; and/or 2. antibody avidity/affinity, and/or 3. antibody isotype. We have several different variables that we propose are immediately testable for their ability to stage a sample as “recent” or “chronic”. The presence or absence, concentration, and/or avidity index of specific HIV-1 antibody responses (for example but not limited to gp41 IgG3, p55 Gag IgG3, p66 RT IgG3, gp41 IgA, gp41 IgM, as well as IgG, IgG1, IgG3, IgG4, IgG2, dIgA, mIgA, SIgA to envelope gp120 and gp41, protease, Gag, Integrase, and Rev, or any combination thereof will be determined. We will utilize multiplex luminex antibody assays, avidity measurements by multiplex bead assays and HIV-1 peptide microarrays. We will use peptide microarrays to identify new epitope specificities for IgA and IgG3 (and IgG1) that may be novel targets.

HIV-I Multiplex Binding Antibody Assays

Customized multiplex HIV-1 binding assays were performed as previously described (Tomaras et al, J Virol 82:12449-12463 (2008)) to determine IgG1 and IgG3 responses specific for recombinant HIV-1 p55 Gag (Protein Sciences, Meriden, Conn.), recombinant HIV-1 gp41 MN (Immunodiagnostics, Woburn, Mass.), a previously-described artificial multi-clade group M consensus gp120 Env protein (Con6 gp120) (Drs. Liao and Haynes, Duke University) (Gao et al, J Virol 79:1154-1163 (2005)), HIV-1 p66 reverse transcriptase (RT) (Protein Sciences, Meriden, Conn.), HIV-1 recombinant Nef (Genway, San Diego, Calif.), recombinant HIV-1 Tat (Advanced Bioscience, Kensington, Md.), and recombinant HIV-1 p31 Integrase (Genway, San Diego, Calif.) (Tomaras et al, J Virol 82:12449-12463 (2008)). HIV-specific antibody subclasses were detected with mouse anti-human IgG1 (BD Pharmingen) and mouse anti-human IgG3 (Calbiochem) on a Bio-Plex instrument (Bio-Rad, Hercules, Calif.) and μg/ml equivalents were calculated using a 4PL curve analysis with IgG subclass standards. Mouse anti-human IgG1 and IgG3 detection antibodies were tested for cross-reactivity to IgG1, IgG2, IgG3, and IgG4 and found to be highly specific for subclass detection. HIVIG (Quality Biological) and a constant HIV+ serum titration were utilized as positive controls and negative controls were included in every assay. All assays were run under GCLP compliant conditions, including tracking of positive controls by Levy-Jennings charts. Positivity criteria (mean MFI+3 STDEV) for antibody-antigen pairs were predetermined using a set of plasmas from 30 seronegative subjects. FDA compliant software, Bio-Plex Manager 5.0, (BioRad, Hercules, Calif.) was utilized for the analysis of specimens.

Various embodiments of the incidence biomarkers and methods described herein provide several advantages and distinctions over the prior art. Without limitations, these include: determination that a set of 4 antigen/antibody measurements is a suitable number of measurement to maximize MDRI and minimize FRR. A set of 4 measurements improved MDRI and FRR compared to currently used assays. Using >4 measurements in the algorithm did not significantly improve MDRI or FRR, and <4 measurements did not provide sufficient sensitivity to discriminate between recent and longstanding infection. Another improvement of the inventive markers and methods compared to currently available assays is the use of samples on ART during the algorithm training phase. The field currently excludes samples on ART from overall measurements of MDRI and FRR due to high FRR in treated populations. In developing the instant biomarkers and methods, ART samples were used to train the algorithm, and this greatly reduces the FRR overall and enables expanded use cases in populations with access to ART.

In one embodiments, the solution employs 4 biomarkers utilizing Envelope sequences. Currently available assays with multiple biomarkers use a combination of Env and non-Env antigens.

In some embodiments, the methods utilize IgG3 and IgG4 specific detections, which are not specifically used in currently available commercial assays. Some of the current assays use detection antibodies that capture all IgGs; where the current biomarkers and methods are unique in that it a specific IgG subset is analyzed.

Two of the solutions identified are distinct from each other in that one of the solutions is weighted toward longstanding markers, while the other solution from the Pitt Panel 2 is weighted more toward recency markers.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

As will be apparent to one of ordinary skill in the art from a reading of this disclosure, the embodiments of the present disclosure can be embodied in forms other than those specifically disclosed above. The particular embodiments described herein are, therefore, to be considered as illustrative and not restrictive. Those skilled in the art will recognize, or be able to ascertain, using no more than routine experimentation, numerous equivalents to the specific embodiments described herein. The scope of the invention is as set forth in the appended claims and equivalents thereof, rather than being limited to the examples contained in the foregoing description.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Specific Antibody Measurements can Accurately Determine Recent HIV-1 Infection

This example provides various methods and reagents to determine the temporal concentrations and avidity of multiple HIV-1 specific IgG subclasses and IgA forms and specificities in plasma, serum, saliva, and urine.

Activity 1 Provided are methods and reagents to determine if presence and level of dimeric IgA in plasma provides information on recent infection

Results/Milestones

Determine if IgA detection reagents can sensitively and specifically detect IgA forms exclusive of IgM.

Determine the antibody dynamics of HIV-1 specific IgA forms during recent infection in longitudinal plasma samples.

Determine if a specific HIV-1 IgA form and output measurement (qualitative, quantitative and and/or avidity) can distinguish between recent and chronic infection.

Activity 2 Provided are methods to identify novel epitopes in Env, RT and Gag for IgG, IgG3 and IgA that distinguish recent and chronic infection.

Results/Milestones

Map dynamic linear epitope specificities of HIV-1 specific IgG3 and IgA antibodies in longitudinal samples.

Determine whether new epitope specificities with particular output measurements (qualitative, quantitative and and/or avidity) can distinguish recent from chronic infection.

Activity 3 Provided are methods to determine if the selected HIV-1 epitope specificities, antibody form and output measurements can classify misclassified samples as part of CEPHIA.

Results/Milestones

Select combinations of antibody forms, specificities and output measurements in an iterative fashion for testing whether a combination improves classification of recent infection above that of single measurements and outputs in cross-sectional samples of defined recent and chronic infection.

Determine if combining the selected antibody specificities and output measurements from Activities 1 and 2 improve classification of recent infection above that of single measurements in known misclassified samples as part of CEPHIA.

Identify unique antibody/antigen pair and output measurement that demonstrates superiority above other measurements in accurately classified previously identified “misclassified samples”.

Activity 4 Determine the applicability of identified antibody measurements to saliva and/or urine, or any other suitable biological sample.

Results/Milestones

Determine if a downselected HIV-1 IgA form and output measurement (qualitative, quantitative and and/or avidity) chosen from Activity 1 can distinguish between recent and chronic infection in saliva and/or urine.

Determine if downselected epitope specificities with particular output measurements (qualitative, quantitative and and/or avidity) chosen from Activity 2 can distinguish recent from chronic infection in saliva and/or urine.

Determine if the downselected combined antibody specificities and output measurements from Activities 3 improve classification of recent infection above that of single measurements in saliva and/or urine.

Results Measurement/Analysis Plan

For this objective, we will combine multiple antibody measurements (including detection and concentration) to classify recent HIV-1 infection. We propose that a more accurate HIV-1 test based on antibody measurements can be obtained using multiple antibody responses assessed simultaneously in a single sample rather than by considering only an individual antibody response. Since the goal is to obtain a measurement which is accurate and robust across populations, the model for classifying individuals as recent infections (the first requirement above) based on multiple immune responses will necessarily need to be parsimonious. We plan to use a combination of variables, both quantitatively and qualitatively for determination of a recent infection. To determine the robustness of an assay measurement we will examine each measurement alone for the ability to distinguish recent from chronic infection and then will apply the most robust measurements to testing misclassified samples. This is different from current algorithms in that here we are testing which antibody and antigen pairs within a single assay type can be used together to improve accuracy of classifying recent infection above existing strategies.

For each measurement (e.g., concentration of gp41 IgG3), the temporal dynamics will be characterized. For the measurements that are successful at classifying and distinguishing recent from chronic infection, we will begin to combine the measurements that are in the multiplex assay. As the immune responses between individuals are quite variable, having a combination of antibody measurements, each with different weights will make the staging prediction more accurate. We propose to apply different weights to each of the tests based on the time post transmission that they arise at the population level. For each measurement (e.g., concentration of gp41 IgG3), the temporal dynamics will be characterized. In particular, longitudinal trajectories will be modeled using exponential decay models (A25) fit to repeated antibody measurements using non-linear mixed effects software such as Proc NLMIXED in SAS. For antibody measurements that are quantitative (such as peak concentration or avidity), receiver operator characteristic (ROC) curves will be estimated in order to determine thresholds for classifying individuals as acutely infected. Estimated ROC curves will be compared across antibody measurements and across populations to determine which individual measurements most accurately and consistently predict whether individuals are recently infected. More modern classification procedures such as (supervised) machine learning or neural networks can also be employed in this setting. We will employ logistic regression, machine learning, and neural networks to develop several candidate classification algorithms.

We will use a substantial portion of the data (the “training set”) and then assess the accuracy (i.e., sensitivity and specificity) of the algorithm based on the remaining data (the “test set”). The disadvantage of such a “Train-and-Test” approach is a lack of efficiency, as the testing portion of the data (typically 20-30% of the total data set) is not used in selecting the algorithm. In large data sets this lack of efficiency is not necessarily of concern. However, in the current setting the sample sizes are more moderate. Therefore, cross-validation (a variant of the “Train-and-Test” approach) or the bootstrap procedure will be employed to estimate an algorithm's sensitivity and specificity.

For those assay measurements that are successful in identifying recent from chronic infection, we will test them on the set of samples as part of CEPHIA that are misclassified (false recent). We will use the lower bound of a 90% exact binomial confidence boundary (a one-sided alpha 0.05) to provide a conservative estimate of the improvement in the false recent rate for the new assay. Given the improved false recent rate, we will perform a superiority test of the new method to the old method in terms of false recent rate. A one-side binomial test at the alpha 0.05 level will be used to conduct a superiority test of the new false recent rate to the old rate. For example, if we can test 60 misclassified samples, we would have 84% power to determine an improvement of the false positive rate to 7.9%, if the new assay truly detects chronic samples for 55% (A33) of the 60 misclassified samples, then the average lower bound of a 90% exact binomial confidence limit (based on a simulation of 2000 iterations) will be 43.8%, which is an improved false recent rate of 7.9%.

HIV-1 Incidence Measurements

Antibody Multiplex Array.

Maturation and kinetics of HIV-1 antibody isotypes and subclasses with recognition of different components of HIV-1 can distinguish recent infection. Multiplexed high data content assays allows use of single assay with single small volume sample. Identify novel antibody biomarkers of HIV-1 incidence.

In combination, the presence, magnitude, ratio and avidity of multiple different antibody isotypes, subclasses to different HIV-1 proteins and epitopes can differentiate recent from chronic HIV infection.

Antibody Multiplex Array Rationale.

(1) Antibody Specificity: Sequential appearance of HIV-1 IgG Specificities; and Differential Durability of Gag and Env. (2) Antibody IgG Subclass: IgG3 antibodies to gp41, Gag and RT are frequently induced in acute HIV-1 and decline with defined kinetics during acute HIV-1 infection; and Delayed kinetics of IgG4 responses. (3) Antibody IgA Isotype: Sequential appearance of HIV-1 IgA Specificities; and Antibody Forms (e.g. dIgA). (4) Antibody Avidity: Affinity maturation occurs for each epitope specificity.

New IgG3 and IgG Epitope Mapping Env, Gag, and Pol in Acute-Chronic Infection.

Full length sequence of HIV Gag p17 and p24, Tat, Nef and Env plus remaining immunogenic regions of HIV (Pol, Rev, Vif). (Clades A, B, C, D, G, CRF1, CRF2). >200,000 Ab-antigen measurements determined for 4-6 patients at recent and chronic infection time points. We selected 5 new epitopes from this mapping strategy that best discriminated recent from chronic infection for further analysis. New epitopes identified in Env, Gag, Protease, Integrase, Rev.

Data Analyzed CEPHIA Developmental Panel.

281 Antibody-Antigen Measurements:

IgG: 21-29 antigens (protein+peptide); IgG3: 13 protein antigens; IgG4: 16 protein antigens; IgA: 21-29 antigens (protein+peptide); and dIgA: 24 antigens (protein+peptide).

Output measurements: Magnitude (MFI), Dilution and Avidity

CEPHIA Developmental Panel (75 ARV-Naïve Samples):

35 HIV+, >12 months; 16 HIV+, infected <6 months; 22 HESN; and 2 HESN/Acutes.

Statistical Methods.

Antigen selection: Logistic model for each antigen predicting longstanding vs. recent; Panel 1 contained Selected antigens based upon χ2>9 and limiting to one antigen per panel. K=9. Panel 2 contained Selected antigens based upon the best χ2 per isotype/subclass. K=6. Discriminant function analysis was performed.

Further Experimental and Analytical Methods.

Analyze the full data set by with increased N (Currently have 46 additional recents and 34 chronics) by discriminant analysis. Test the optimal sets of antigen/Ab panels and newly identified epitopes on an increased N with each correct classification group. Examine other analytical approaches (i.e. Structural Equation and Finite Mixture Model).

Summary.

We identified new IgG and IgG3 epitopes (Env, Protease, Integrase, Gag, Rev) in acute infection with potential to discriminate acute/chronic infection. We found that multiple forms of IgA, including dIgA (gp41, Gag) appear in acute infection. This biomarker, dimeric IgA, (pIgR detection) was advanced for testing in CEPHIA panels. Our test of the first CEPHIA panel demonstrates that we can classify recents and chronics using a panel of Ab-Ag measurements including multiple antigen specificities and antibody subclasses/forms.

### Example 2: Harnessing Antibody Responses as HIV-1 Incidence Biomarkers

This example shows methods how to identify novel antibody biomarkers of HIV-1 incidence.

WHO-UNAIDS 2013 Update on HIV-1 Incidence Assays:

None of the current assays are ideally suited for global HIV-1 incidence tested; False-recent rate in long term infections due to: elite control, ART, advanced AIDS, and variations across individuals, populations, geographic regions and virus subtype.

CEPHIA (Consortium for the Evaluation and Performance of HIV-1 Incidence Assays):

Evaluated BED CEIA assay, Limiting antigen (LAg) Avidity EIA, Vitros, Less Sensitive and BioRad Avidity Index EIA. However, none met the target product profile for an incidence assay.

Innovative Strategies to Develop a New Global HIV-1 Incidence Assay:

Goal is to achieve the Target Product Profile (TPP); TPP: Mean duration of recency (6-12 months); single assay; appropriate for all clades, easily obtainable sample, etc.

HIV-1 Incidence Assay Strategy—Antibody Array.

Maturation (avidity) and kinetics of HIV-1 antibody isotypes (IgA forms) and subclasses (IgG3) with recognition of different components of HIV-1 (gp41, RT, Gag, gp120) (likely resulting from the early depletion of CD4+ T cells and destruction of germinal centers in acute infection) can distinguish recent infection. Multiplexed high data content assays allows use of single assay with single small volume sample.

Antibody Array Rationale

HIV-1 infection induces a series of antibody specificities and antibody forms (isotypes, subclasses) that have differential kinetics over the course of the first year of infection. IgG antibodies to gp41 arise first, followed by Gag, and gp120 specificities. Epitope specificities within gp120 also arise sequentially with V3 arising first, followed by MPER, CD4 BS etc. (Tomaras J V 2008). Antibody responses to the HIV-1 env is regulated differently from antibodies to Gag, with Gag specific antibodies being maintained longer than Env antibodies during ART treatment. Antibody affinity maturation occurs can for each antibody specificity over the course of infection. IgG3 antibodies to gp41, Gag and RT are frequently induced in acute HIV-1 and decline with defined kinetics during acute HIV-1 infection (Yates, Tomaras AIDS 2010). For the IgA response, gp41 is also the initial target with Gag IgA also arising early. IgA antibodies to gp120 and p31 are lower in frequency in acute infection. (Yates, Tomaras Mucosal Immunology 2013). Transient production of dIgA during the acute phase of infection may occur. For the IgG subclasses, IgG1 and IgG3 arise initially in most HIV-1 infected patients with a delayed kinetics of IgG4 responses. Breadth of antibody responses to gp120 increases over the course of HIV-1 infection.

Incidence Antibody Array

In combination, the presence, magnitude and avidity of multiple different antibody isotypes, subclasses to different HIV-1 proteins and epitopes can differentiate recent from chronic HIV infection.

Antigens:

Envelope (gp120, gp41), Gag, Integrase, RT, Nef, Tat, Rev proteins, peptides (V1V2 peptide in a non-limiting embodiment) and epitopes, and multiclade panels of gp120 and epitope specific responses. Non-limiting examples of antigens and epitopes include the Envelope 6043: RFPVPRGPDRPEGIE (720-734); Gag 2657: KIWPSSKGRPGNFPQ (436-450); Pol/Pro: 589: EEMSLPGRWKPKMIG (90-105); Pol/Integrase 1095: VYYRDSRDPLWKGPA (940-954); Pol/Integrase 1099: DSRDPLWKGPAKLLW (944-958); Rev: 396: PVPLQLPPIERLHLG (70-84).

Antibody Isotype:

IgG, IgA; Antibody Subclass: IgG1, IgG2, IgG3, IgG4, IgA1, IgA2; Antibody Forms: mIgA, dIgA, SIgA.

Output Measurements:

magnitude, avidity and Ab and antigen ratio.

Data Generated—CEPHIA Panel 1—281 Antibody-Antigen Measurements: IgG 21-29 antigens (protein+peptide); IgG3 13 protein antigens; IgG4 16 protein antigens; IgA 21-29 antigens (protein+peptide); dIgA 24 antigens (protein+peptide). Output measurements: 1) response magnitude (MFI), and 2) avidity. We selected antigens and epitopes from custom made gp120 and gp140 proteins that represent cross-clade envelope proteins (e.g. A1 Congp140, B Congp140, ConSgp140, Con6gp120, 1086 gp120 and 1086 gp140, and C1 and V1-V2 Antigens (Haynes B F, Gilbert P, McElrath M J, Zolla-Pazner S, Tomaras G D, Alam S M, Evans D, Montefiori D C, Karnasuta C, Sutthent R, Liao H, DeVico A, Lewis G, Williams C, Fong Y, Janes H, DeCamp A, Huang Y, Rao M, Karasavva N, Robb M L, Ngauy V, DeSouza M S, Paris R, Ferrari G, Bailer R, Soderberg K A, Andrews C, Berman P, Frahm N, De Rosa S C, Alpert M, Yates N L, Shen X, Koup R, Pitisuttithum P, Kaewkungwal J, Nitayaphan S, Rerks-Ngarm S, Michael N L, Kim J H. Immune Correlates Analysis of the ALVAC-AIDSVAX HIV-1 Vaccine Efficacy Trial, (2012) N Engl J Med. 2012 Apr. 5; 366(14):1275-86. PMID:22475592; Liao H, Bonsignori M, Alam S M, Tomaras G D, Moody M A, Tsao C, Hwang K, Lu X, Parks R, Montefiori D C, Ferrari G, Rao M, Karasavva N, McLellan J, Yang Z, Dai K, Pancera M, Rerks-Ngarm S, Nitayaphan S, Kaewkungwal J, Pitisuttithum P, Tartaglia J, Sinangil F, Nabel G, Mascola J, Kwong P, Kim J, Michael N L, Pinter A, Zolla-Pazner S, Haynes B F, “Vaccine Induction of Antibodies against a Structurally Heterogeneous Site of Immune Pressure within HIV-1 Envelope Protein Variable Regions 1 and 2” Immunity Volume 38, Issue 1, 24 Jan. 2013, Pages 176-186. PMCID: PMC3569735; Zolla-Pazner S B, deCamp A C, Cardozo T, Karasavvas N, Gottardo R, Williams C, Morris D E, Tomaras G D, Rao M, Billings E, Berman P, Shen X, Andrews C, O'Connell R J, Ngauy V, Nitayaphan S, de Souza M, Korber B, Koup R, Bailer R T, Mascola J R, Pinter A, Montefiori D, Haynes B F, Robb M L, Rerks-Ngarm S, Michael N L, Gilbert P B, Kim J H. Analysis of V2 Antibody Responses Induced in Vaccines in the ALVAC/AIDSVAXHIV-1 Vaccine Efficacy Trial, PLoS ONE 8(1): e53629. doi:10.1371/journal.pone.0053629; Tomaras G D, Ferrari G, Shen, X Alam S M, Liao H, Pollara J, Bonsignori M, Moody M A, Fong Y, Chen X, Poling B, Nicholson C O, Zhang R, Lu X, Parks R, Kaewkungwal J, Nitayaphan S, Pitisuttithum P, Rerks-Ngarm S, Gilbert P B, Kim J H, Michael N L, Montefiori D C, Haynes B F. Vaccine induced plasma IgA specific for the C1-region of the HIV-1 envelope blocks binding and effector function of IgG. (2013) PNAS, vol 110; number 22, 9019-9024.

Statistical Methods

Antigen selection: Logistic model for each antigen predicting longstanding vs recent. Panel 1: Selected antigens based upon: χ²>9 and limiting to one antigen per panel. K=9. Panel 2: Selected antigens based upon: The best χ² per isotype/subclass. K=6. Data were analyzed in discriminant function analysis.

Development of Further Experimental and Analytical Methods

Data generated on 46 additional recents and 34 chronics/controllers. Analyze these data and also further increase the sample size for each classification group.

The full data set will be analyzed (with increased N) by discriminant analysis. Test the optimal sets of 5-9, 6-9, 5, 6, 7, 8 or 9 antigen/Ab panels on a larger data set with each classification group.

Examine other analytical approaches (e.g. Structural Equation and Finite Mixture Model).

Table 1. IgA Responses to HIV-1 Proteins During Fiebig Stages I-VI.

Gp41 IgA is elicited first and gp120 IgA appears in more subjects later in infection. ((Yates N, Nolan T, Vandergrift N, Stacey, A, Borrow, P, Moody A, Montefiori D, Weinhold K J, Blattner W A, Shattock R, Cohen M, Haynes B F, Tomaras G D. HIV-1 Envelope IgA is Frequently Elicited after Transmission but has an Initial Short Half-Life. (2013) Nature Mucosal Immunology, epub January July; 6(4):692-703).

TABLE 1
IgA responses to HIV-1 proteins during
Fiebig Stages I-VI. Gp41 IgA is

[symbol]

| Fiebig | gp41 | Gag | RT | Nef | Gp120 | p31 |
|---|---|---|---|---|---|---|
| Stage | % | % | % | % | % | % |
| I/II | 26.1 | 8.7 | — | — | — | — |
| III | 50.0 | 21.4 | — | — | — | — |
| IV | 88.2 | 58.8 | 42.9 | 42.9 | 25.0 | 0.0 |
| V/VI | 97.5 | 87.5 | 86.7 | 57.1 | 37.9 | 14.3 |

[symbol]

indicates data missing or illegible when filed

TABLE 2
HIV-1+ cohorts

| | | | # ART | # ART |
|---|---|---|---|---|
| Region | Clade | Infection Stage | Naïve | Treated |
| US/Trinidad | Clade B | Acute-Chronic^{a} | 46 | 93 |
| US/UK | Clade B | Chronic | 52 | 44 |
| Malawi, South | Clade C | Acute-Chronic^{a} | 142 | 20 |
| Africa | | | | |
| Malawi, South | Clade C | Chronic | 171 | 62 |
| Africa | | | | |
| Tanzania, Kenya, | Clades | Acute-Chronica | >20^{c} | n/a |
| | A, B, C, D | | | |
| Uganda, Thailand^{b} | AD | | | |
| | Recombinants | | | |
| Tanzania/UK | Clade A/C | Chronic | 75 | 2 |
| US | Clade B | Chronic, CD4 | — | 38 |
| | | count <400 | | |
| US | Clade B | Chronic, Virus | >21 | — |
| | | Controllers | | |

Table 2. HIV+ Cohorts (>780 subjects)
*enrolled as acute and followed longitudinally through recent and chronic stages.
^{b}Enrollment expected to expand to Mozambique (Clade C) and Nigeria for Clade G and AG recombinants during the course of this project.
^{c}Active enrollment.

TABLE 3
Saliva/oral fluid available for testing

| Cohort | Subjects |
|---|---|
| Acute (Clade B/C) | 13 (6 longitudinal acutes) |
| Chronic (Clade B/C | 59 |
| Negative | 73 |
| Acute (Clades A, B, C, D, E | In enrollment as part of |
| AG, G anticipated) | Protocol 217 MHRP |

### Example 3: Development of an Assay for Incidence Measurement

This example harnesses antibody responses and CD4+ T Cell associated virus for use as HIV-1 incidence biomarkers

Antibody Multiplex Array.

Maturation and kinetics of HIV-1 antibody isotypes and subclasses with recognition of different components of HIV-1 can distinguish recent infection in a binding antibody multiplex assay (BAMA) with high data content.

Cell Associated Viral Load.

Progressive seeding of HIV-1 in phenotypically defined CD4+ T cell subsets (naïve and memory) occurs during recent HIV-1 infection; thus ratios of cell associated VL in different memory CD4+ T cells can distinguish recent infection.

CEPHIA Stage Gates (FIG. 9).

1) Candidate biomarker(s) associated with recent HIV infection, able to be reliably measured, potentially useful as classifier (“tuneable” threshold)

2) Classification threshold(s) with MDRI 4-24 months

3) Classification threshold(s) with FRR <15% and consistency of FRR between major subtypes

4) Assay Protocol, kit availability, potential for commercialization

5) MDRI of 4-24 months AND FRR not inferior to BED; supply training, equipment etc. for independent evaluators

6) Demonstrate advantage over existing assays/algorithms; meet TPP for at least 1 Use Case

Multiplex Antibody Array.

In combination, the presence, magnitude, ratio and avidity of multiple different antibody isotypes, subclasses to different HIV-1 proteins and epitopes can differentiate recent from chronic HIV infection.

Patient Cohorts.

(a) CHAVI 001 acute longitudinal samples 2-75 weeks post infection) Fiebig stages I/II to VI (Clades B and C) and chronic (N=47).

(b) CEPHIA Developmental Panel 2 (75 ARV Naïve, including HESN, recent and longstanding infection samples) (Clade B) (N=43).

(c) Pitt Panel 2 (Clades A, B, C: 21 Recent (<9 mos), 63 Longstanding; 29 ART.

Data Analyzed CEPHIA Developmental Panel.

281 Antibody-Antigen Measurements were made. Envelope (gp41, gp120), and Non-Env Antigens and peptide epitopes (including ID and new epitopes) were evaluated. IgG, IgG3, IgG4, mIgA, dIgA was determined.

Output Measurements:

Magnitude (MFI), Dilution and Avidity.

Statistical Methods (7 Methods of Downselection from 505 Unique Measurements).

1) Use each measurement as a continuous predictor of Classification in a logistic regression.

2) Use each measurement as a categorical predictor of Classification in a logistic regression.

3) Rank order measurements by the absolute value of the correlation with EDSC.

4) Rank order measurements by the mean difference between Classification.

5) Rank order measurements by the difference in positive response rate.

6) Rank order measurements by the median difference and absolute threshold.

7) Combine the order score for methods 1-6.

For methods 1-7, we selected the top 20 measurements. Discriminant function analysis on all possible combinations of measurement sets CEPHIA/CHAVI, sizes 3-8; Pitt Panel 2, sizes 3-6. Best measurement sets selected based on misclassification rate and FRR determined. Discriminant function value estimated from best set. Predicted duration of infection (PDI) estimated from EDSC and DV. The MDRI is the mean of the PDI.

Discovery Phase Results.

Table 4. below shows the CEPHIA/CHAVI discovery phase results where the recency threshold used to classify specimens in panels used to date was 12 months.

CEPHIA/CHAVI

| | Longstanding | Recent by | |
|---|---|---|---|
| | by new assay | new assay | Total |
| Nominally | 45 | 2 | 47 |
| Longstanding* | | | |
| Nominally Recent | 0 | 39 | 39 |
| Total | 45 | 41 | 86 |

Top solution: FRR: 2.3% Error Rate: 2.3%

Panel Characteristics:

CEPHIA/CHAVI CEPHIA (N=43) and CHAVI (N=47); Clades B and C, AR.

Data Analyzed Proof of Concept Panel (Pitt Panel 2).

505 Antibody-Antigen Measurements were made.

Envelope (gp41, gp120, gp140), and Non-Env Antigens were examined.

Peptide epitopes (including ID and new epitope discovery from peptide microarrays) were examined.

IgG, IgG3, IgG4, mIgA, dIgA, IgM were examined.

Output measurements: Magnitude (MFI), Dilution, and Avidity

DFA Algorithm Results, Pitt Panel 2.

We gathered the 4 measurement sets (N=3) from method 1 that had a misclassification rate of ˜3.2%. This rate was observed for 4, 5 and 6 measurement sets (4 chosen by principle of parsimony). There were 6 measurements that were present in any of the solutions with a misclassification rate of ˜3.2% (2 PTIDs misclassified). One solution did not misclassify any Longstanding as Recent.

Proof of Concept Phase Results.

Table 5 below shows the Pitt Panel 2 results where the recency threshold used to classify specimens in panels used to date was 9 months.

| | Longstanding | Recent by | |
|---|---|---|---|
| | by new assay | new assay | Total |
| Nominally | 42 | 0 | 42 |
| Longstanding* | | | |
| Nominally Recent | 2 | 13 | 15 |
| Total | 44 | 13 | 57 |

Top 3 solutions: FRR: 0%, Error Rate: 3.5%; FRR: 1.6% Error Rate: 3.2%; and FRR: 1.6% Error Rate: 3.2%

Panel Characteristics:

Pitt Panel 2 (Clades A, B, C: 21 Recent (<9 mos), 63 Longstanding; 29 ART.

Common Features of Solutions for CEPHIA/CHAVI and Pitt Panel 2.

Table 6 Below shows Common Features of Solutions for CEPHIA/CHAVI and Pitt Panel 2. The solution involved 3 different antibody types (subclasses/isotypes); Direct binding and avidity measurements; and Gp41, gp140 and non-Env proteins.

TABLE 6

| Cephia/CHAVI Panel | Pitt Panel 2 |
|---|---|
| 2.3% FRR, 12 mos | 0% FRR, 9 mos |
| ✓ | ✓ |
| ✓ | ✓ |
| ✓ | ✓ |

Duke Multiplex Ab Array.

Table 7 below shows a comparison to draft TPP.

| | | Acceptable | New Assay |
|---|---|---|---|
| Assay Attribute | Ideal Performance | Performance | Characteristics* |
| MDRI¹ | 365 days | 180 days | 166-356 |
| FRR² | 0% | 2% | 0-1.6% |
| RITA³ | Single assay | In combination with | Single assay |
| | | ≥1 other assay | Anticipated |
| Subtype | All major | Regionally specific | Subtypes A, B, C |
| applicability | | | demonstrated |
| | | | All Major Anticipated |
| Acceptable | Any practically | Frozen serum/plasma | Frozen serum/plasma |
| specimen types | feasible | | Demonstrated |
| | | | Saliva, Urine |
| | | | Anticipated |
| Minimum | 10 μl | 1 ml | 10 μl Anticipated |
| specimen volume | | | |
| Infrastructure | None | Centralized laboratory | Handheld Device |
| requirements | | facility | Anticipated, Centralized |
| | | | Laboratory Facility |
| | | | Demonstrated |
| Cost^{†} | <$3 USD | <$10 USD | Unknown |

¹Mean Duration of Recent Infection (average time spent ‘recently’ infected while infected <T)
²False Recent Rate (% of subjects infected for > T but classified as recent)
³Recent Infection Testing Algorithm (e.g. Ab avidity assay + viral load)
*demonstrated or anticipated
^{†}estimates only for illustrative purposes

CAVL: Cellular Viral Burden as a Marker of Infection Recency.

CD4⁺ T-cells are the primary host cell infected by HIV-1. Virus preferentially targets memory CD4⁺ T-cells. (Brenchley et al., JV 2004, Mattapallil et al., Nature 2005). Without being bound by theory, viral load within naïve cells increases as infection progresses and susceptible memory cells are depleted. The approach comprises using cell-associated viral burden among CD4⁺ T cell subsets to distinguish recent and chronic infection samples: (a) Established assay in SIV+non-human primate longitudinal PBMC samples; and (b) confirm and refine assay in HIV+ human PBMC samples. These studies will establish the relative dynamics of establishing infection in CD4 compartments.

Phenotype of CD4 T Cell Subsets Down-Selected for Incidence Assay.

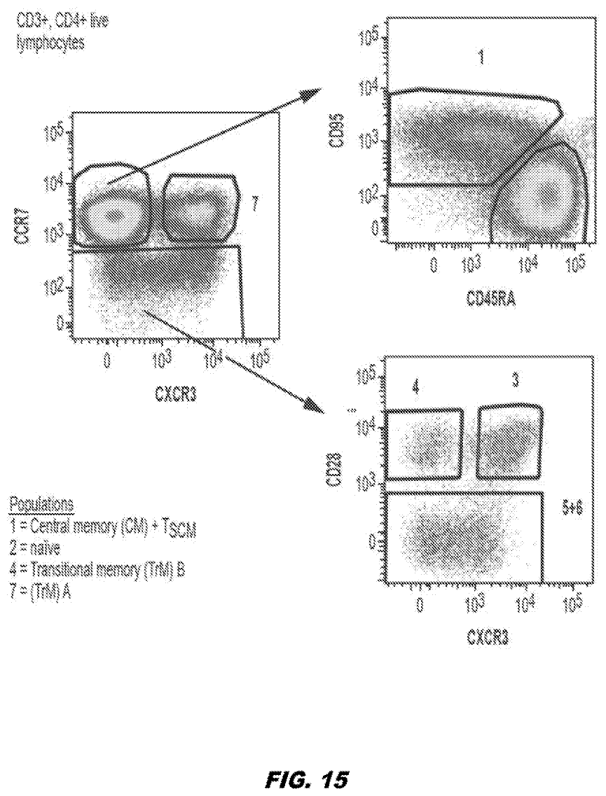

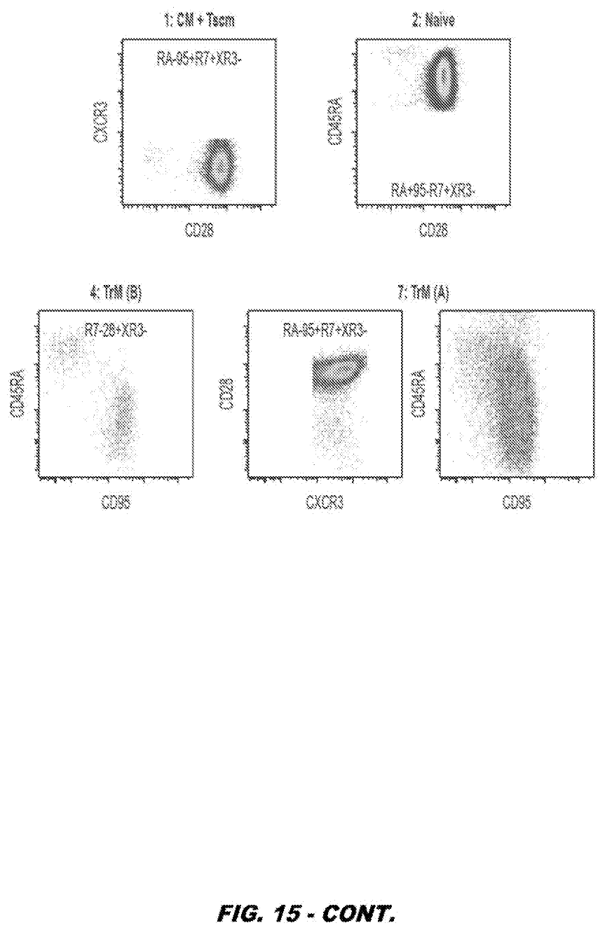

We separated CD4 T cells into different subsets based on the expression of memory cell markers, including CD28, 95, RA, and chemokine receptors. From our work in the rhesus macaque model, we down selected 4 populations as shown in FIG. 15.

Cell-Associated HIV-1 DNA.

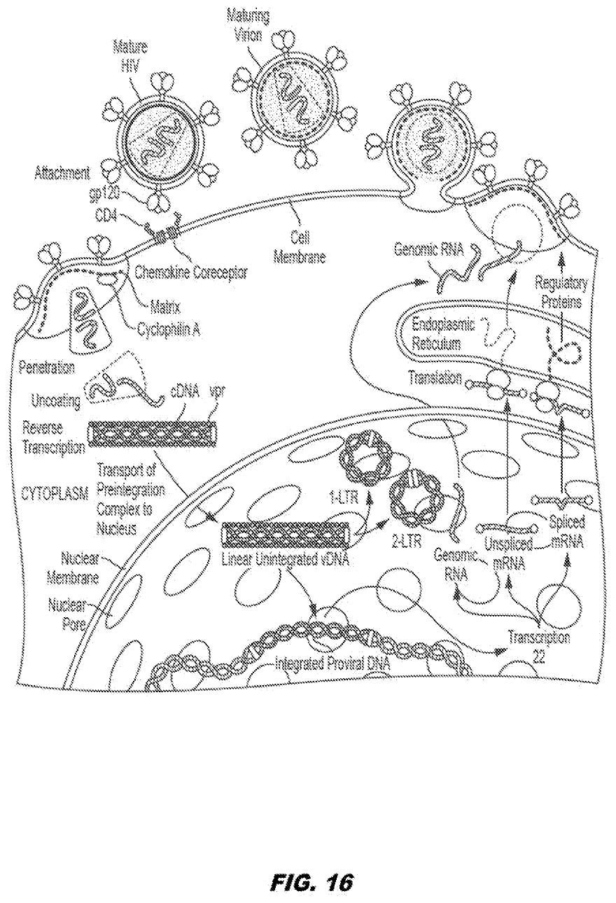

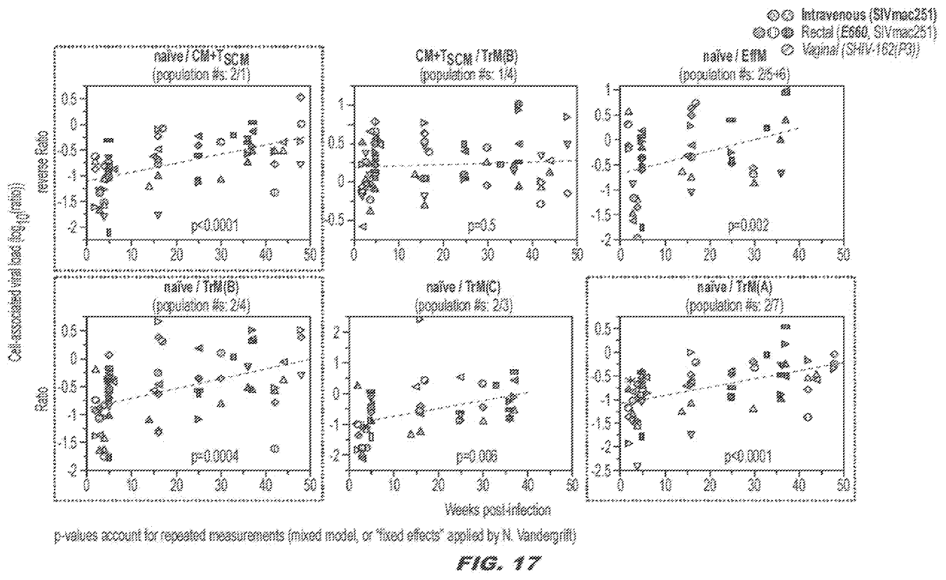

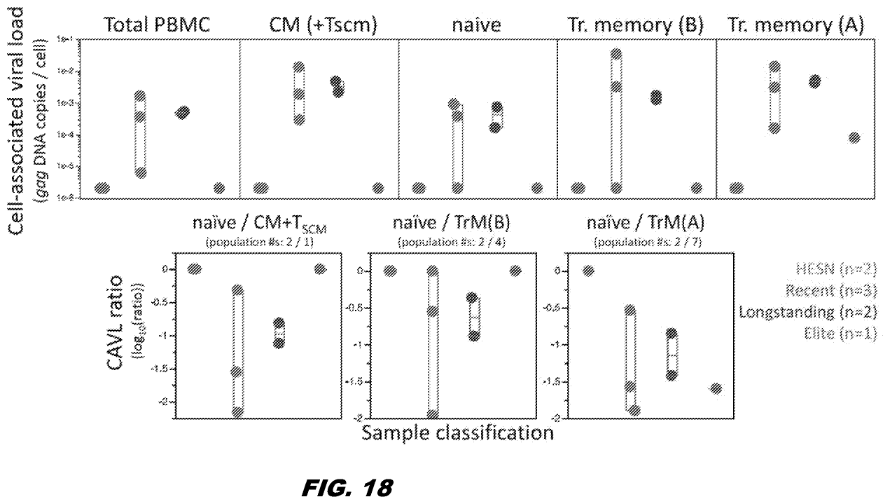

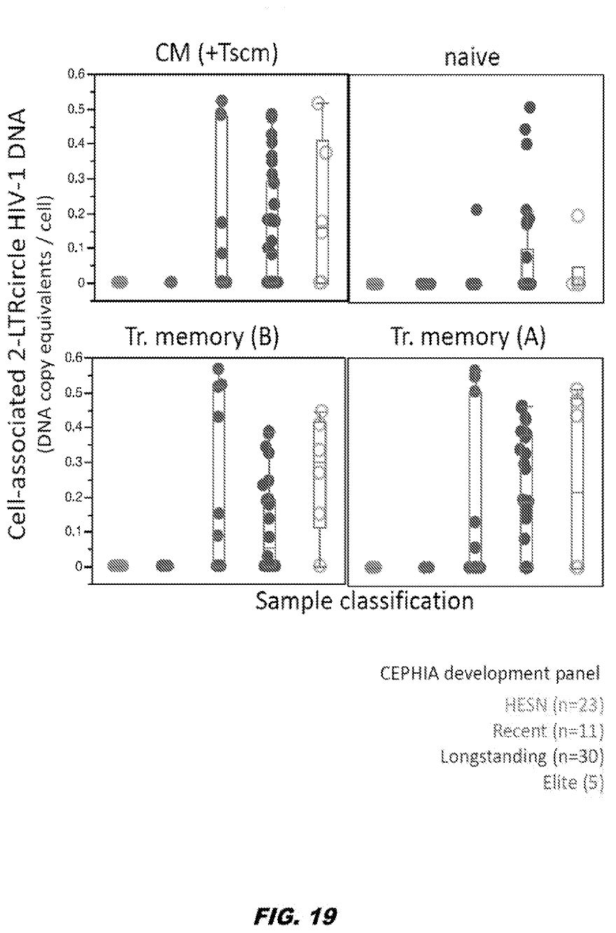

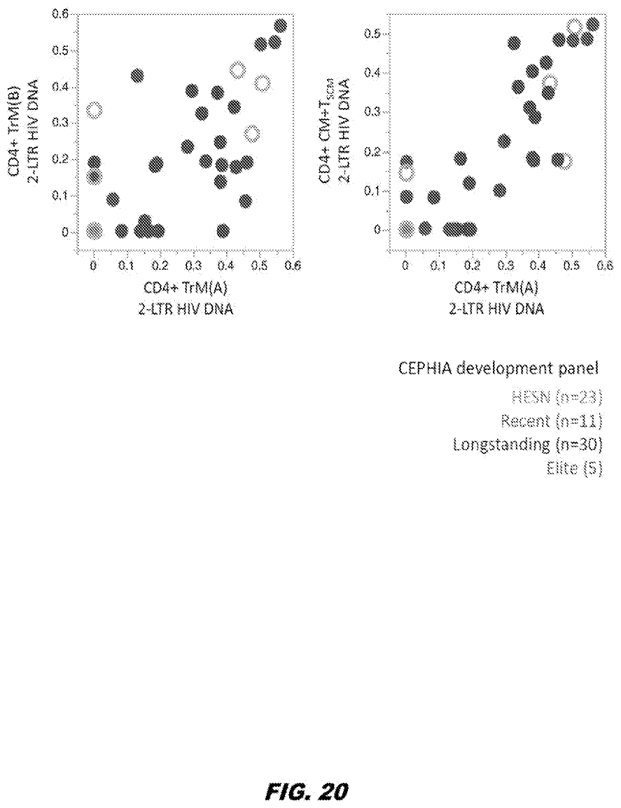

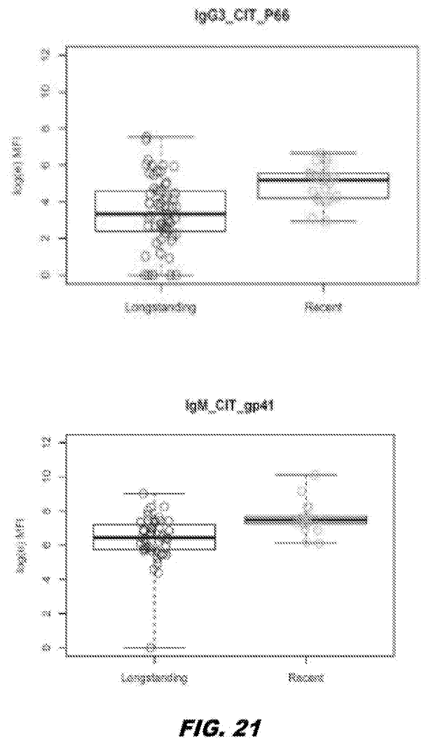

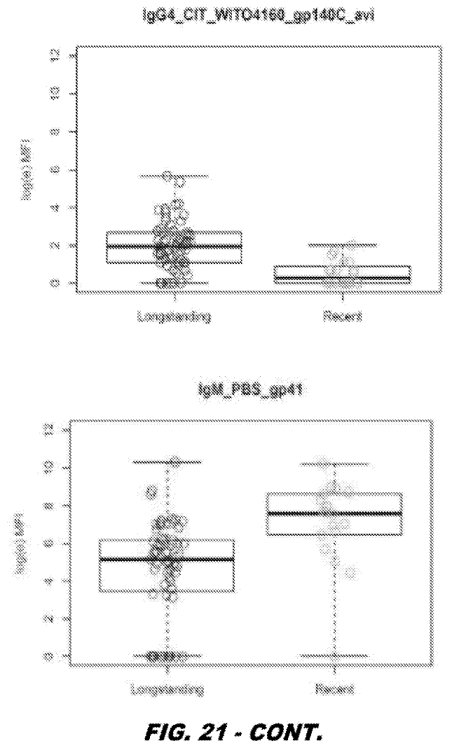

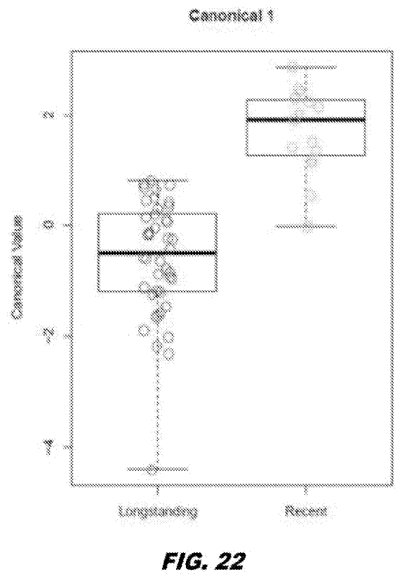

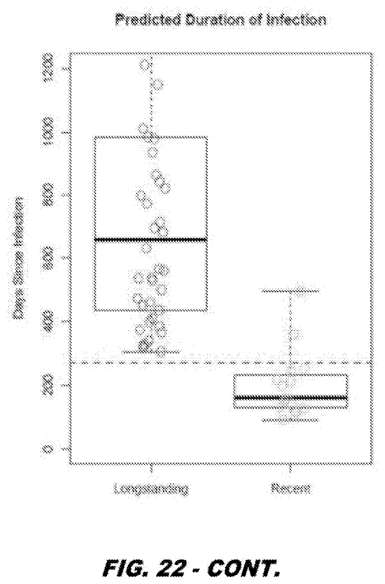

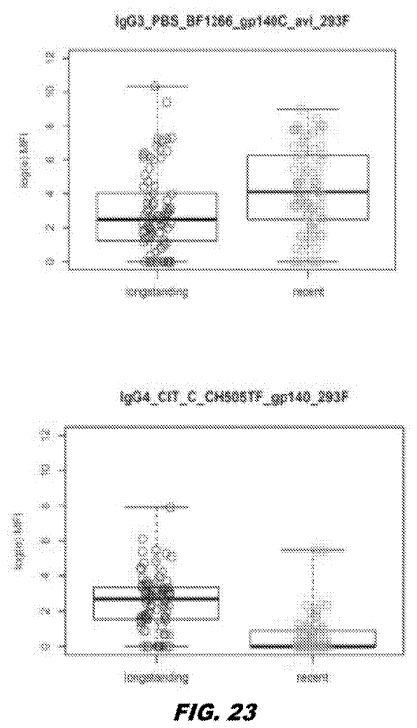

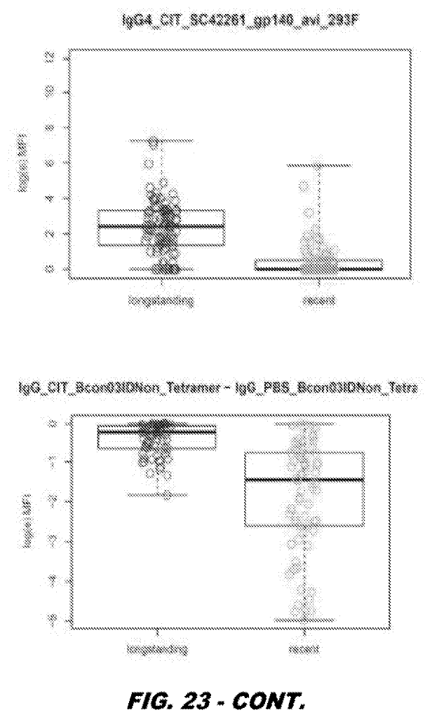

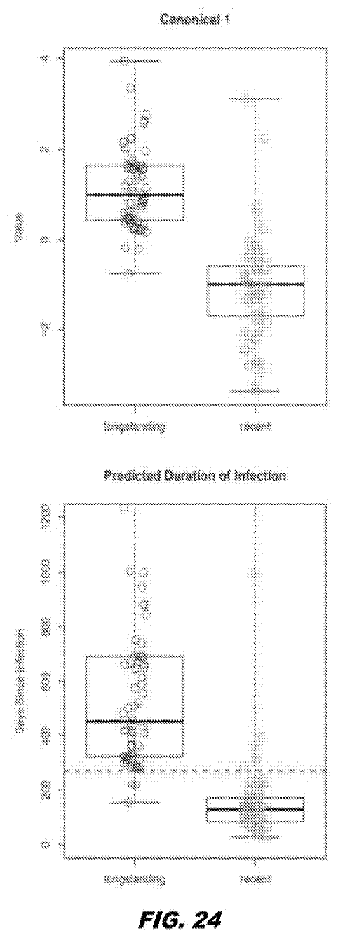

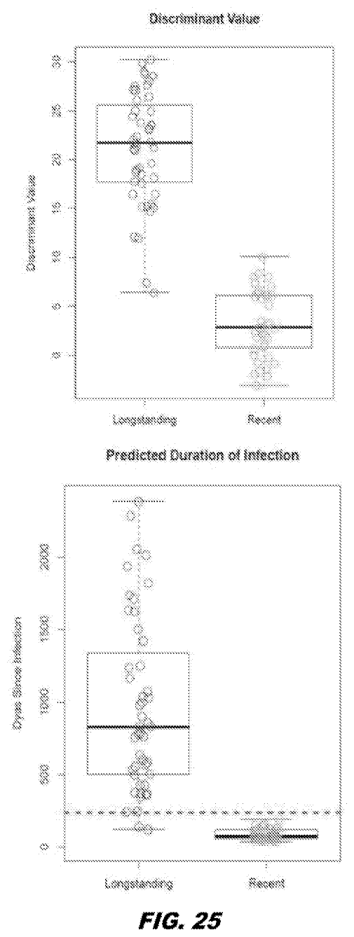

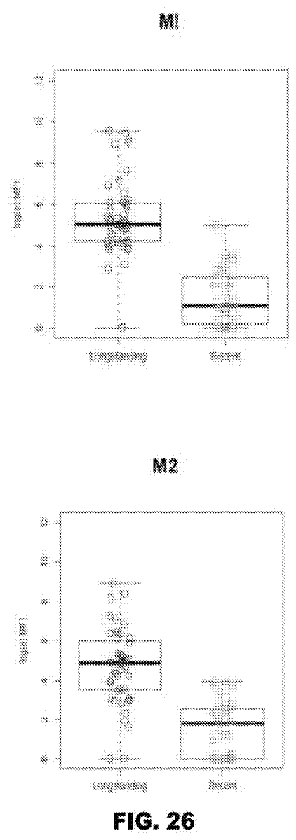

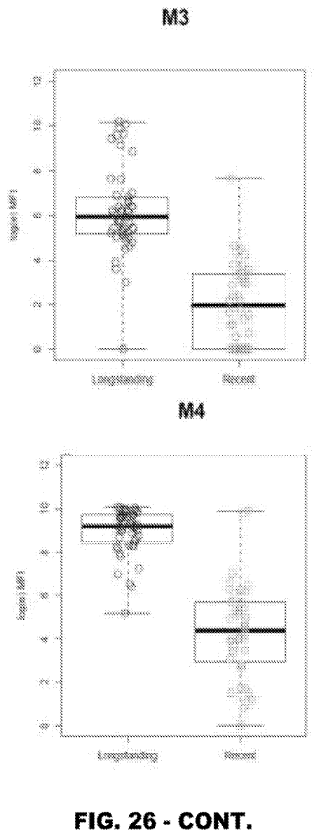

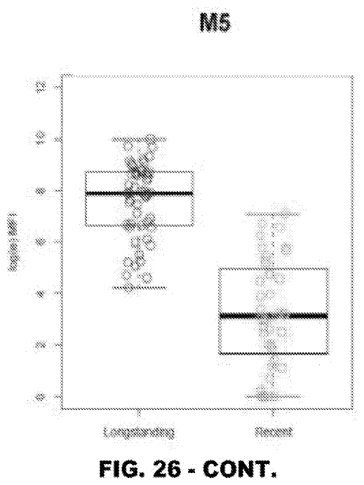

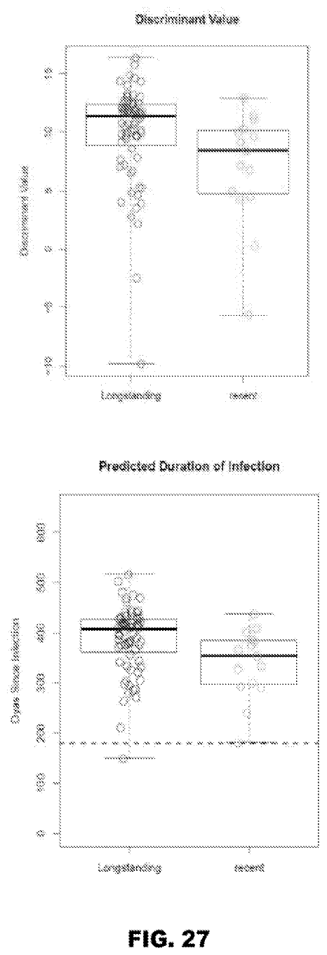

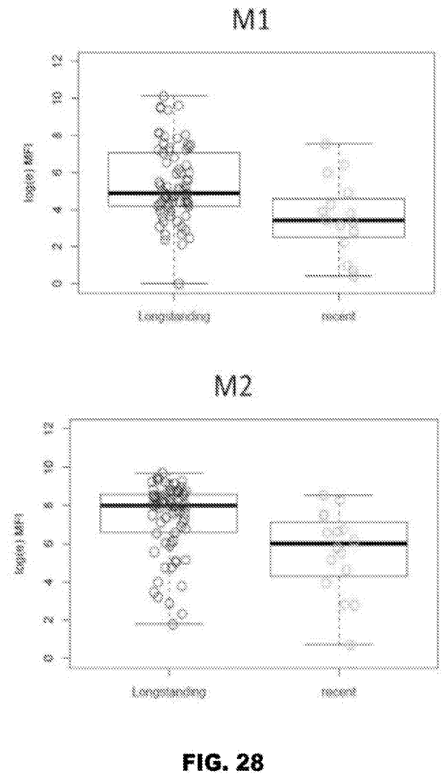

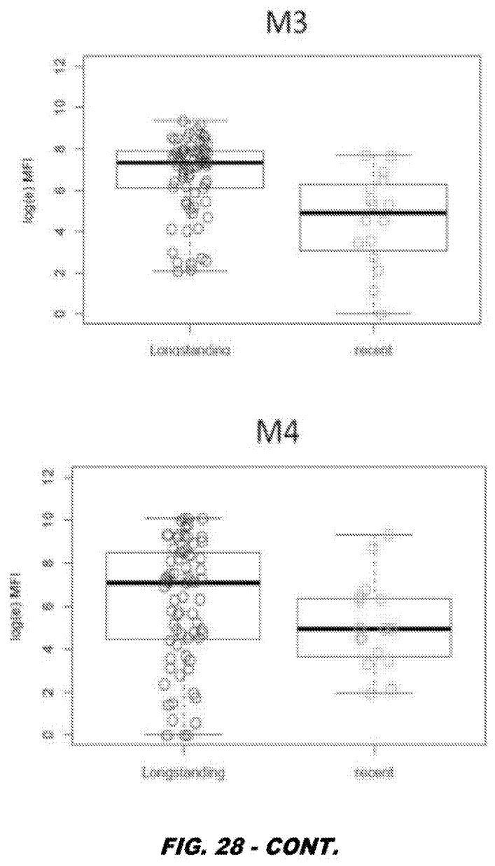

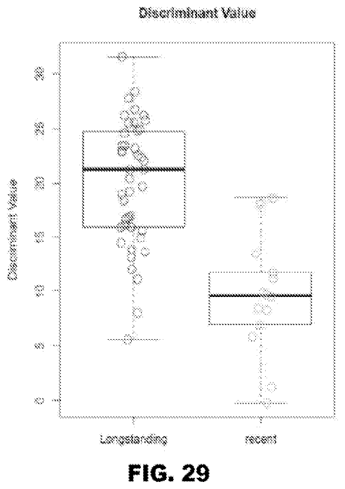

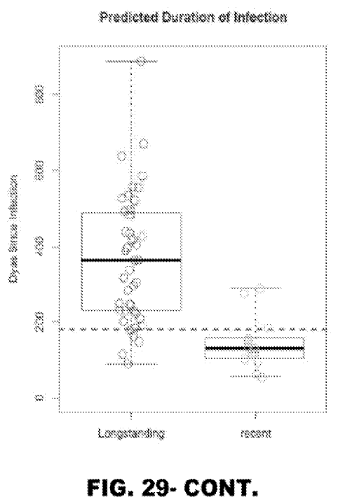

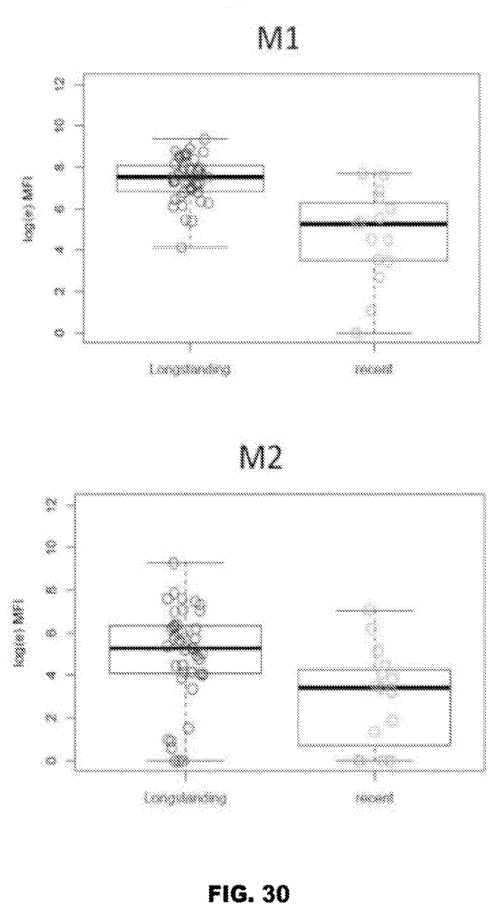

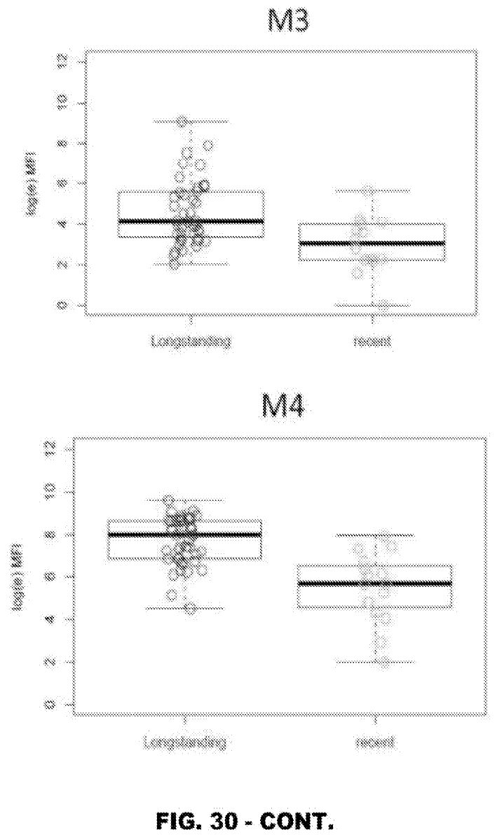

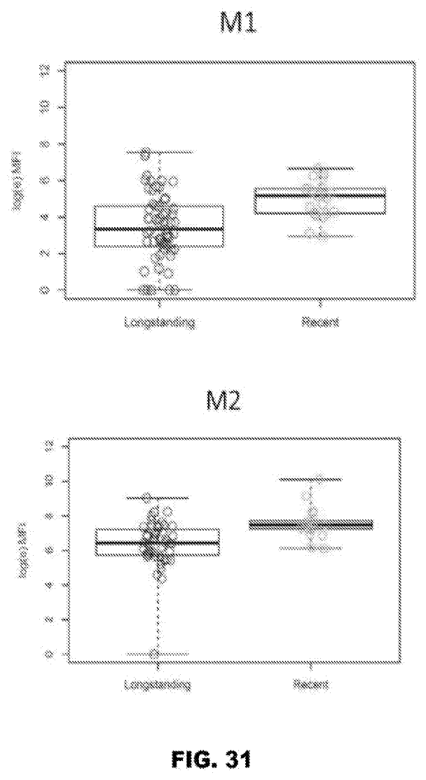

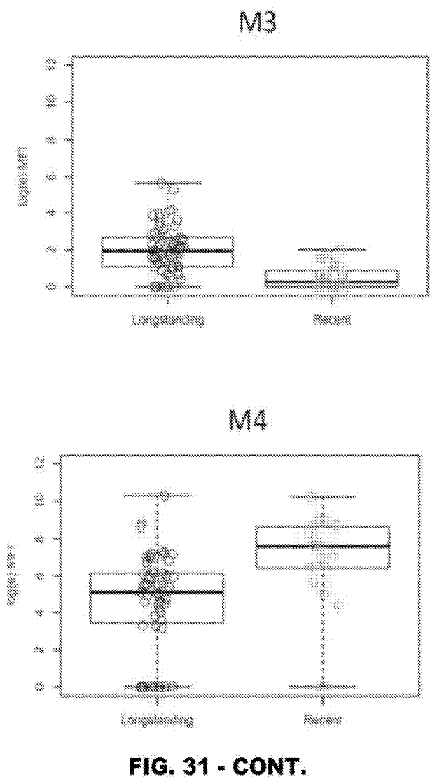

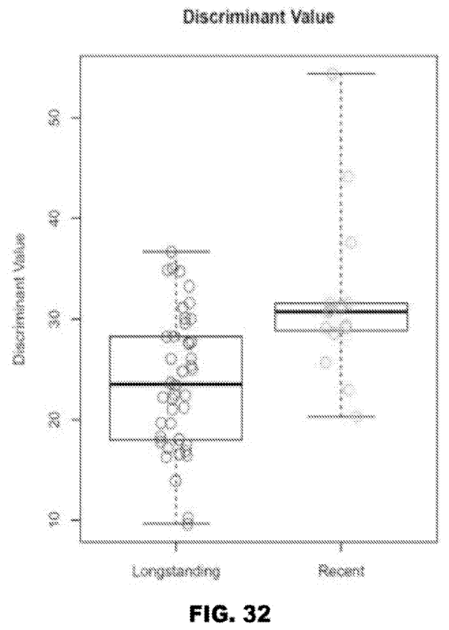

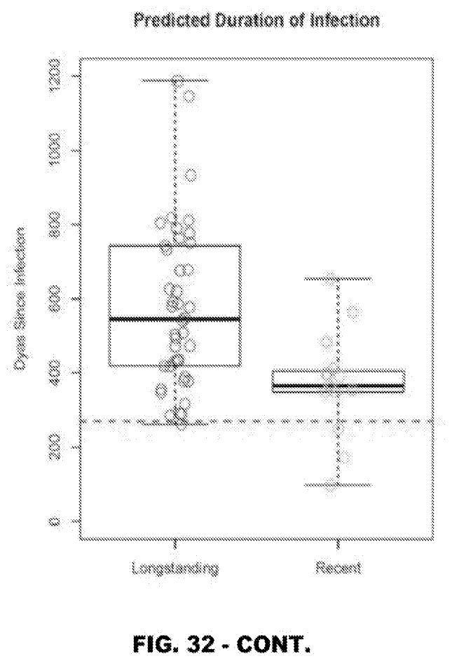

Integrated DNA serves as the template for viral RNA production. A fraction of cDNA which remains unintegrated become circularized, a reaction that is thought to be a dead end product since circular cDNA is not the substrate for the viral integrase. Circularization of viral DNA is believed to be mediated by cellular non-homologous end joining (NHEJ) pathway (FIG. 16).

Linear cDNA, the product of reverse transcription, is susceptible to a number of fates other than integration into host chromatin as proviral DNA. Autointegration may lead to the formation of truncated or internally rearranged circular forms. Although recombination may yield 1-LTR circles, host factors may also contribute their presence. Host factors, such as those involved in the non-homologous end joining pathway, participate in the formation of 2-LTR circles. Various DNA repair factors and restriction factors may also result in direct degradation of linear cDNA. Collectively, these processes help to explain patterns of unintegrated viral DNA present in infected cells. See, http://www.ncbi.nlm.nih.gov/pmc/articles/PMC4151347/ (FIG. 16)

Circular DNA formation in the nucleus in the cell nucleus is accomplished by: 1) Joining of two ends of full-length cDNA having complete LTRs to form 2-LTR circle (7, 8, 9); 2) Formation of 1-LTR circle as a result of recombination between 2-LTRs (10). 3) Circularization of stalled reverse transcription complexes that lead to 1-LTR circle. 4) Integration of the linear cDNA into itself yielding an internally arranged form (9). The amount of 2-LTR circles generated is always less than that of 1-LTR circles and independent of cell line used. The ratio of 2-LTR circles to 1-LTR circles generally range from 0.16 to 0.43 (11). Using HIV-1 based vectors, it has been shown that 2-LTR circles accumulate in cells more slowly compared to full-length cDNA and reach a maximum abundance after 24 hours, consistent with the expected precursor-product relationship. The 2-LTR circles were also found to be notably stable in the host cell nucleus (FIG. 16).

Though 2-LTR circle junctions are only a fraction of total viral cDNA in the infected cells. The amount of 2-LTR circle junctions accumulated in the nucleus is proportional to the amount of nuclear import of viral DNA since this form of viral cDNA is found only in the host nucleus. Secondly, 2-LTR circle DNA is a byproduct rather than an intermediate of the integration event (FIG. 16).

CAVL Summary.

CEPHIA panel results showed that CD4⁺ T-cell total CAVL (gag DNA) was undetected in HESN and additional data required to classify by subset ratio (collection in progress). Panel results also showed that CD4⁺ T-cell 2-LTR circle HIV DNA was undetected in HESN (n=23) and elite controllers (n=5) for all subsets, and rarely detected in naïve cells for recents, more common in longstandings. Furthermore, combining CAVL measurements for gag and 2-LTR circles into a single model may improve accuracy.

CEPHIA/CHAVI Panel Description.

Table 8 shows specimens used were HIV+ with Recent and Longstanding infections obtained from CEPHIA (N=43) and CHAVI (N=47).

TABLE 8

| Source | Classification | N |
|---|---|---|
| CEPHIA | Longstanding | 27 |
| CEPHIA | Recent (12 mos) | 16 |
| CHAVI | Longstanding | 23 |
| CHAVI | Recent (12 mos) | 24 |

CHAVI 001 (Clades B and C) classification: Cutoff for recent/longstanding=12 months; Estimated days since infection based upon Fiebig staging at Enrollment using mean cumulative duration in Cohen, et al, JID 2010.

TABLE 9
Proof of Concept Panel: Pitt Panel 2 Characteristics

| | | | | | | | | Days from |
|---|---|---|---|---|---|---|---|---|
| Classification | Total | # PID | # PID | | | | VL range | EDSC to |
| (9 months) | # PID | OFF ART | ON ART | Clade A | Clade B | Clade C | (cp/ml) | draw (range) |
| Recent | 21 | 21 | 0 | 7 | 4 | 10 | 445-6.5 × 10⁶ | −6 to 254 |
| Longstanding | 63 | 34 | 29 | 13 | 40 | 10 | <40-452,000 | 280 to >4700 |

### Example 4: Statistical Methods for Incidence Measurement

Subjects. Specimens used were HIV+ with Recent and Longstanding infections obtained from CEPHIA (N=43) and CHAVI (N=47).

TABLE 10
Subjects Used.

| Source | Classification | N |
|---|---|---|
| CEPHIA | Longstanding | 27 |
| CEPHIA | Recent | 16 |
| CHAVI | Longstanding | 23 |
| CHAVI | Recent | 24 |

CHAVI 001 (Clades B and C) classification: Cutoff for recent/longstanding=12 months; Estimated days since infection based upon Fiebig staging at Enrollment using mean cumulative duration in Cohen, et al, JID 2010.

Statistical Issue.

We have 280 measurements (run on the Luminex platform) for use as predictors of recent or longstanding—how to determine which are useful in a discriminant analysis. No easy path to finding the best set of predictors from 280 options. 7 methods for measurement selection.

Methods for Down-Selecting Measurements.

(1) Use each measurement as a continuous predictor of Classification in a logistic regression; (2) Use each measurement as a categorical predictor of Classification in a logistic regression; (3) Combined continuous and categorical logistic; (4) Rank order measurements by the mean difference between Classification; (5) Rank order measurements by the difference in positive response rate; (6) Combined mean difference and positive response rate; and (7) Rank order measurements by the median difference and absolute threshold.

Down-Selection Results.

For methods 1-5, we selected the top 20 measurements. We selected 19 measurements from method 6 and 17 measurements from method 7. The top measurements for each method were not required to be mutually exclusive.

Algorithm for Determining “Best” Measurements.

For each of the 7 methods, we ran a discriminant function analysis (DFA) for all possible sets of 2-7 measurements; we recorded the misclassification error rate for the solution for all possible sets; and we determined the lowest error rate for each method by measurement combination and output the measurements in that solution.

DFA Algorithm Results.

Methods 1, 5, and 7 produced misclassification error rates of 3%, the lowest we observed, for 5, 6, and 7 measurement sets. We chose 5 measurement sets by the principle of parsimony (although further analysis showed that 6 or more yielded only additional non-informative measurements). We gathered all 5 measurement sets from methods 1, 5, and 7 that had a misclassification error rate of 3%. There were 17 measurements that were present in any of the solutions with misclassification error rate of 3% (2 subjects misclassified). These were our “best measurements”. One subject (on ART) was always misclassified as recent. The second misclassified subject vacillated between 2 other subjects: 1 on ART and misclassified as recent; and 1 Fiebig Stage 6 and misclassified as longstanding. To select the “best” of these sets, we found the solution with the highest probability of correct classification for the misclassified subjects.

Calculation of DV.

From the best solution, output the vector of regression parameters for the measurements. We computed the discriminant value (DV) for each person based on their measurement results. Compute DV using FDA parameter estimates for the group j that subject i was classified into:

DVᵢ=β₀ⱼ+β₁ⱼM₁ᵢ+ . . . +β₅ⱼM₅ᵢ

Calculation of PDI. Using the loge of EDSC and the DV we estimate regression coefficients for EDSC: EDSCi=β₀+β₁DVᵢ+εᵢ

Using the regression coefficients above, we calculate the predicted days of infection (PDI): PDIᵢ=β₀+β₁DVᵢ

PDI is in logₑ units, so we transform to days. The MDRI is the mean of the PDI.

Best 5 Measurement Set Results.

TABLE 11
Results.

Assigned Group

| | Longstanding | Recent | Total |
|---|---|---|---|
| Longstanding | 45 | 2 | 47 |
| Recent | 0 | 39 | 39 |
| | | | 86 |

TABLE 12

| Classification | Assigned Group | | Total | N | Min | Max | Mean | SD |
|---|---|---|---|---|---|---|---|---|
| Longstanding | Longstanding | EDSC | 45 | 19 | 411.0 | 3614.0 | 1128.1 | 794.3 |
| Longstanding | Longstanding | PDI | 45 | 45 | 246.2 | 2386.8 | 1003.7 | 590.8 |
| Longstanding | Longstanding | DV | 45 | 45 | 12.0 | 30.2 | 21.8 | 5.0 |
| Longstanding | Recent | EDSC | 2 | 0 | | | | |
| Longstanding | Recent | PDI | 2 | 2 | 123.8 | 139.5 | 131.6 | 11.1 |
| Longstanding | Recent | DV | 2 | 2 | 6.5 | 7.4 | 6.9 | 0.7 |
| Recent | Recent | EDSC | 39 | 14 | 36.0 | 142.0 | 95.2 | 37.7 |
| Recent | Recent | PDI | 39 | 39 | 37.5 | 194.6 | 90.2 | 38.4 |
| Recent | Recent | DV | 39 | 39 | −3.1 | 10.1 | 3.2 | 3.4 |
| Recent | Longstanding | | 0 | | | | | |

Pitt Panel 2.

The Pitt Panel is longitudinal, which does not meet our needs; therefore, a random selection of unique subjects was selected. The 16 “best measurements” (one redundant measurement was dropped) from CEPHIA DP were run against the selected subjects. The definition of Recent and Longstanding were left to us, we defined it several ways: Strict cutoff at 6, 9, 10, 11 and 12 months; Combination of Recent <6 months and Longstanding >12 months; and Removing subjects on ART and a strict cutoff of 6 and 9 months.

Pitt Panel Subjects.

Subjects were selected using a simple random sample without replacement from the different points of measurement, 1, 2 and 3. Note: Total=Total number of subjects, N=subjects with EDSC measurement

TABLE 13

| Months | Classification | Total | N | Mean | SD | Min | Max |
|---|---|---|---|---|---|---|---|
| 6 | Longstanding | 73 | 53 | 1016.6 | 1310.2 | 187 | 4737.5 |
| 6 | Recent | 16 | 15 | 91.6 | 59.8 | −6 | 173 |
| 9 | Longstanding | 63 | 47 | 1117.8 | 1359.4 | 280 | 4737.5 |
| 9 | Recent | 21 | 21 | 129.2 | 80.5 | −6 | 254 |
| 10 | Longstanding | 60 | 45 | 1154.8 | 1378.1 | 307.5 | 4737.5 |
| 10 | Recent | 23 | 23 | 142.9 | 89.2 | −6 | 294 |
| 11 | Longstanding | 50 | 37 | 1334.9 | 1460.7 | 335.5 | 4737.5 |
| 11 | Recent | 31 | 31 | 189.1 | 110.4 | −6 | 335 |
| 12 | Longstanding | 46 | 33 | 1454.1 | 1504.9 | 373 | 4737.5 |
| 12 | Recent | 35 | 35 | 207.6 | 116.2 | −6 | 361 |
| Rec < 06 Long > 12 | Longstanding | 46 | 33 | 1454.1 | 1504.9 | 373 | 4737.5 |
| Rec < 06 Long > 12 | Recent | 16 | 15 | 91.6 | 59.8 | −6 | 173 |
| No ART 06 | Longstanding | 43 | 36 | 407.5 | 151.4 | 187 | 777 |
| No ART 06 | Recent | 15 | 14 | 97.9 | 56.8 | −6 | 173 |
| No ART 09 | Longstanding | 33 | 30 | 444.3 | 138.0 | 280 | 777 |
| No ART 09 | Recent | 20 | 20 | 135.5 | 77.1 | −6 | 254 |

DFA Algorithm.

From the 16 measurements, run DFA on all possible measurement sets of size k. Output the misclassification error rate and the measurements used for all possible solutions. Find the lowest possible error rate.

Pitt Panel 2 Study Design.

Definition of Recent/Longstanding: 7 different; Number of measurements: 4,5,6,7,8; Design (22 Classification x measurement conditions): (a) Classification=6 months: 4-8 measurement sets, (b) Classification=6 months NO ART: 4-6 measurement sets, (c) Classification=6 Rec>12 Long: 4-6 measurement sets, (d) Classification=9 months: 4-8 measurement sets, (e) Classification=9 months NO ART: 4-6 measurement sets, (0 Classification=10 months: 5 measurement sets, (g) Classification=11 months: 5 measurement sets, and (h) Classification=12 months: 5 measurement sets.

DFA Algorithm Results.

TABLE 14
Results for each condition listed below.

| Classification | Set Size | N | Min | Max | Mean | SD |
|---|---|---|---|---|---|---|
| No ART: Recents < 06 Months | 4 Measurements | 1820 | 0.09 | 0.32 | 0.18 | 0.04 |
| | 5 Measurements | 4368 | 0.09 | 0.32 | 0.18 | 0.04 |
| | 6 Measurements | 8008 | 0.09 | 0.32 | 0.18 | 0.04 |
| No ART: Recents < 09 Months | 4 Measurements | 1820 | 0.11 | 0.38 | 0.23 | 0.04 |
| | 5 Measurements | 4368 | 0.11 | 0.38 | 0.23 | 0.04 |
| | 6 Measurements | 8008 | 0.11 | 0.40 | 0.23 | 0.04 |
| Rec < 06 Long > 12 | 4 Measurements | 1820 | 0.15 | 0.38 | 0.26 | 0.04 |
| | 5 Measurements | 4368 | 0.12 | 0.38 | 0.25 | 0.04 |
| | 6 Measurements | 8008 | 0.12 | 0.37 | 0.25 | 0.04 |
| Recents < 06 Months | 4 Measurements | 1820 | 0.09 | 0.23 | 0.18 | 0.02 |
| | 5 Measurements | 4368 | 0.09 | 0.24 | 0.18 | 0.02 |
| | 6 Measurements | 8008 | 0.09 | 0.24 | 0.17 | 0.02 |
| | 7 Measurements | 11440 | 0.09 | 0.24 | 0.17 | 0.03 |
| | 8 Measurements | 12870 | 0.09 | 0.26 | 0.17 | 0.03 |
| Recents < 09 Months | 4 Measurements | 1820 | 0.17 | 0.33 | 0.25 | 0.03 |
| | 5 Measurements | 4368 | 0.17 | 0.34 | 0.25 | 0.03 |
| | 6 Measurements | 8008 | 0.16 | 0.35 | 0.24 | 0.03 |
| | 7 Measurements | 11440 | 0.14 | 0.35 | 0.24 | 0.03 |
| | 8 Measurements | 12870 | 0.14 | 0.34 | 0.24 | 0.03 |
| Recents < 10 Months | 5 Measurements | 4368 | 0.18 | 0.38 | 0.27 | 0.03 |
| Recents < 11 Months | 5 Measurements | 4368 | 0.25 | 0.53 | 0.38 | 0.05 |
| Recents < 12 Months | 5 Measurements | 4368 | 0.23 | 0.63 | 0.40 | 0.06 |

TABLE 15
Measurements in the best solutions

| | Total number of solutions ~9% | | | | | |
|---|---|---|---|---|---|---|
| | 1 NOART | 1 | 5 | 9 | 13 | 13 |
| measurement | N_4Ags | N_4Ags | N_5Ags | N_6Ags | N_7Ags | N_8Ags |
| IgG_CIT_A1_con_env03140CF | | 1 | 5 | 9 | 13 | 13 |
| IgG_CIT_B_con_env03140CF | 1 | 1 | 5 | 9 | 13 | 13 |
| IgG_PBS_Bio_V3_A | | 1 | 5 | 8 | 13 | 13 |
| IgG_PBS_Bio_V3_C | | | | 3 | 10 | 11 |
| IgG4_PBS_1086Trimer | | 1 | 5 | 6 | 5 | 8 |
| IgG_CIT_Bio_V3_B | 1 | | 1 | 4 | 4 | 7 |
| IgG_PBS_Bio_V3_B | | | 1 | 5 | 7 | 7 |
| IgG4_PBS_p66 | | | | 2 | 6 | 7 |
| IgA_CIT_1086Trimer | | | | 1 | 3 | 6 |
| IgG4_PBS_C_con_env03140CF_avi | 1 | | 1 | 3 | 4 | 6 |
| IgG_CIT_gp41 | 1 | | | 1 | 4 | 5 |
| IgG_PBS_p31 | | | 1 | 2 | 5 | 4 |
| IgG4_PBS_B_con_env03140CF | | | 1 | 1 | 4 | 3 |
| IgG4_PBS_A1_con_env03140CF | | | | | | 1 |
| IgA_CIT_A1_con_env03140CF | | | | | | |
| IgG_PBS_A244gp120gDneg_293F_mon | | | | | | |

Recent <6Mo, Best 4 Results.

TABLE 16

Assigned Group

| | Longstanding | Recent | Total |
|---|---|---|---|
| Longstanding | 72 | 0 | 72 |
| Recent | 8 | 7 | 15 |
| | | | 87 |

TABLE 17

| Classification | Assigned Group | | Total | N | Min | Max | Mean | SD |
|---|---|---|---|---|---|---|---|---|
| Longstanding | Longstanding | EDSC | 72 | 52 | 187.0 | 4737.5 | 969.4 | 1276.8 |
| Longstanding | Longstanding | PDI | 72 | 72 | 149.4 | 515.8 | 387.7 | 65.6 |
| Longstanding | Longstanding | DV | 72 | 72 | -9.8 | 16.4 | 10.0 | 4.2 |
| Longstanding | Recent | | 0 | 0 | | | | |
| Recent | Longstanding | EDSC | 8 | 8 | 48.0 | 167.5 | 114.3 | 40.5 |
| Recent | Longstanding | PDI | 8 | 8 | 300.8 | 438.2 | 377.4 | 43.5 |
| Recent | Longstanding | DV | 8 | 8 | 5.0 | 12.9 | 9.6 | 2.5 |
| Recent | Recent | EDSC | 7 | 7 | -6.0 | 173.0 | 65.7 | 70.5 |
| Recent | Recent | PDI | 7 | 7 | 182.3 | 376.2 | 295.3 | 66.9 |
| Recent | Recent | DV | 7 | 7 | -5.6 | 9.7 | 4.1 | 5.3 |

Recent <6Mo, No ART, Best 4 Ag Results.

TABLE 18

Assigned Group

| | Longstanding | Recent | Total |
|---|---|---|---|
| Longstanding | 42 | 1 | 43 |
| Recent | 4 | 10 | 14 |
| | | | 57 |

TABLE 19

| Classification | Assigned Group | | Total | N | Min | Max | Mean | SD |
|---|---|---|---|---|---|---|---|---|
| Longstanding | Longstanding | EDSC | 42 | 36 | 187.0 | 777.0 | 407.5 | 151.4 |
| Longstanding | Longstanding | PDI | 42 | 42 | 114.3 | 886.8 | 371.6 | 167.5 |
| Longstanding | Longstanding | DV | 42 | 42 | 8.1 | 31.5 | 20.4 | 5.4 |
| Longstanding | Recent | EDSC | 1 | 0 | | | | |
| Longstanding | Recent | PDI | 1 | 1 | 91.8 | 91.8 | 91.8 | |
| Longstanding | Recent | DV | 1 | 1 | 5.5 | 5.5 | 5.5 | |
| Recent | Longstanding | EDSC | 4 | 4 | 98.0 | 167.5 | 134.5 | 28.8 |
| Recent | Longstanding | PDI | 4 | 4 | 150.2 | 287.6 | 224.3 | 67.6 |
| Recent | Longstanding | DV | 4 | 4 | 11.2 | 18.6 | 15.4 | 3.6 |
| Recent | Recent | EDSC | 10 | 10 | −6.0 | 173.0 | 83.2 | 59.5 |
| Recent | Recent | PDI | 10 | 10 | 55.6 | 156.8 | 110.5 | 31.9 |
| Recent | Recent | DV | 10 | 10 | −0.2 | 11.7 | 7.1 | 3.9 |

Why Didn't CEPHIA DP Set Work for PITT 2?

Statistical theory tells us that two random samples from the same population will have similar statistical properties. Are they from different populations? Could be a differential bias in EDSC estimation. Were the cohorts calculated the same? Is the “margin of error” the same?

Pitt Panel 2 New Measurements.

7 Antibody Isotypes/Subclasses examined. Analytes: 103 envs, peptides or proteins. Measurements: 505 unique combinations of Isotype and Analyte

Methods for Down-Selection for Pitt Panel 2.

(1) Use each measurement as a continuous predictor of Classification in a logistic regression; (2) Use each measurement as a categorical predictor of Classification in a logistic regression; (3) Rank order measurements by the absolute value of the correlation with EDSC; (4) Rank order measurements by the mean difference between Classification; (5) Rank order measurements by the difference in positive response rate; (6) Rank order measurements by the median difference and absolute threshold; and (7) Combine the order score for methods 1-6.

Down-Selection Results for Pitt Panel 2.

For methods 1-5, we selected the top 20 measurements. We selected 15 measurements from method 6. We selected 20 measurements from method 7. The top measurements for each method were not required to be mutually exclusive.

Algorithm for Determining “Best” Measurements.

For each of the 7 methods, we ran a discriminant function analysis (DFA) for all possible sets of 3-6 measurements; we recorded the misclassification error rate for the solution for all possible sets; and we determined the lowest error rate for each method by measurement combination and output the measurements in that solution.

Stats on Algorithm. Number of possible measurement combinations for N measurements choosing K sized sets:

$$\frac{N !}{K ! ( N - M ) !}$$

TABLE 20

| N | K | Per | Total |
|---|---|---|---|
| 20 | 3 | 1140 | 7980 |
| 20 | 4 | 4845 | 33915 |
| 20 | 5 | 15504 | 108528 |
| 20 | 6 | 38760 | 271320 |
| | | | 421743 |

DFA Algorithm Results.

Method 1 produced misclassification error rates of ˜3.2%, the lowest we observed. We observed this rate for 4, 5 and 6 measurement sets. We chose 4 measurement sets by the principle of parsimony.

TABLE 21

| Measurements | Method 1 | Method 2 | Method 3 | Method 4 | Method 5 | Method 6 | Method 7 |
|---|---|---|---|---|---|---|---|
| 3 | 4.8% | 11.5% | 10.8% | 8.3% | 9.2% | 11.7% | 7.0% |
| 4 | 3.2% | 10.4% | 9.9% | 8.3% | 8.0% | 13.0% | 5.6% |
| 5 | 3.2% | 8.3% | 8.8% | 7.0% | 5.6% | 13.0% | 5.6% |

We gathered the 4 measurement sets (N=3) from method 1 that had a misclassification rate of ˜3.2%. There were 6 measurements that were present in any of the solutions with misclassification rate of ˜3.2% (2 subjects misclassified).

TABLE 22

| Specimen ID | Classification09 | Assigned Group | N |
|---|---|---|---|
| 5874-01 | Longstanding | Recent | 2 |
| 0702-01 | Recent | Longstanding | 1 |
| 1976-01 | Recent | Longstanding | 2 |
| 7328-01 | Recent | Longstanding | 1 |

One solution did not misclassify any Longstanding as Recent (obs=841). This was our “best solution”. Missing data issue: N=21 longstanding and N=6 recent due to assay sensitivity. Assay being tuned.

Best 4 Measurement Set Results.

TABLE 23

Assigned Group

| | Longstanding | Recent | Total |
|---|---|---|---|
| Longstanding | 42 | 0 | 42 |
| Recent | 2 | 13 | 15 |
| | | | 57 |

TABLE 24

| Classification | Assigned Group | | Total | N | Min | Max | Mean | SD |
|---|---|---|---|---|---|---|---|---|
| Longstanding | Longstanding | EDSC | 42 | 29 | 280.0 | 4737.5 | 1442.0 | 1623.4 |
| Longstanding | Longstanding | PDI | 42 | 42 | 261.7 | 1188.2 | 575.9 | 221.0 |
| Longstanding | Longstanding | DV | 42 | 42 | 9.6 | 36.7 | 23.8 | 6.8 |
| Longstanding | Recent | | 0 | 0 | | | | |
| Recent | Longstanding | EDSC | 2 | 2 | 167.5 | 187.0 | 177.3 | 13.8 |
| Recent | Longstanding | PDI | 2 | 2 | 398.9 | 564.0 | 481.5 | 116.7 |
| Recent | Longstanding | DV | 2 | 2 | 23.0 | 29.1 | 26.0 | 4.4 |
| Recent | Recent | EDSC | 13 | 13 | −6.0 | 251.5 | 114.7 | 71.3 |
| Recent | Recent | PDI | 13 | 13 | 97.2 | 653.9 | 356.6 | 136.6 |
| Recent | Recent | DV | 13 | 13 | 20.3 | 54.4 | 32.7 | 8.6 |

### Example 5: Novel Antibody Biomarkers for Detection of Incident HIV-1 Infection

Introduction. Accurate cross-sectional estimates of recent HIV-1 infection are key in determining success and/or uptake of HIV-1 prevention methods (PrEP, PEP, behavioral interventions), and determine required sample size for HIV-1 vaccine trials. However, currently approved cross-sectional surveillance methods have limited performance, particularly in “difficult to classify” populations including HIV+ individuals on ART, elite controllers/long-term non-progressors, and Subtypes A and D infection. A major limitation of current assays is a high false-recent rate (FRR), where chronically infected HIV+ individuals score as recently infected. Thus, evaluation of novel HIV-1 biomarkers to characterize recent vs. longstanding HIV infection with a low FRR is needed.

Study Objectives.

(1) To determine the presence, magnitude and avidity of IgG, IgA, IgM binding antibody responses to a panel of HIV-1 specific antigens in recent (<9 months) vs longstanding infection; (2) To downselect a panel of 4-7 antibody/antigen pairs that achieves the lowest FRR in a globally relevant cross-sectional panel of specimens; and (3) To develop a model for determination of incident infection in a test population.

Samples.

Plasma samples were obtained from the following cohorts and used with permission from the respective cohorts and Institutional IRB approvals.

CEPHIA Development Panel 2 (CEPHIA):

Clade B, ARV naïve, 5 elite controller specimens, 35 longstanding infection, 16 recent (<12 months), 24 HIV negative specimens.

Pitt Panel 2 (CEPHIA):

Longitudinal specimens, 30 ARV treated, 130 longstanding, 62 recent (<9 months), Clades A, B, C.

RV217 (USMHRP):

Longitudinal specimens obtained during acute HIV-1 infection. 15 samples from recent (<9 months) infection; all ARV naïve, Clades A and A/E.

CHAVI 001 (CHAVI):

26 Recent (<9 months) and 24 longstanding; Clades B and C infection.

Methods.

BAMA.

We utilized the Binding Antibody Multiplex Assay (BAMA)¹⁻⁴ to assess HIV-1 specific binding antibody responses to a panel of Env and non-Env antigens. dIgA responses were detected using the pIgR reagent (David Anderson, Burnet Institute). Avidity measurements were determined as the Mean Fluorescence Intensity (MFI) of samples treated with Na-Citrate, pH 3.0.

Methods for Antigen/Antibody Downselection Using Discriminant Function Analysis.

(1) Use each measurement as a continuous predictor of Classification in a logistic regression. (2) Use each measurement as a categorical predictor of Classification in a logistic regression. (3) Rank order measurements by the absolute value of the correlation with EDSC. (4) Rank order measurements by the mean difference between Classification. (5) Rank order measurements by the difference in positive response rate. (6) Combine the order score for methods 1-5. (7) Use the pairwise difference as a continuous predictor of Classification in a logistic regression.

Conclusions.

(a) We have identified multiple subsets of HIV-1 specific antigen/antibody pairs that are excellent candidates for determination of recent (<9 months) vs longstanding infection; (b) Additionally, a subset of 4 direct binding and antibody avidity measurements are able to distinguish recent (<9 months) vs longstanding infection in a globally relevant panel with a low (4.5% FRR). This FRR includes samples from patients treated with ARV; and (c) These antibody measurements are excellent candidates to advance for further development of a novel HIV-1 incidence algorithm.

Proof-of-Concept Phase—a Panel of 4 Antigens Predicts Recent (<9 Months) Infection with 0% FRR and 3.5% Total Error Rate.

TABLE 25
Binding antibody responses were measured to a panel of 281
antigen/antibody pairs and analyzed using discriminant function
analysis. FRR = false resent rate, Error rate =
total misclassification rate

| | Longstanding | Recent by new | |
|---|---|---|---|
| | by new assay | assay | Total |
| Nominally | 42 | 0 | 42 |
| Longstanding* | | | |
| Nominally Recent | 2 | 13 | 15 |
| Total | 44 | 13 | 57 |

Top 3 solutions**: FRR: 0%, Error Rate: 3.5% FRR: 1.6%, Error Rate: 3.2% FRR: 1.6% Error Rate: 3.2%
Panel Characteristics: Pitt Panel 2 (Clades A, B, C: 23 ART)

The contents of all references and publications are hereby incorporated by reference in their entirety.

The entire content of US Publication 2013/0217002 is hereby incorporated by reference.

### Example 6: Multiparameter Measurements of HIV-1 Specific Antibody Subclass and Isotype Distinguish Recent and Longstanding HIV-1 Infection

Abstract

Background:

Current HIV-1 incidence assays are limited by inability to distinguish recent vs. longstanding HIV infection in some “difficult to classify” populations (e.g. ART treatment, elite controllers). HIV-1 incidence estimation is important to determine success of HIV-1 prevention strategies. The early HIV-1 antibody (Ab) response is remarkably dynamic (e.g. staged response to HIV antigens, differential kinetics of Ab isotypes/subclasses) and can be harnessed to identify novel biomarkers for improved incidence assays.

Methods:

To develop an improved predictor of recent HIV infection, we conducted retrospective analyses on antibodies circulating in plasma from recent and longstanding infections from three different acute infection cohorts (i.e. CHAVI 001, RV217, and CEPHIA) comprising subtypes A, B and C. We examined binding and avidity measurements comprised of Env and non-Env Ags and multiple subclasses and isotypes: IgG, IgA, IgG3, IgG4, dIgA, IgM) on plasma from recent and longstanding infections from multiple clades including those on anti-retroviral therapy (ART). The top 15-20 measurements from each assessment were evaluated by machine learning with discriminant function analysis (DFA). All possible sets of 3-5 antibody/antigen combinations were evaluated by DFA to identify solutions with the lowest misclassification rate.

Results:

The solution with the lowest total misclassification rate comprised 4 Env binding and avidity measurements including: IgG3 Clade C gp140, IgG4 avidity T/F Clade C gp140, IgG4 avidity Clade B gp140, and IgG avidity gp41 ID. Difference score: IgG Citrate gp41 ID epitope—IgG PBS gp41 ID epitope. Using a recency cutoff of 9 months, these measurements misclassified longstanding infection at a rate of 4.5%, including ART treatment. False Recent Rate (FRR) using the field standard of T=2 yrs was 0%.

Conclusions:

We have identified multiple subsets of HIV-1 specific antibody/antigen pairs that distinguish recent (<9 months) vs. longstanding infection in a globally relevant panel with a 0-4.5% false-recent misclassification rate, that notably included ARV treated patients. Commercially available incidence assays misclassify between 12.9%-76.1% of elite/ART treated patients. Thus, the multiparameter measurements of HIV-1 recency reported here are excellent candidates to advance for further development of a novel HIV-1 incidence algorithm.

### Introduction

Accurate estimates of HIV-1 incidence (i.e. the number of new HIV infections in a population in a defined period of time) are critical for planning and evaluating the success of HIV-1 prevention strategies[C1, C2]. Recent advances in novel preventative measures, including the one partially efficacious vaccine regimen[C3], treatment-as-prevention[C4] and Pre-Exposure Prophylaxis (PReP)[5], have changed the landscape of effective preventive measures. These and next generation prevention strategies building on these successes need more accurate HIV incidence measures to fully understand their impact on the global epidemic. Currently available cross-sectional HIV-1 incidence assays have limited utility in difficult to classify populations (ART treatment, elite controllers and Subtypes A, D infection), due to high False Recent Rates (FRR) [C6]. Development of HIV-1 incidence assays using novel biomarkers with improved FRR is critical to the assessment of HIV-1 treatment and prevention efforts, the design of HIV-1 vaccine trials, and monitoring the epidemic in pursuit of an AIDS free generation. Improvements in HIV-1 incidence must meet several key criteria for assay performance [C2, C6]. These include the Mean Duration of Recent Infection (MDRI), or length of time the biomarker accurately predicts recent infection, and FRR, the proportion of longstanding subjects infected greater than time T (e.g. 2 years) that appear to be recently infected in incidence assays [6]. The Incidence Assay Critical Path Working Group [C2] recommends an ideal MDRI of between 6-12 months post infection with a FRR <2%.

The inventors, and others, have previously characterized the sequential progression of HIV-1 antibody responses in acute through chronic infection and found that HIV-1 specific antibody isotypes and subclasses are extraordinarily dynamic during the early phase post acquisition and thus may be suitable for discriminating recent from longstanding infection [C7-C11]. These include early markers of HIV infection such as gp41 and Gag IgM, IgG and IgA antibodies, which are among the earliest antibody specificities to arise post-infection [C7, C8]. IgG antibodies to the HIV envelope are elicited sequentially, with a delay in anti-gp120 antibodies [C8]. The earliest epitope specific responses to gp41 appear first to the immunodominant region (ID) and in the V3 region for gp120, whereas CD4bs and non-neutralizing antibodies to the MPER arise later in infection [C7, C8]. Maturation of antibody responses is accompanied by an increase in antibody avidity for specific HIV-1 antigens, and can include an increase in breadth of recognition of multiple HIV-1 subtypes during the transition from recent to chronic infection. Notably, some antibody responses exhibit a declining kinetics during the early phase of infection. IgG3 antibody responses to p55 Gag, gp41 Env, and p66 RT decline in acute infection, in contrast to the same antigen specific IgG1 responses [C9, C10] Env IgA also demonstrates a marked decline both systemically and mucosally during recent infection [C7]. Thus, the inventors hypothesized that a multiparameter approach that includes antibody isotypes, and subclasses, presence or absence of certain epitope specificities, antibody avidity and recognition of unique HIV-1 envelope antigens would more accurately distinguish recent from longstanding infection.

Our approach builds upon this previous work profiling antibody kinetics from acute to chronic infection using multiplex binding measurements [C7-C9, C11, C12]. Here, the inventors determined epitope specificities and antibody isotypes and subclasses displaying distinct kinetic profiles through the course of HIV-1 infection for multiple HIV-1 subtypes. Multiparameter measurements were evaluated by machine learning with discriminant function analysis (DFA) to identify novel combinations of naturally occurring antibody biomarkers for cross-sectional HIV-1 incidence testing.

Methods.

Patient Cohorts

Plasma/serum from the HIV-1 acute and chronic cohorts: Center for HIV/AIDS Vaccine Immunology (CHAVI) (CHAVI 001, CHAVI 008), United States Military HIV Research Program (USMHRP) (RV217), and the Consortium for the Evaluation and Performance of HIV-1 Incidence Assays (CEPHIA). Retrospective analysis was performed with approval from the Duke Medicine Institutional Review Board (IRB) for Clinical Investigations (Protocol: Pro00039677). Samples were classified as “recent” infection if time since Estimated Date of Seroconversion (EDSC) or the maximum time since first HIV-1 positive test was <270 days. If EDSC was unknown, then time since infection was determined based upon Fiebig staging at enrollment using the maximum cumulative duration of infection as described by Cohen, et al. [C13]. Samples were classified as longstanding infection if time since first HIV-1 positive test was >270 days or if participants had been enrolled in an HIV-1 positive cohort for >270 days.

Binding Antibody Multiplex Assay (BAMA)

the inventors profiled HIV-1 specific binding antibody responses in participant serum or plasma as described previously [C7-C9]. The inventors evaluated antibody binding to a unique and broad panel of HIV-1 antigens that included gp120, gp160, and V1-V2 antigens of multiple subtypes. This panel includes a downselected set of envelope antigens for assessing cross-clade breadth of binding responses (e.g. 16 gp120, 16 gp140, and 16 gp70-V1V2 Env antigens) (Yates, deCamp, Tomaras et al 2016., in preparation). Additional antigens included multiple Clade specific and consensus linear peptides for the gp41 immunodominant region, V1, V2 V3, C1 and C5 epitopes, p31 integrase (Jena Bioscience), p66 reverse transcriptase (RT) (Protein Sciences), p24 Gag (BD Biosciences). Serum or plasma were diluted in BAMA assay diluent (1% milk-blotto, 5% normal goat serum, 0.05% Tween-20) and incubated antigen coupled microspheres. Samples were incubated with either anti-human IgG (Southern Biotech), anti-human IgA (Jackson Immunoresearch), anti-human IgG3 (Calbiochem), anti-human IgG4 (BD Pharmingen), or anti-human IgM (Southern Biotech) followed by Streptavidin-PE (BD Biosciences) and detection on either a Bioplex 200 (BioRad, Hercules, Calif.) or Luminex FM3D machine (Luminex Corp, Austin, Tex.). All samples were depleted of IgG using a MultiTrap system (GE Biosciences) per manufacturer's instruction, prior to assessment of IgA or IgM specific antibodies. Dimeric IgA was detected using a recombinant poly-Ig receptor (pIgR). Samples were incubated with pIgR overnight in assay diluent (1% BSA, 0.05% Tween-20), followed by incubation with microsphere beads covalently coupled to the antigen of interest. pIgR was detected using anti-human SC detection followed by anti-mouse IgG-PE (Southern Biotech). Controls for IgG assays included titrated HIVIG (NIH AIDS Reagent Program), 7B2 IgG [C14], CH58 IgG [C15], and CH22 IgG mAb [C16]. IgA specific controls included: titrated 7B2 monomeric IgA (Haynes). Controls for the pIgR assay included: 7B2 monomeric IgA, 7B2 SIgA, 7B2 dIgA ((Liao, Haynes et al., Submitted), and purified Secretory component coupled beads. Controls for IgM assays included titrated 2F5 and CLL246 (gp41 specific) IgM. Controls for IgG3 and IgG4 assays included a titrated purified human IgG3 or Ig4 standard curve, and purified IgG3 or IgG4 coupled beads. Negative controls in each assay included Normal Human Serum (NHS, Sigma Aldrich) and Blank beads. Each experiment was performed using good clinical laboratory practice (GCLP)-compliant conditions, including tracking of positive controls by Levey-Jennings charts.

Antibody Avidity

Assessment of antibody avidity was determined by BAMA with the following modifications. After formation of antigen antibody immune complexes, a 15 minute dissociation step (Na-Citrate, pH 3.0, Teknova) [C17] was included prior to addition of secondary detection antibody. Retained binding magnitude (MFI) in the presence of dissociation buffer was used as a measurement of avidity in the statistical models.

HIV-1 Limiting Antigen (LAg)-Avidity EIA

RV217 and CHAVI 001 specimens were tested at the Immunology Quality Assessment Center (IQA, Duke University, Durham, N.C.), and the CEPHIA specimens were tested at Blood Systems Research Institute (BSRI, San Francisco, Calif., USA). All samples were evaluated for recency using the SEDIA HIV-1 Limiting Antigen (LAg)-Avidity EIA per manufacturer's instructions (SEDIA Biosciences Corporation, Portland, Oreg., USA).

MLV Neutralization for ART Detection

The inventors assessed samples (plasma, CEPHIA Development Panel 2; urine, HRBS panel) for MLV neutralization in a TZM-bl neutralization assay as previously described [18]. The MLV Env pseudotype backbone contains the targets for ART (e.g., RT), and thus detectable MLV neutralization titers indicate the potential presence of ART in the sample.

Statistical Methods/Discriminant Function Analysis

To down select from 505 antigen/antibody measurements the inventors used six criteria to rank order the measurements and then selected the top 20 measurements according to each criteria. Twenty was an arbitrary number selected to make the final list manageable while minimizing the risk of excluding a measurement with good discriminatory power. The six criteria were: (1) use the loge MFI as a continuous predictor of HIV status (recent vs longstanding) and then rank order the measurements by the Wald χ2 for each parameter; (2) use the MFI to make a categorical variable for positive/negative response based on the measurement threshold and use that as a categorical predictor predictor of HIV status and then rank order the measurements by the Wald χ2 for that parameter; (3) rank order the measurements by the absolute value of Pearson correlation coefficient with EDSC (estimated days to sero-conversion); (4) rank order the measurements by the mean difference between HIV status groups; (5) rank order measurements by the difference in positive response rate between HIV status groups; (6) compute the pairwise difference between all pairs of measures for each patient to use a continuous predictor of HIV status group and rank order by Wald χ2 for that parameter. Once the 120 measures were collected for the six criteria these lists were combined and there were 108 unique measurements.

To determine a set of measures that had the optimum predictive power the inventors applied a discriminant function analysis model (Fisher, 1936; Tabachnick and Fidell, 1996) to all possible 3, 4, 5 and 6 measurement sets from the six down selected lists of measures. The inventors recorded the misclassification error rate (recent patients categorized as longstanding and longstanding patients categorized as recent) for each possible unique combination of the 20 measurements for each down selected set. After four measurement sets, The inventors reached a lower bound total error rate of 6.6%. By the principle of parsimony, The inventors determined that the four measurement solution was the optimal solution at the same total error rate as solutions with more measurements.

Using the discriminant function analysis model to determine the final measurement set has the added attraction of leaving us with an algorithm that can be used to prospectively categorize patients as recent or longstanding based upon assay results on the four measurements in the best set. Classification coefficients for each measure can be extracted from the model and new measures fed through the classification equation based upon new assay data. For a four measurement set the equation is:

Cⱼ=c₀ⱼ+cⱼ₁X₁+cⱼ₂X₂+cⱼ₃X₃+cⱼ₄X₄,

Where there is an intercept and a coefficient for each measurement for each group j, here recent and longstanding. If CRecent>CLongstanding then the new subject is classified as recent and vice versa. This provides an easy to utilize translation from the science and statistical methods to practical utilization.

Results.

Multiplex Incidence Assay Strategy

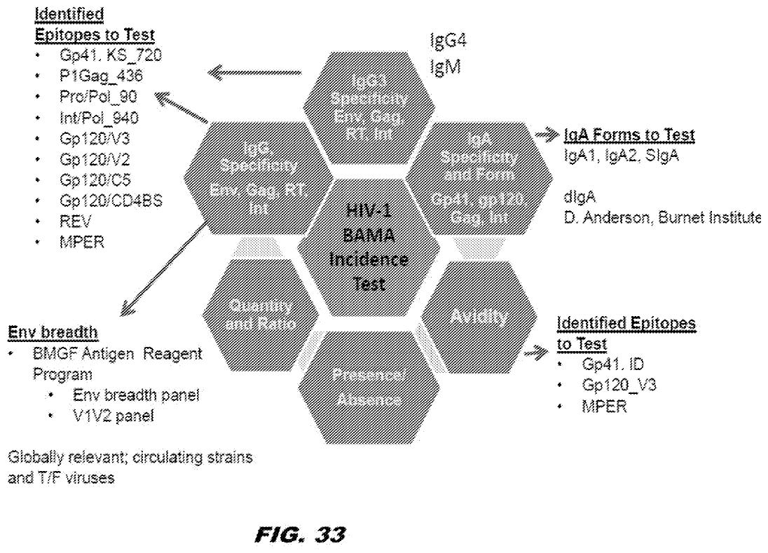

The humoral response to HIV-1 infection is marked by the dynamic appearance and disappearance of certain antibody isotypes and subclasses to different components of the HIV genome from acute to chronic infection. To harness this information for an HIV-1 incidence assay, The inventors tested different antibody forms (IgM, IgG, IgG3, IgG4, IgA) in concert with HIV-1 antigens (peptides and proteins derived from env, gag, pol genes). The analysis includes the presence/absence of the response, the magnitude, and avidity. As part of the discovery phase, The inventors utilized peptide microarray technology to identify linear sequences from the full gp160 envelope that could differentiate recent from longstanding infection. The array of antibody measurements captured as part of this analysis are shown in FIG. 33. The inventors selected an antigen panel that would comprehensively cover the epitopes and antigen structures that would be most likely to be reactive with immune sera from recent to chronic infection, including transmitted founder envelope proteins. The inventors tested samples from recent infection that ranged from 3-266 days post any first positive HIV-1 diagnostic test and samples from longstanding infection ranged from 283 days to >4300 days (Table 1). Estimated date of seroconversion (EDSC) was not known for all longstanding participants.

FIG. 33 Multiplex Incidence Assay Strategy.

Categories of immune measurements that were combined in the multiplex incidence assay strategy.

Table 26. Subtype and Recency Classification of Patient Cohorts.

Cohort characteristics including subtype, recency status and ART are shown. Recent infection was defined as <270 days from either the estimated date of seroconversion or any first positive HIV-1 test (if known) or the maximum time since infected based on Fiebig staging at enrollment [C13]. Samples were characterized as longstanding if >270 days from either the estimated date of seroconversion or from enrollment in an HIV-1 infection cohort. ND=not determined.

HIV-1 Subtype B and C Acute and Chronic Evaluation:

TABLE 26

| | | | ART | EDSC (mean, |
|---|---|---|---|---|
| Classification | Clade | N | use (%, N) | range) |
| Recent | A1 | 11 | 0%, 0 | 96.8, [3, 225] |
| | B | 11 | 18%, 2 | 119.5, [33, 249] |
| | Clade | 15 | 0%, 0 | 147.8, [19, 261] |
| | CRF01_AE | 10 | 10%, 1 | 204.8, [150, 266] |
| | ND | 20 | 20%, 4 | 192, [32, 255] |
| Longstanding | A1 | 1 | 0%, 0 | NA |
| | A1 | 3 | 0%, 0 | 319.3, [143, 529] |
| | B | 39 | 51%, 20 | 1435.0, [117, 4673] |
| | C | 5 | 0%, 0 | 305, [168, 382] |
| | CRF01_AE | 0 | NA | NA |
| | ND | 21 | 5%, 1 | >358 |

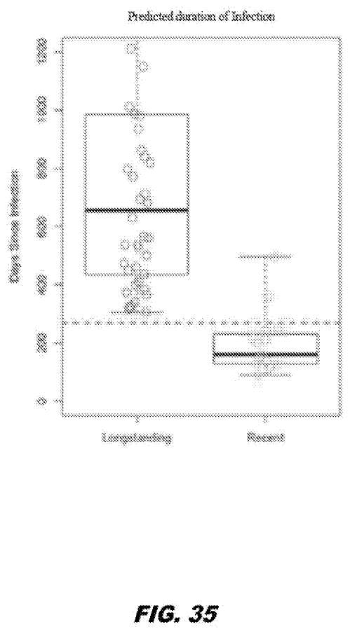

The inventors evaluated plasma from 15 recent and 42 longstanding HIV-1 infections from subtypes B and C infections from the CHAVI 001, and CEPHIA HIV-1 infection cohorts (FIG. 35, Table 27).

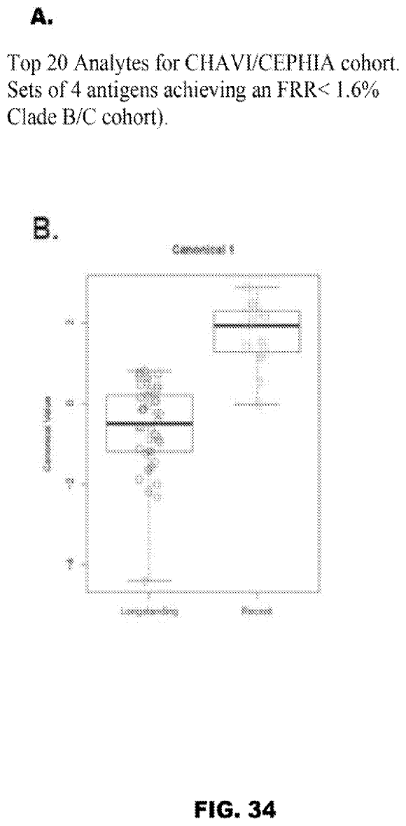

FIGS. 34 and 35 Env Binding and Avidity to Multiple IgG Subclasses Classifies Recent Vs. Longstanding Infection.

An array of 282 antigen-antibody combinations were analyzed using discriminant function analysis. A. The top 20 solutions with an FRR of X, B. Canonical 1 Score for one of the top solutions includes p66 IgG3 avidity, subtype B T/F WITO gp140 IgG4 avidity, gp41 IgM binding, and gp41 IgM avidity.

Table 2Z Discriminant Function Analysis with Best 4 Antigen Set Results in 3.5% Misclassification Rate.

Samples were tested via BAMA and the magnitude of the response for each of the 4 best analytes was analyzed using discriminant function analysis. Group=Classification at 270 days. Assigned group=classification predicted by canonical values obtained via BAMA.

TABLE 27

Assigned Group

| | | Longstanding | Recent | Total |
|---|---|---|---|---|
| Group | Longstanding | 42 | 0 | 42 |
| | Recent | 2 | 13 | 15 |
| | Total | 44 | 13 | 57 |

FRR (T = 2 years) = 0/42 = 0%
Total misclassification = 2/57 = 3.5%

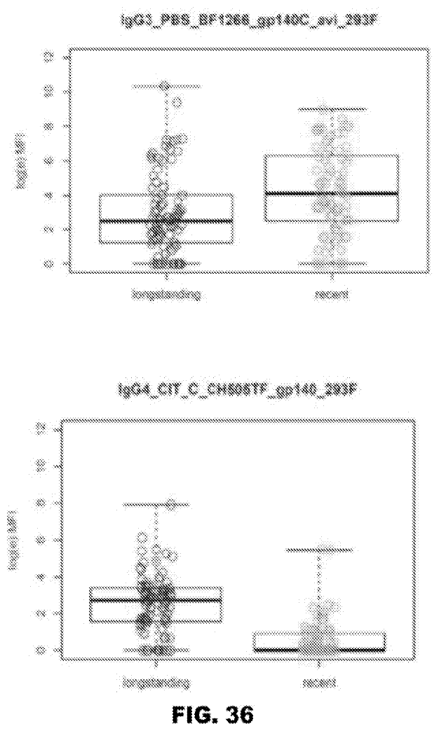

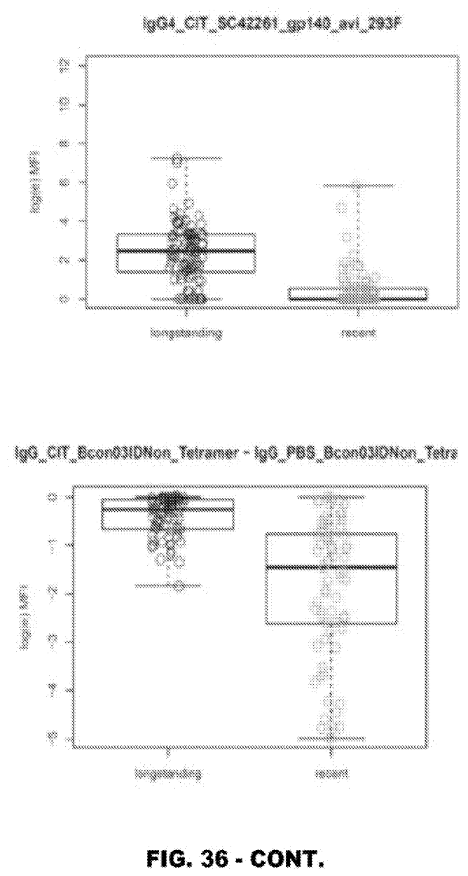

The inventors next evaluated plasma from 70 recent and 66 longstanding HIV-1 infections of multiple subtypes (A, AE, B, C) from the CHAVI 001, RV217, and CEPHIA HIV-1 infection cohorts. Our evaluation included 39 patients on antiretroviral therapy (ART) (Table 1). A recency cutoff of 9 months (270 days) was used for this analysis based upon initial characterization of candidate biomarkers using samples from the CEPHIA DP2 and CHAVI001 acute infection cohorts. To determine the antibody measurements that most accurately categorize patient samples as recent or longstanding HIV infection, the inventors performed discriminant function analysis (DFA) of all possible combinations of 3-6 analytes from 505 possible immune measurements. DFA identified a set of 4 antibody biomarkers that classified recent vs. longstanding infection with a 0% False Recent Rate (T=2 years) and a 6.6% overall misclassification rate (Table 28). As supported by previous observations, Env binding to IgG3 was more strongly associated with recent than longstanding infection (FIG. 36, Panel A). Consistent with a delayed elevation of IgG4 and antibody avidity in HIV-1 infection, Env IgG and IgG4 Env binding avidity were associated with longstanding infection (FIG. 36, Panels B-D). The inventors found that specific antigens were most sensitive for discriminating these responses (i.e. SC42261 and CH505 T/F gp140 for IgG4 avidity and gp41 immunodominant epitope for IgG).

Table 28. A Set of 4 HIV-1 Specific Measurements Predicts Recent HIV-1 Infection with Low Total Misclassification Rate.

Samples were tested via BAMA and the magnitude of the response for each of the 4 best analytes was analyzed using discriminant function analysis. Group=Classification at 270 days. Assigned group=classification predicted by canonical values obtained via BAMA.

TABLE 28

Assigned Group

| | | Longstanding | Recent | Total |
|---|---|---|---|---|
| Group | Longstanding | 63 | 3 | 66 |
| | Recent | 6 | 64 | 70 |
| | Total | 69 | 67 | 136 |

FRR (T = 2 years) = 0/66 = 0%
Total misclassification = 9/136 = 6.6%

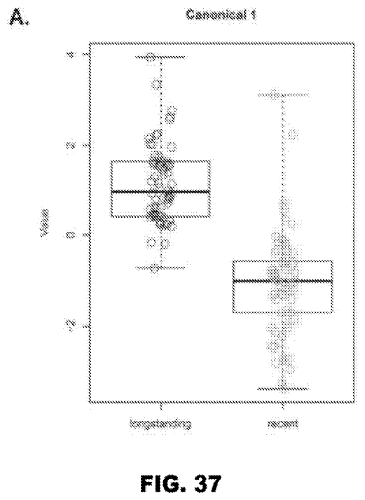

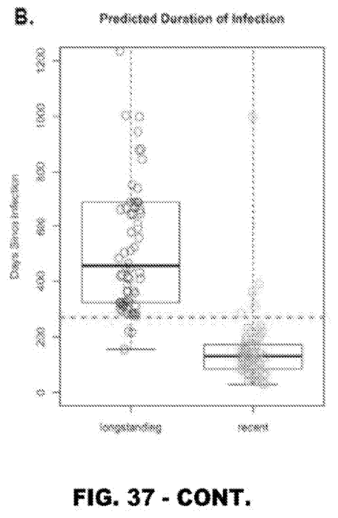

Responses to the four individual biomarkers exhibited overlap between recent and longstanding specimens, therefore the inventors used discriminant function analysis to generate a canonical score for each specimen (FIG. 37A). The canonical score was then used to calculate predicted duration of infection (PDI) for each specimen and to classify each specimen as recent or longstanding (FIG. 37B). This set of four biomarkers achieved low overall misclassification rates as follows: 3/69 (4.5%) of longstanding were misclassified as recent, and 6/70 (8.6%) of recent specimens were misclassified as longstanding using a cutoff of 270 days. Total misclassification rate, including specimens on ART was 9/136 (6.6%) (Table 28).

FIG. 37 Canonical Value Determination Accurately Predicts Estimated Duration of Infection.

A. a canonical value was calculated for each sample based upon responses to the 4 analytes. B. Predicted duration of infection was calculated based upon the canonical value obtained for each sample. A canonical value of <0 indicates recent infection and a canonical value >0 indicates longstanding infection.

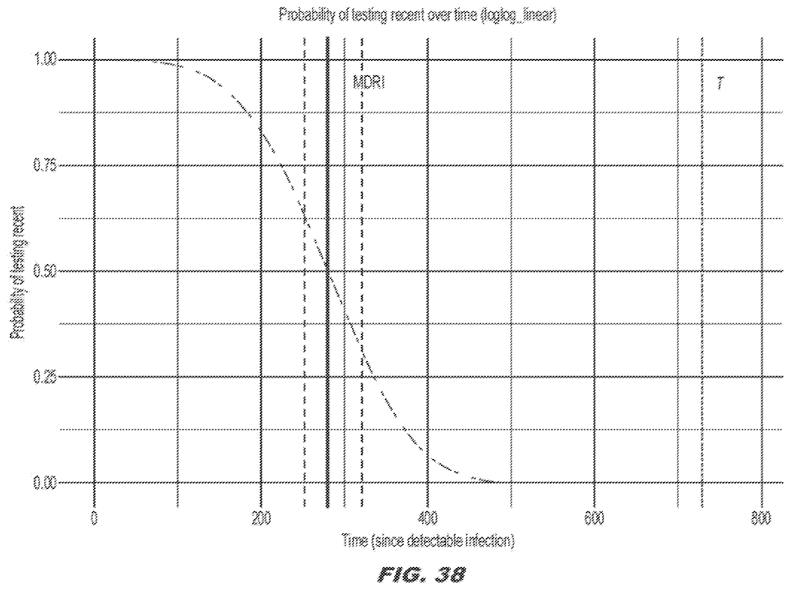

The inventors calculated the MDRI for the best 4 set of antibody biomarkers using Canonical 1 in the threshold model (described by Welte, et al). The estimated MDRI for the best 4 antibody biomarkers (BF1266 gp140 IgG3 binding, CH505 TF gp140 IgG4 avidity magnitude, SC42261 gp140 IgG4 avidity magnitude, and the difference score of gp41 ID IgG epitope binding and avidity magnitude) is 279 days (95% CI=250.3, 320.4) (FIG. 38) and FRR (T=2 yrs) is 0% (Table 28).

FIG. 38 MDRI Estimate and Confidence Intervals for Novel BAMA Biomarker Panel.

MDRI was calculated using [INSERT method here]. Results are presented as the probability of testing recent over time, with 95% confidence intervals presented as dashed blue vertical lines. The cutoff for FRR (T=2 years) is indicated with a red vertical line.

All samples were evaluated by LAg assay (SEDIA) to compare BAMA classification accuracy to a standardly used, commercially available incidence assay. Table 5 presents results of the LAg assay (ODn) and BAMA canonical score, along with resulting specimen classification per assay. For the purposes of this comparison, samples on ART were included in calculations of false recent and false longstanding rates. BAMA accurately classified X/Y64/70 recent specimens (Recent) and X/Y52/54 longstanding specimens (Longstanding) with low False Recent (%) (3.7%) and False Longstanding rates (%) (8.6%) based on a recency cutoff of 270 days (Table 5). Using the published maximum MDRI of 211 days for the LAg [C6], the assay accurately classified X/Y42/55 recent specimens (Recent) and X/Y37/51 longstanding specimens (Longstanding). False recent rates and false longstanding rates for the LAg based on a recency cutoff of 211 days were 14% and 23.6%, respectively.

Table 29. LAg and BAMA Comparison.

LAg testing was performed to compare with BAMA results.

TABLE 29

Assigned Group

| | | Longstanding | Recent |
|---|---|---|---|
| BAMA | Longstanding | 52 | 2 |
| | (≥270 days) | | |
| | Recent | 6 | 64 |
| | (<270 days) | | |
| LAg | Longstanding | 37 | 14 |
| | (≥197 days) | | |
| | Recent | 13 | 42 |
| | (<197 days) | | |

Additional testing was also performed to assess the potential presence of anti-retroviral agents in plasma (CEPHIA panels, CHAVI 001) and in urine (HRBS panel). A simple testing method for the presence of antiretrovirals may further improve the specificity of a new HIV-1 incidence test. One of the negative controls in standard neutralization assays is the measurement of neutralization of a reporter virus (ie. MLV) that lacks the HIV envelope. Although MLV cannot be neutralized by HIV specific antibodies, inhibition can occur in the presence of antiretrovirals. For the purposes of this analysis, samples were defined as positive ART if the MLV neutralization titer was greater than 3× the negative threshold (i.e. a titer>60). Results of this analysis indicated that 3/28 (10.7%) samples scored negative for MLV neutralization when ART use was reported and that 3/227 (1.3%) samples scored positive for MLV neutralization when the participant was reported to be off ART.

### Discussion

Accurate determination of the effectiveness of HIV prevention and treatment programs requires assays that can discern and quantify new infections. Large scale investments in HIV prevention efforts are dependent on the reliability and accuracy of incidence assays. Clinical trials for new therapeutics and prevention interventions are based on defined endpoints of reducing the number of new infections. Thus, decisions for prevention implementation, in predominantly low income countries with the highest burden of global and HIV and AIDS, rely on the accuracy of these assays. The accurate measurement of new infections is currently plagued by the lack of assays that can reliably be utilized in high incidence African countries due to the complexity of discerning between recent and longstanding infections. The complexity stems from the diversity in antibody responses among individuals and among HIV subtypes, genetic diversity in virus across regions, and HIV disease state and ART use that results in suppressed antibody responses. Altogether, these variables make the development of a standard global HIV incidence assay difficult and highlight the critical need for innovative methods to identify new infections in order to inform decisions on the large scale roll-out of new prevention strategies.

Improvements in HIV-1 incidence estimation can be achieved through combining multiple tests into a Multi-Assay Algorithm (MAA), including antibody measurements, p24 antigen, viral diversity and/or viral load determination. Recency Incidence Testing Algorithms (RITA) including STARHS (REF), have also been used in combination to refine estimates of incidence, with varying degrees of success [C6, C19]. However, utility of these assays may be limited due to increased costs per sample, sample volume required, and complexity of the assay. A recent advance in ELISA based assays is the commercially available Limiting Antigen (LAg) avidity assay [C17] that utilizes avidity to a multi-clade recombinant gp41 ID epitope (rIDR-m) and achieves a low FRR of 1.3% when elite controllers and patients on ART treatment are excluded [C6]. Results of an independent analysis of five standard incidence assays, including LAg, (BioRad avidity, Vitros, LS-Vitros, LAg and BED) indicated a high false recent rate of 12.9-48.4% in elite controllers and 50.0-76.1% in ART treated individuals, indicating the need for novel biomarkers of HIV-1 incidence.

Considerations for novel development of HIV-1 incidence biomarkers must include expansion of the range of biomarkers to achieve greater separation between recent vs longstanding specimens. Current assays are limited in Env sequence, clade diversity, antibody isotypes and rely on non-native protein sequences for assessment of binding antibody responses. A majority of currently available incidence assays measure avidity to a limited number of sequences, including varying recombinant forms of gp41 IgG (LAg, BED (REFS)), multiple fusion proteins comprised of varying combinations of gp120/gp41, p24 and p36 (Vitros and LS-Vitros, REFS) and gp160 and p24 recombinant proteins derived from HIV-1, gp36 from HIV-2 and a synthetic polypeptide mimicking an artificial HIV-1 group 0 specific epitope (Bio-Rad avidity, REF). Recent assays have begun to include binding and avidity to other Env and non-Env sequences such as p66 and gp120 [20], which display a range of maturation kinetics from recent to longstanding infection. However, addition of novel biomarkers such as clade diversity and maturation kinetics of antibody isotypes and subclasses are likely to provide the greatest reductions in FRR. Thus, the inventors pursued an expanded set of novel, multiplexed antibody biomarkers that included: binding and avidity to a range of linear epitopes, diverse Env and non-Env proteins (native and consensus sequences) and Ig isotypes and subclasses. This work built upon previous observations that IgG subclasses and Env specificities display differential kinetics during the course of recent infection [C8, C9]. The panel of antigens was built to balance potential antibody biomarkers of recency (gp41 epitopes, dIgA, IgG3, IgM) with markers of longstanding infection (gp120 epitopes, IgG4, avidity to gp41). This panel also included maximal diversity through addition of globally relevant, circulating strains and transmitted founder Env proteins from the BMGF Antigen Reagent Program (deCamp, Yates, Tomaras, in preparation). Additionally, the inventors tested whether an MLV neutralization test could provide additional information on the presence of ART in plasma or urine specimens. One caveat of the approach tested here is that ART use was not experimentally measured in each sample to confirm true presence or absence of drug. The results here, although not definitive, suggest that such an approach of testing for ART use by an in vitro virological assay could have application as part of an HIV-1 incidence algorithm.

In conclusion, the inventors have identified multiple subsets of HIV-1 specific Ag/Ab pairs that distinguish recent (<9 months) vs. longstanding infection in a globally relevant panel with a 0-4.5% false-recent misclassification rate, that notably included ARV treated patients. These data compare favorably with current incidence assays that misclassify between 12.9%-76.1% of elite/ARV treated patients. Thus, the biomarkers of HIV-1 recency reported here are excellent candidates to advance for further development of a novel HIV-1 incidence algorithm.

TABLE 30

Assigned group

| | | On ART | No ART | Total |
|---|---|---|---|---|
| MLV | On ART | 25 | 3 | 28 |
| neut | No ART | 3 | 224 | 227 |

### Example 7: Development of a Novel Antibody Biomarker Assay for Global HIV-1 Incidence Determination

Current Incidence Assays

(See e.g. Schlusser et al. PLoS One. 2017 Feb. 23; 12(2):e0172283. doi: 10.1371/journal.pone.0172283. eCollection 2017), for example, (a) BED (CDC): Proportion of IgG specific to HIV, gp41 ID region, gp41 ID region; (b) LAg (CDC): Limiting antigen avidity assay (gp41 ID); (c) Less-sensitive Vitros/Vitros avidity: 4 recombinant proteins (gp120/gp41; p24); (d) Bio-Rad avidity (see e.g. Hauser et al. (2014) PLoS ONE 9(6): e98038. doi:10.1371/journal.pone.0098038): modification of the Genetic Systems HIV-1/HIV-2 plus 0 EIA; and gp160, p24; (e) Bio_Rad Geenius HIV1/2 Supplemental Assay for Detecting Recent HIV Infection and Calculating Population Incidence (see e.g. Keeting et al. J Acquir Immune Defic Syndr. 2016 Dec. 15; 73(5):581-588.)—antibody index (p31+gp160+gp41/control band: diversity and quantity Index) to determine titer and specificities of anti-HIV Ab.

Binding Antibody Multiplex Assay (BAMA)

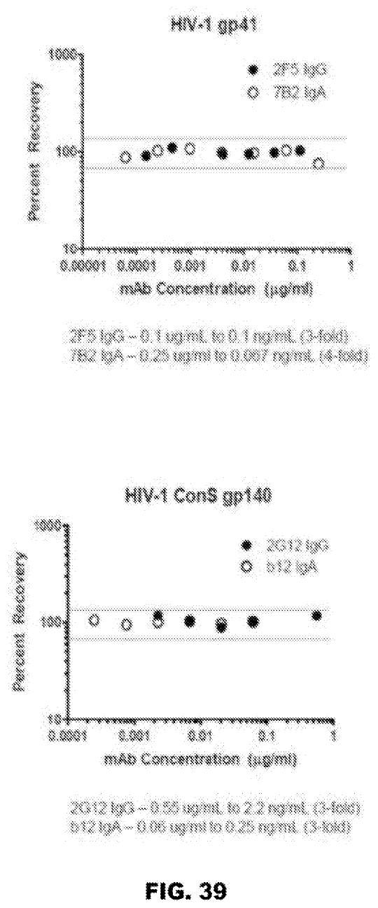

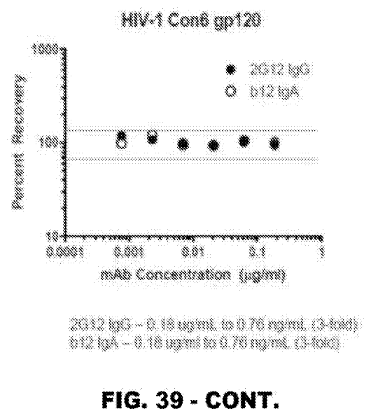

BAMA Accuracy and Linearity (FIG. 39).

Purified HIV-1 specific monoclonal antibodies were spiked into pooled and diluted seronegative plasma as indicated below, and incubated with antigen-coated beads. Accurate recovery was determined according to the preset recovery criteria (70-130% of expected concentration).

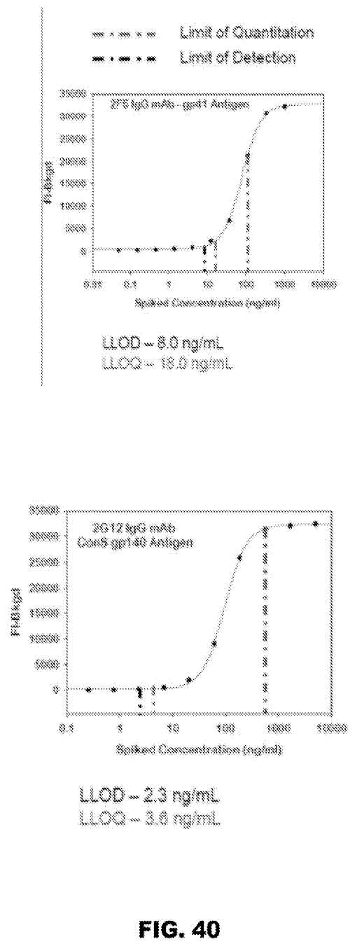

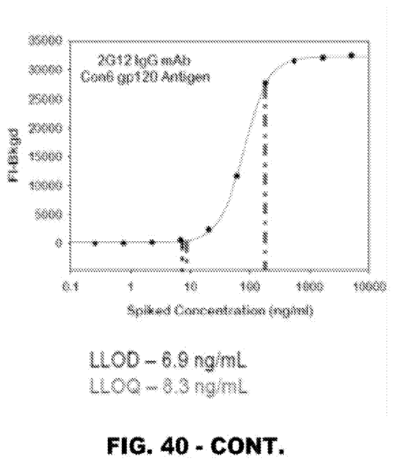

Limit of Detection/Quantitation (FIG. 40).

Purified HIV-1 specific monoclonal antibodies were spiked into pooled and diluted seronegative plasma as indicated below, and incubated with antigen-coated beads. LLOD and LLOQ were determined based upon antigen-specific background reactivity with at >30 seronegative plasma samples.

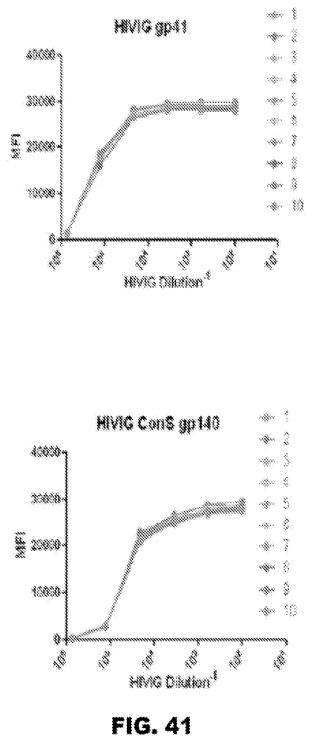

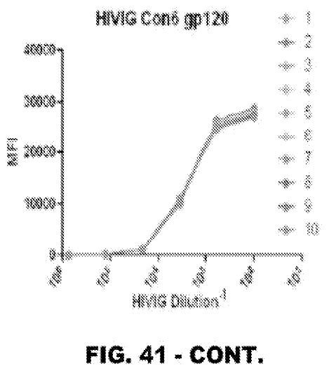

BAMA Repeatability (FIG. 41).

HIV-1 BAMA results are highly repeatable across multiple assays. Purified pooled HIV-1+IgG (HIVIG) was titrated in BAMA assay diluent and ten separate binding antibody curves were generated to gp41, ConS gp140, and Con6 gp120. Individual binding curves the 10 replicate titrations are overlaid.

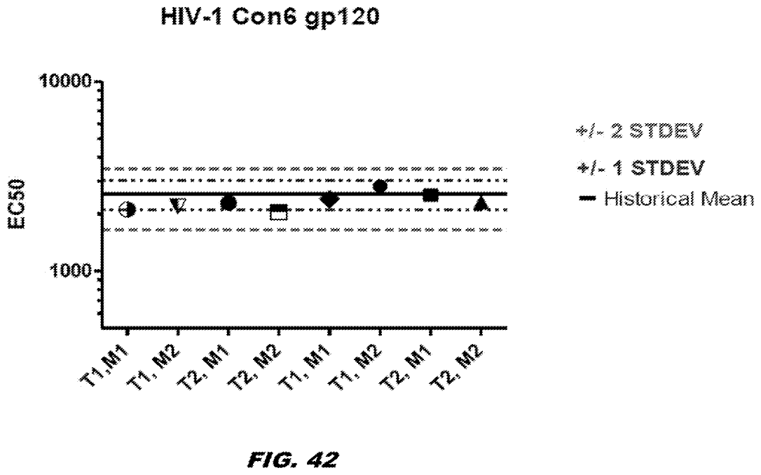

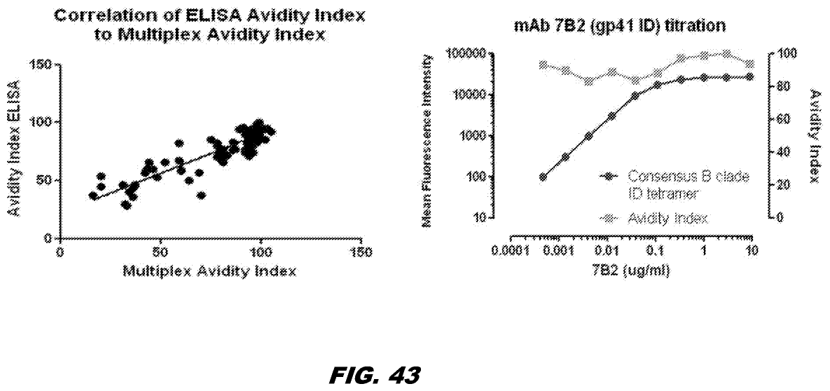

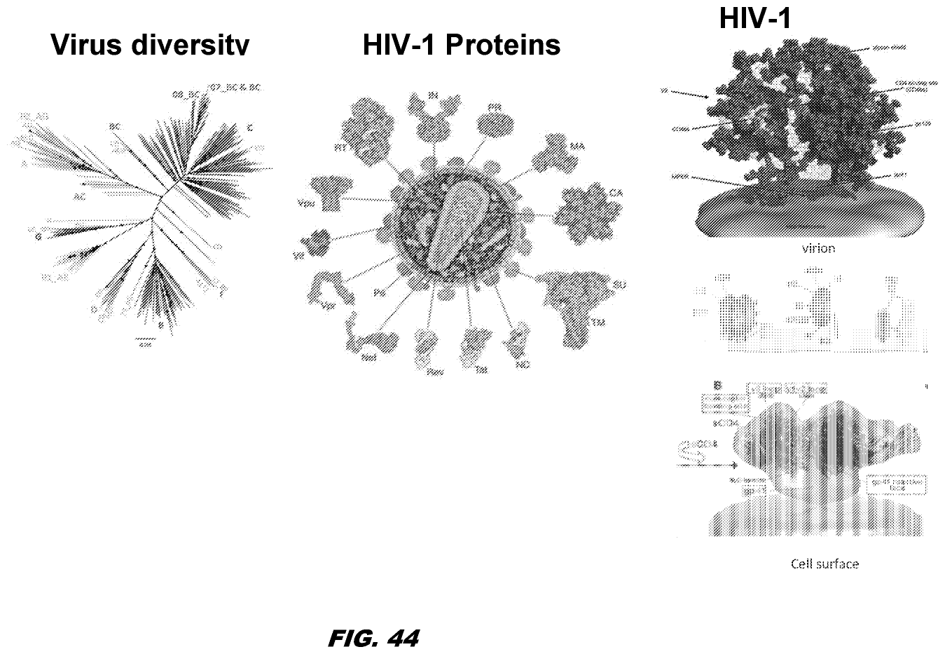

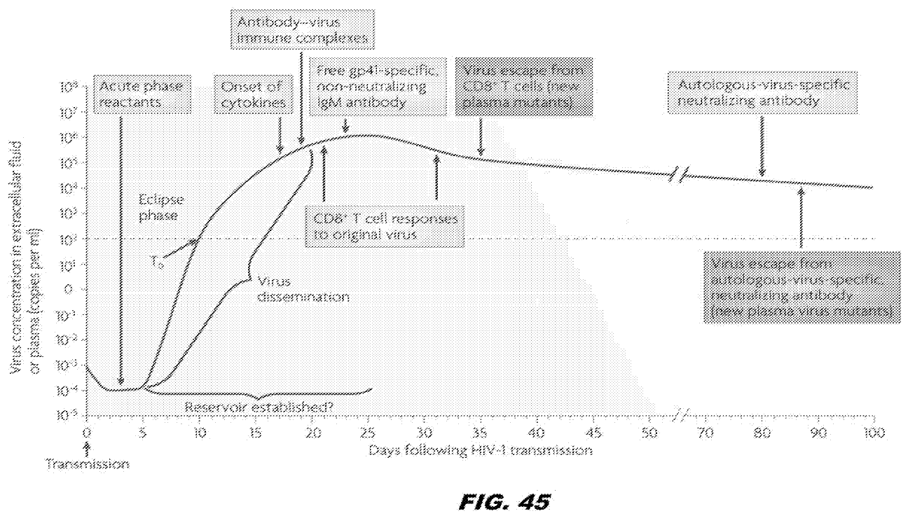

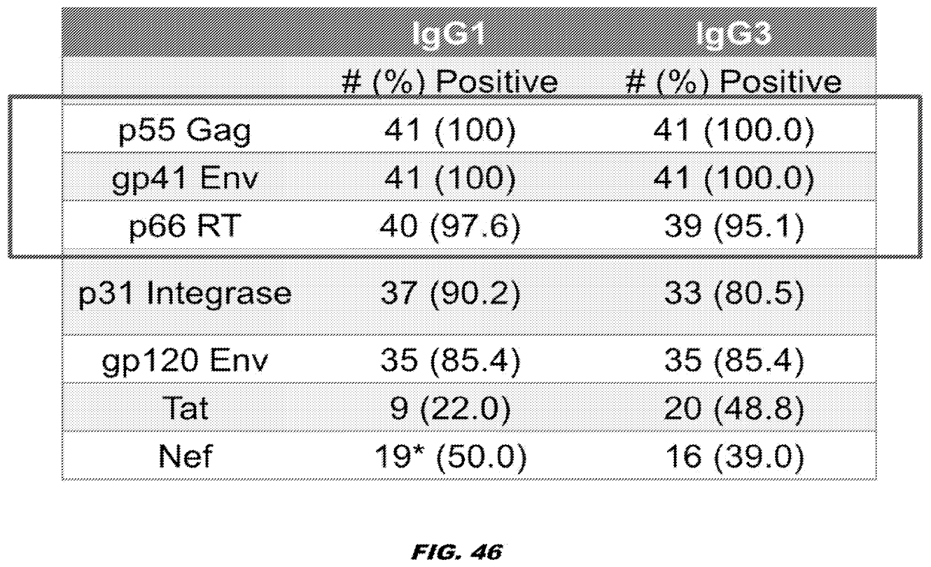

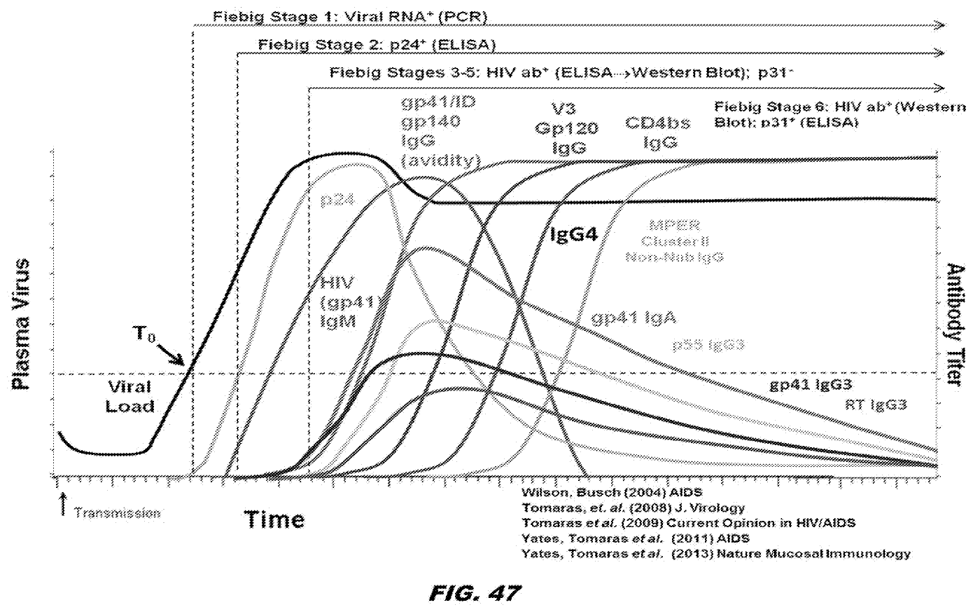

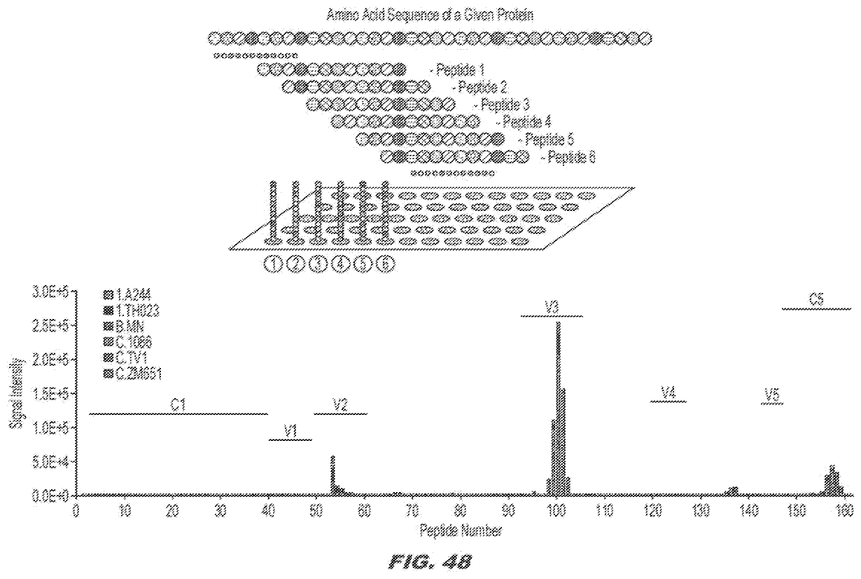

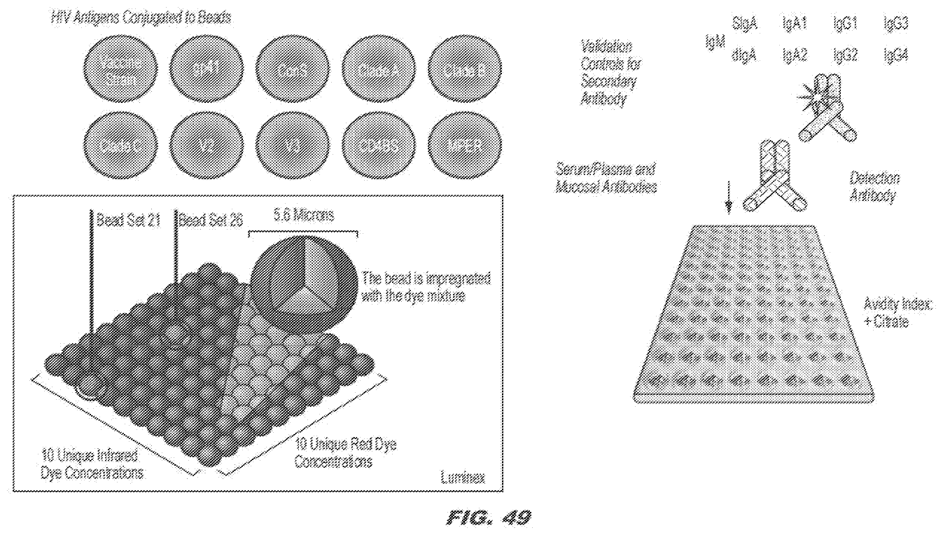

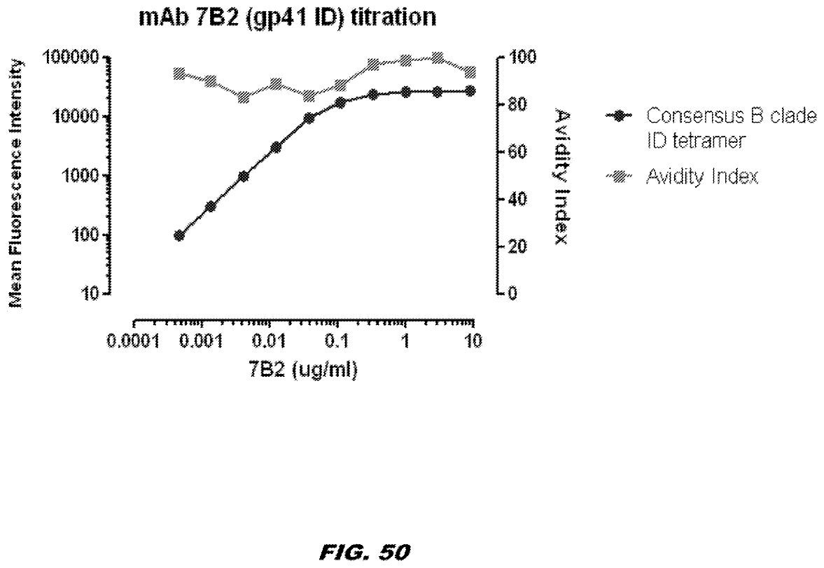

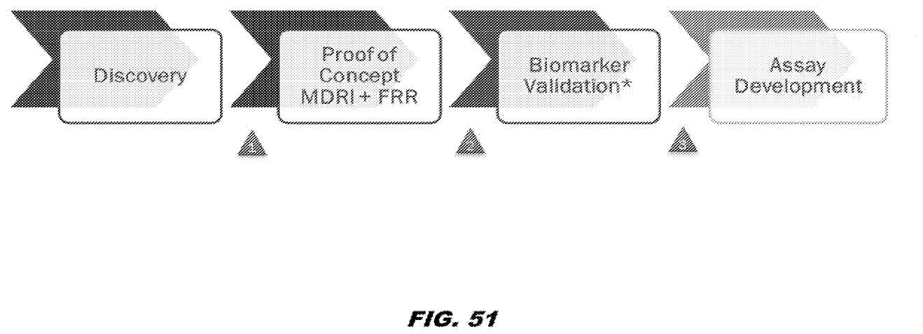

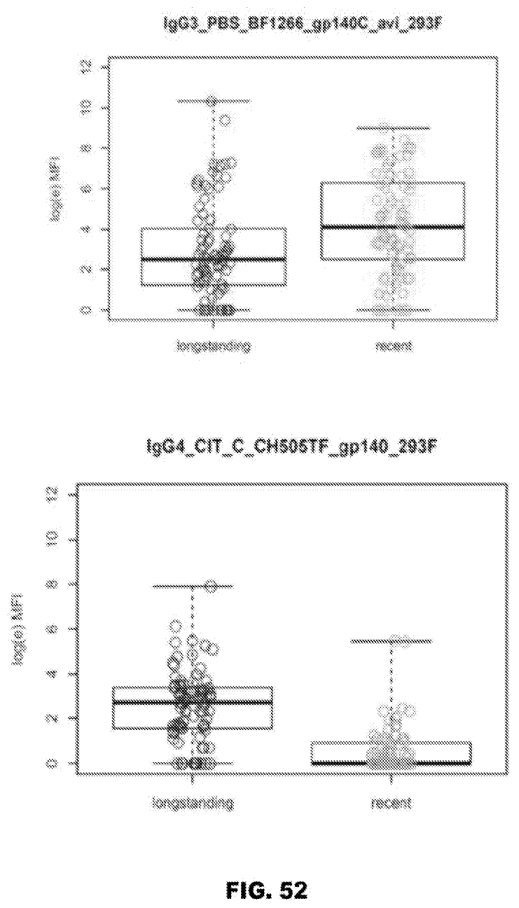

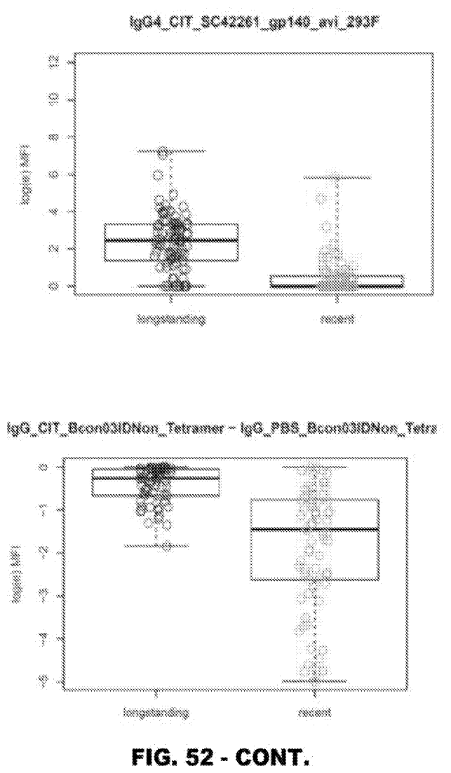

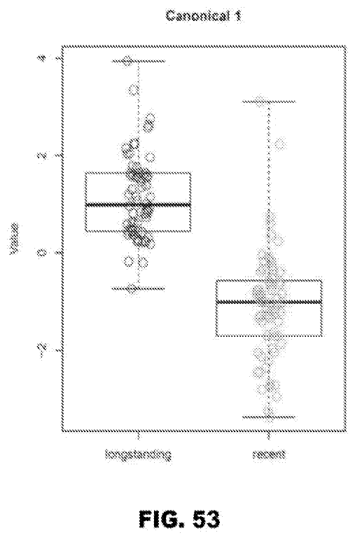

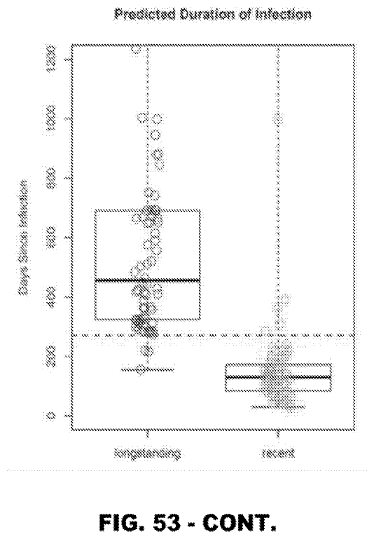

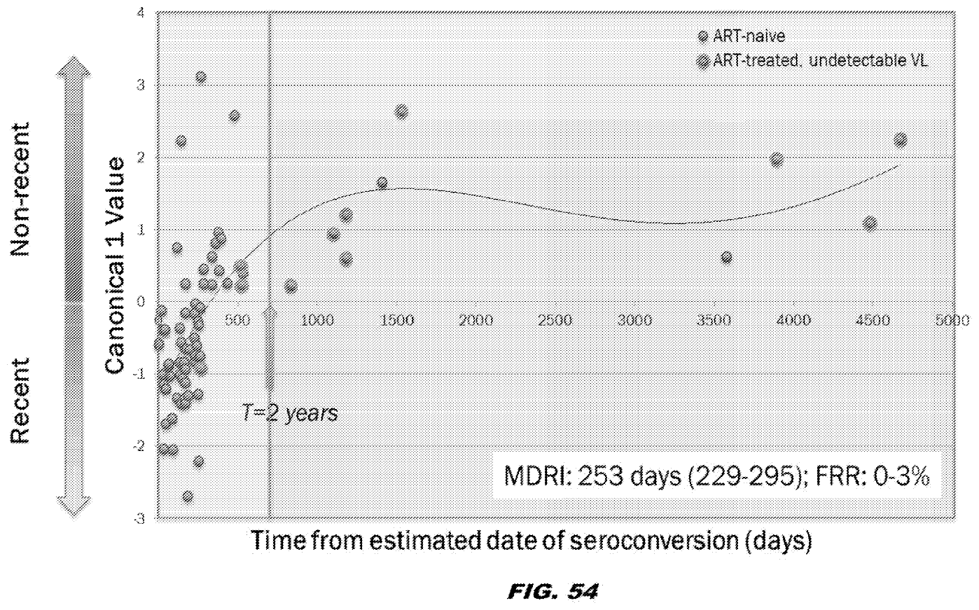

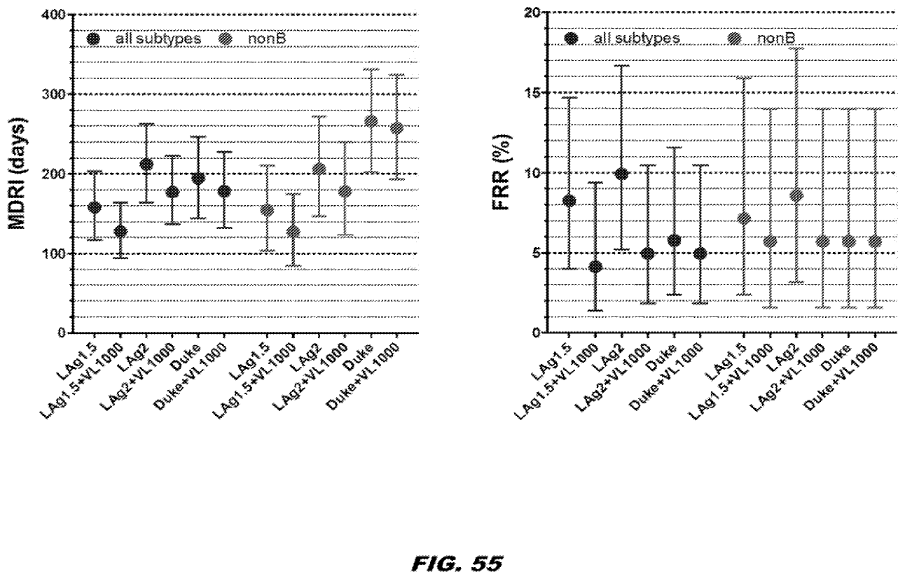

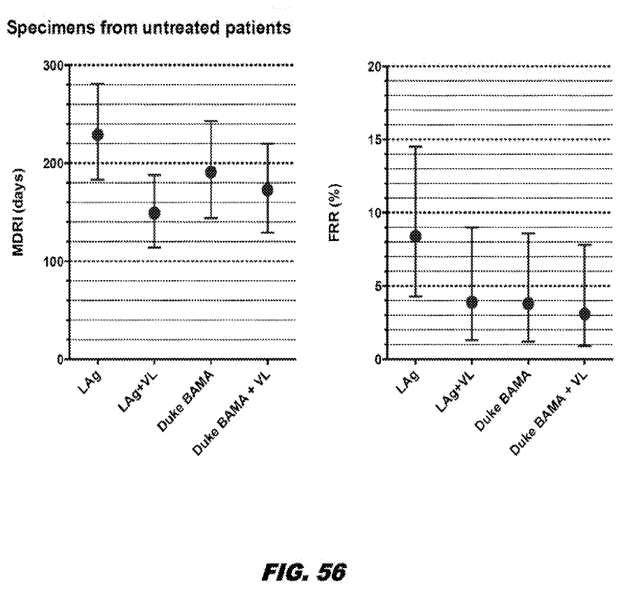

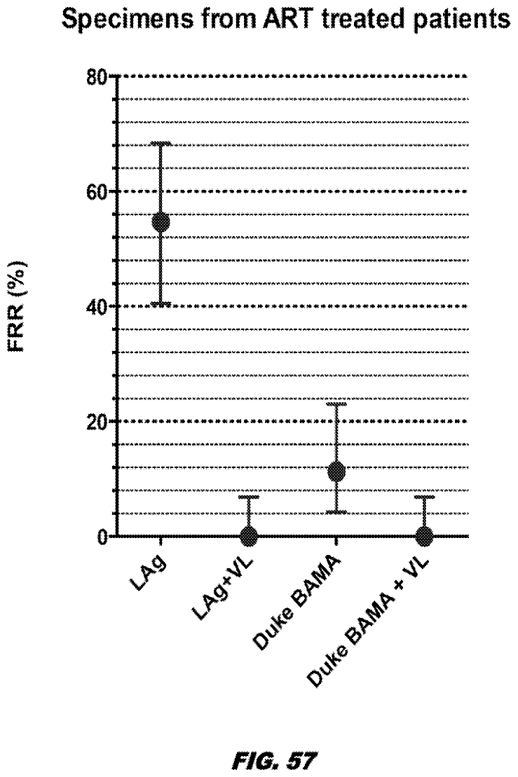

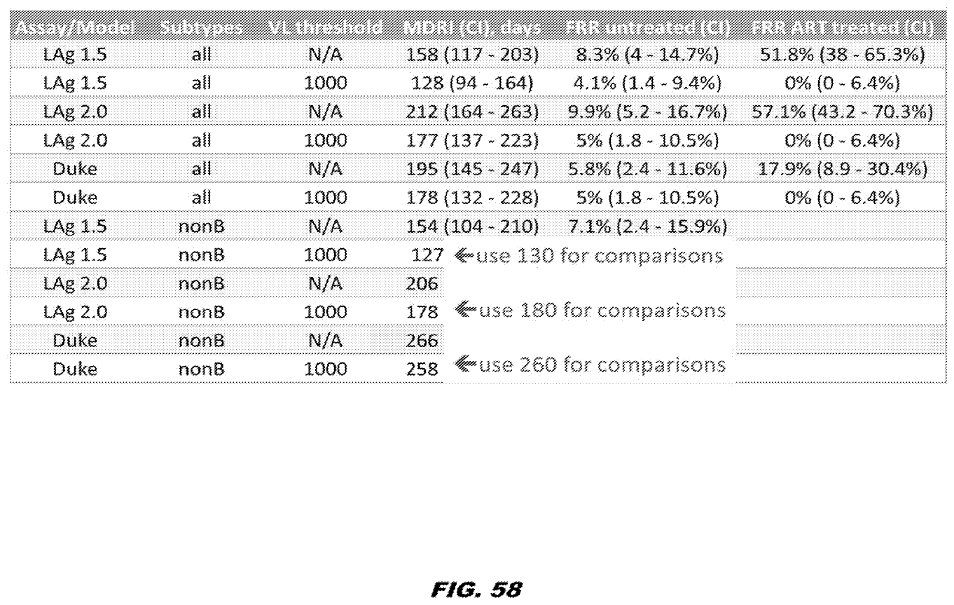

Levey Jennings Antigen Reagent Tracking (FIG. 42).

Intermediate Precision—IgG EC₅₀ Titers remain consistent over operators, instruments, and time Purified pooled HIV-1+IgG (HIVIG) was titrated in BAMA assay diluent and subsequently incubated with Con6 gp120 coupled beads. This experiment was performed separately by two technicians on consecutive days, and each HIVIG curve was read on two Bioplex 200 instruments. T1/T2=Technician 1/Technician 2; M1/M2=Machine 1/Machine 2.

BAMA Avidity Index (FIG. 43).

2-well format; 15 minute dissociation with Na-Citrate, pH 3.0; Avidity Index=MFI(CIT)/MFI(PBS)*100. Antibody biomarkers of acute infection alone or in combination may reduce false recent rate (FRR). These antibody biomarkers may also have a reasonably long Mean Duration of Recent Infection (MDRI). 6-12 months ideal (Incidence Assay Critical Path Working Group, PLOS Medicine, 2011). May be either presence or absence of specific markers.

Pitt Panel 2

Data Analyzed—Proof of Concept Panel (Pitt Panel 2)
505 Antibody-Antigen Measurements
   Envelope (gp41, gp120, gp140), and Non-Env Antigens
   peptide epitopes (including ID and new epitope discovery from peptide microarrays)
   IgG, IgG3, IgG4, mIgA, dIgA, IgM
   Output measurements:
      Magnitude (MFI)
      Dilution
      Avidity (Binding in presence of Na-Citrate pH 3.0)

Evaluation/Testing: CHAVI 001, RV217, Pitt Panel 2, CEPHIA DP2

Combination Panel—Characteristics

TABLE 31

| | CEPHIA | CHAVI | | | |
|---|---|---|---|---|---|
| | DP2 | 001 | Pitt Panel 2 | RV217 | Total |
| Longstanding | 10 | 24 | 32 | 0 | 66 |
| Recent | 0 | 25 | 28 | 17 | 70 |
| Total | 10 | 49 | 60 | 17 | 136 |

Equal numbers of recent (<9 months)/longstanding
Multi-clade (A, A/E, B, C)
Tested downselected “best antigen” sets from Pitt Panel 2 analysis
Isotypes: IgA, IgG, IgG3, IgG4, IgM, pIgR
Binding and avidity measurements (avidity measurements—Parekh, et al 2012)

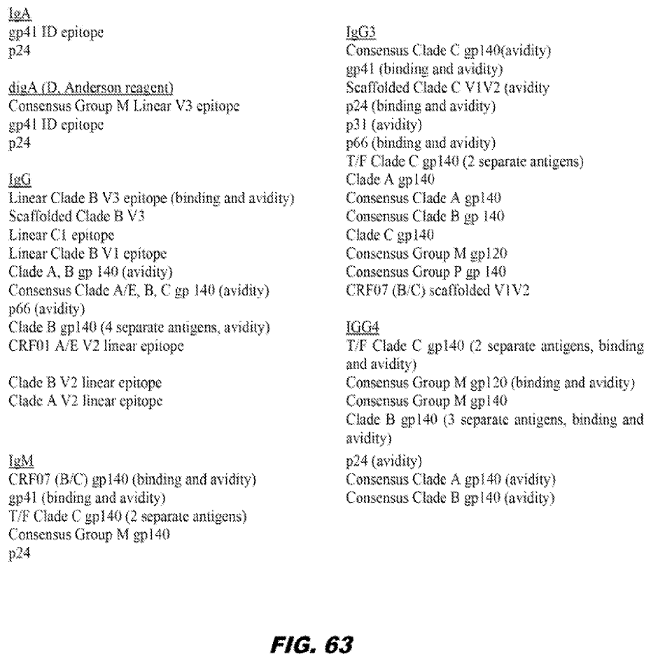

Downselected Antigen Panel (See FIG. 63)

Methods for Down-Selection (Combined Panel)

1. Use each measurement as a continuous predictor of Classification in a logistic regression.
2. Use each measurement as a categorical predictor of Classification in a logistic regression.
3. Rank order measurements by the absolute value of the correlation with EDSC.
4. Rank order measurements by the mean difference between Classification.
5. Rank order measurements by the difference in positive response rate.
6. Combine the order score for methods 1-5.
7. Use the pairwise difference as a continuous predictor of Classification in a logistic regression.

Algorithm for Determining “Best” Measurements

For each of the first 6 methods: (1) We ran a discriminant function analysis (DFA) for all possible sets of 3 to 5 measurements. (2) We recorded the misclassification error rate for the solution for all possible sets. (3) We determined the lowest error rate for each method by measurement combination and output the measurements in that solution.

For difference scores, a DFA was run on the top difference scores that had the lowest correlations.

The best combination of difference scores were then combined with the top model measurements using a stepwise approach.

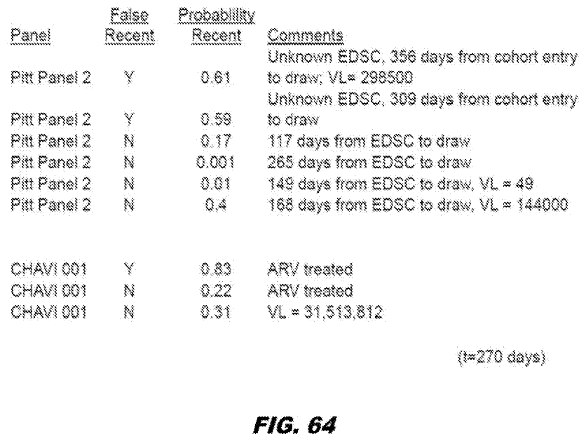

### Misclassified Specimens (See FIG. 64)

### Conclusions

We have identified multiple subsets of HIV-1 specific antigen/antibody pairs that are excellent candidates for determination of recent (<9 months) vs longstanding infection. Additionally, a subset of 4 direct binding and antibody avidity measurements are able to distinguish recent (<9 months) vs longstanding infection in a globally relevant panel with an FRR=0% including samples from patients treated with ARV. Currently available incidence assays exclude elite controllers or ARV when estimating overall assay FRR due to poor performance with these difficult to classify specimens (FRR from 12.9%-76.1% in elite/ARV treated). Thus, the novel antibody measurements are excellent candidates to advance for further development of a novel HIV-1 incidence algorithm.

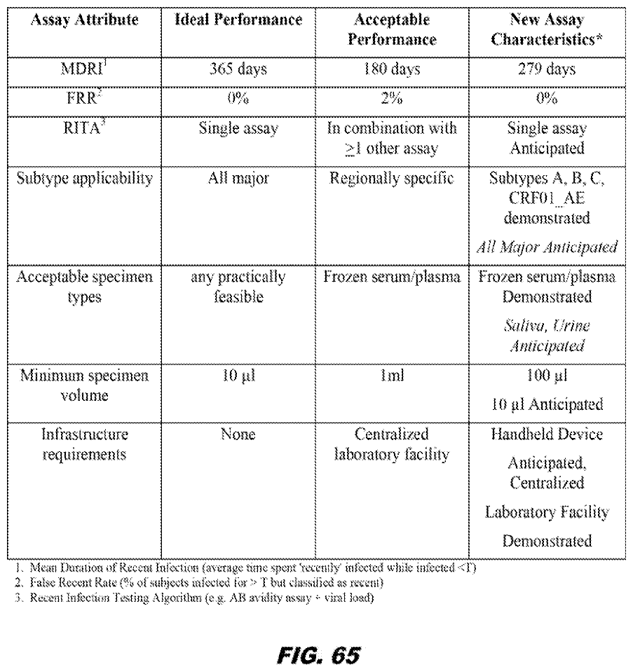

Draft Target Product Profile (BMGF, FIND) (See FIG. 65)

### Example 8: Harnessing Antibody Responses as HIV-1 Incidence Biomarkers

This is example provides: (a) a foundation for antibody measurements for HIV-1 incidence tests; (b) Technology/Methods for defining measurements; and (c) Results of Discovery and Proof of Concept Tests.

Rationale for Duke HIV-1 Incidence Assay.

The natural kinetics and maturation of the antibody response after HIV-1 infection can be harnessed for HIV-1 incidence assays: Can have sequential recognition of different HIV-1 proteins post infection; Can observe Dynamic appearance of HIV-1 antibody isotypes (IgM, IgG, IgA) and subclasses (IgG3, IgG4) post infection; and can observe the B cell response to infection results in the maturation of the antibody response resulting in increasing avidity for the target.

TABLE 32
Timing of the Initial Anti-Env Response (Tomaras, et al. (2008) J.
Virology, 82: 12449).

| | | Median | 0.95 | 0.95 | |
|---|---|---|---|---|---|
| Antigen | N | Time^{a} | LCL | UCL | Range |
| gp41 | 19 | 13 | 12 | 14 | (9-18) |
| gp140 | 19 | 13 | 12 | 15 | (6-17) |
| ID gp41 peptide^{b} | 13 | 18 | 16 | —^{c} | (13-34) |
| gp120 | 7 | 28 | 26 | — | (13-41) |
| V3 | 7 | 34 | 19 | — | (13-36) |
| Non-neutralizing MPER, | 19 | >40 | — | — | — |
| CD4BS, CD4i | | | | | |

^{a}Median time from T0, first day viral load reaches 100 copies/ml.
^{b}ID = immunodominant
^{c}Not determined since some of these antibody responses arise greater than >40 days from T₀

TABLE 33
Timing of HIV-1 IgA Responses (Yates, Tomaras (2013) Nature Mucosal Immunology 6(4): 692-703).
Table 1 Timing of systemic and mucosal gp41-specific IgA antibodies

| | gp41 | | Gag | | BT | | Nef | | gp120 | | P31 | |
|---|---|---|---|---|---|---|---|---|---|---|---|---|
| Fiebig stage | No./total^{a} | % | No./total | % | No./total | % | No./total | % | No./total | % | No./total | % |

(a) Plasma IgA

| | | | | | | | | | | | | |
|---|---|---|---|---|---|---|---|---|---|---|---|---|
| I/II | 6/23 | 26.1 | 2/23 | 8.7 | NT | — | NT | — | NT | — | NT | — |
| III | 7/14 | 50.0 | 3/14 | 21.4 | NT | — | NT | — | NT | — | NT | — |
| IV | 15/17 | 88.2 | 10/17 | 58.8 | 3/7 | 42.9 | 3/7 | 42.9 | 2/8 | 25.0 | 0/7 | 0.0 |
| V/VI | 39/40 | 97.5 | 35/40 | 87.5 | 13/15 | 86.7 | 8/14 | 57.1 | 11/29 | 37.9 | 3/14 | 14.3 |

(b) Mucosal IgA

| | | | | | | | | | | | | |
|---|---|---|---|---|---|---|---|---|---|---|---|---|
| I/II | 0/1 | 0 | 0/1 | 0 | NT | — | NT | — | NT | — | NT | — |
| III | 0/2 | 0 | 0/2 | 0 | NT | — | NT | — | NT | — | NT | — |
| IV | 7/21 | 33.3 | 4/8 | 50.0 | 0/7 | 0 | 0/7 | 0 | 1/7 | 14.3 | 0/7 | 0 |
| V/VI | 18/23 | 78.3 | 17/23 | 73.9 | 5/14 | 55.7 | 1/14 | 7.1 | 4/14 | 28.6 | 1/14 | 7.1 |

| | | |
|---|---|---|
| Antigen | No. of subjects^{b} | Median time to antibody response (range (days))^{c} |

(c)

| | | |
|---|---|---|
| gp41 | 14 | 13.5 (9-18) |
| p55 | 8 | 25.5 (14-40) |

HIV-1 Envelope Proteins.

New well-characterized cross-clade gp120, gp140 and V1V2 proteins to cover subtypes AE, A, B, BC, and C from diverse geographic regions (Malawi, China, Thailand, South Africa, US, India, Zambia, Uganda, Trinidad, Kenya)

Protein production by Duke protein production facility (PPF).

Well characterized (SPR, FPLC) HIV-1 envelope glycoprotein antigenicity.

Incidence Assay Based on Core Validated Assay for HIV-1 Immune Monitoring Studies.

Provide GCLP-compliant, Validated Binding Assays (with established SOPs) to: (1) HIV Vaccine Trials Network (HVTN), NIH; (2) Collaboration for AIDS Vaccine Discovery (CAVD), BMGF; (3) Center for HIV/AIDS Vaccine Immunology & Immunogen Discovery (CHAVI-ID), NIH; and (4) Duke Center for AIDS Research Immunology Core (CFAR)

Recent Assay Training and Technology Collaborations (Training and Competency SOPs Established): (1) Pediatric HIV/IMPAACT Laboratories (NIH); and (2) CAPRISA Laboratories (South Africa).

Audits and Oversight: (1) Duke Central Quality Assurance Unit; (2) NIH DAIDS yearly audit for HVTN; and (3) College of American Pathologists (CAP) Certification.

Improving HIV-1 Incidence Assays.

Current HIV-1 Incidence assays focus on IgG detection/avidity of immunodominant gp41. The inventors have identified a series of new biomarkers as potential indicators of HIV-1 incidence: (a) Antibody Isotypes and Subclasses: IgG, IgG3, IgG4, IgA; and (b) HIV-1 Proteins and Epitopes: Envelope and non-Envelope,

Terminology.

Biomarker Discovery/Downselection Steps:

Threshold for recency: a cutoff in time since EDSC used to classify specimens during model training (e.g. by minimizing mis-classification rate)

no fixed “best” threshold a priori

not equivalent to, but likely to have significant impact on, MDRI

“recent” mis-classification % not the same as FRR

Recency Classifier Evaluation:

(1) T: fixed point in time used to define MDRI and FRR; current consensus is to use 2 years; (2) MDRI: average time spent classified as ‘recently’ infected while infected for less than T; (3) FRR: probability that a person who is infected for longer than T will return a ‘recent’ result.

Cohort Characteristics.

Cephia Repository:

Development Panel 2—Clade B; and Pitt Panel 2—Clades A, B, C; samples on ART

CHAVI 001:

Clades B, C; samples on ART

RV217:

Clades A/E, B; Recent infection cohort

TABLE 34
Cohort Characteristics

| | CEPHIA | CHAVI | | | |
|---|---|---|---|---|---|
| | DP2 | 001 | Pitt Panel 2 | RV217 | Total |
| Longstanding | 10 | 24 | 48 | 0 | 82 |
| Recent | 0 | 26 | 39 | 17 | 82 |
| Total | 10 | 51 | 87 | 17 | 164 |

Equal numbers of recent (<9 months)/longstanding

Multi-clade (A, A/E, B, C); samples on ART

Tested downselected “best antigen” sets from Pitt Panel 2 analysis

Isotypes: IgA, IgG, IgG3, IgG4, IgM, pIgR

Binding and Avidity Measurements

Downselected Antigen Panel.

IgA:

gp41 ID epitope; p24

dIgA:

Consensus Group M Linear V3 epitope; gp41 ID epitope; p24

IgG:

Linear Clade B V3 epitope (binding and avidity); Scaffolded Clade B V3; Linear C1 epitope; Linear Clade B V1 epitope; Clade A, B gp140 (avidity); Consensus Clade A/E, B, C gp140 (avidity); p66 (avidity); Clade B gp140 (4 separate antigens, avidity); CRF01 A/E V2 linear epitope; Clade B V2 linear epitope; Clade A V2 linear epitope

IgM:

CRF07 (B/C) gp140 (binding and avidity); gp41 (binding and avidity); T/F Clade C gp140 (2 separate antigens); Consensus Group M gp140; p24

IgG3:

Consensus Clade C gp140 (avidity); gp41 (binding and avidity); Scaffolded Clade C V1V2 (avidity); p24 (binding and avidity); p31 (avidity); p66 (binding and avidity); T/F Clade C gp140 (2 separate antigens); Clade A gp140; Consensus Clade A gp140; Consensus Clade B gp140; Clade C gp140; Consensus Group M gp120; Consensus Group M gp140; CRF07 (B/C) scaffolded V1V2

IgG4:

T/F Clade C gp140 (2 separate antigens, binding and avidity); Consensus Group M gp120 (binding and avidity); Consensus Group M gp140; Clade B gp140 (3 separate antigens, binding and avidity); p24 (avidity); Consensus Clade A gp140 (avidity); Consensus Clade B gp140 (avidity)

Methods for Down-Selection Based on 9 Month Cut Off.

(1) Use each measurement as a continuous predictor of Classification in a logistic regression; (2) Use each measurement as a categorical predictor of Classification in a logistic regression; (3) Rank order measurements by the absolute value of the correlation with EDSC; (4) Rank order measurements by the mean difference between Classification; (5) Rank order measurements by the difference in positive response rate; (6) Combine the order score for methods 1-5; (7) Use the pairwise difference as a continuous predictor of Classification in a logistic regression.

Algorithm for Determining “Best” Measurements.

For Each of the First 6 Methods:

(a) Discriminant function analysis (DFA) for all possible sets of 3 to 5 measurements. (b) Misclassification error rate for the solution for all possible sets. (c) The lowest error rate for each method was determined by measurement combination and output the measurements in that solution

For difference scores a DFA was run on the top difference scores that had the lowest correlations.

The best combination of difference scores were then combined with the top model measurements using a stepwise approach.

Candidate Measurements (<9 Months Vs Longstanding Infection).

1. IgG3 Clade C gp140C

2. IgG4 avidity T/F Clade C gp140

3. IgG4 avidity Clade B gp140

4. Difference score: IgG Citrate gp41 ID epitope—IgG PBS gp41 ID epitope

TABLE 35
Classification: Recent vs. Longstanding

Assigned Group

| | | Longstanding | Recent | Total |
|---|---|---|---|---|
| Group | Longstanding | 63 | 3 | 66 |
| | Recent | 6 | 64 | 70 |
| | Total | 69 | 67 | 136 |

Recency error rate (t=270 days)=3/66=4.5%

Nominal FRR (T=2 years)=0/66=0%

Total error rate=9/136=6.6%

### Conclusions

Multiple subsets of HIV-1 specific antigen/antibody pairs that are excellent candidates for determination of recent (<9 months) vs longstanding infection. Additionally, a subset of 4 direct binding and antibody avidity measurements are able to distinguish recent (<9 months) vs longstanding infection in a globally relevant panel with a misclassification rate of 4.5%, including samples from patients treated with ARV.

Advantages of Incidence Assay.

The “best 4” set of analytes in all solutions encompasses multiple specific antibody isotypes and specificities.

Known markers of recency—IgG3 binding to gp140 Env

Maturation of immune response acute—>chronic infection—Increasing gp41 ID avidity; Increasing gp140 avidity in chronic infection

Known markers of chronic infection—IgG4 binding and avidity

Breadth of response using native and consensus sequences—Multi-clade Envs; Sequences from circulating strains vs. recombinant proteins; Recognition of unique antigenic features (conformational epitopes, T/F virus sequences)

Potential Improvements.

Time.

Selection of measurements was defined based on a 9 month cutoff for recent infection. Other selected cutoff times could yield a different array of measurements that may improve MDRI.

Recency Weighting.

The final analysis employs probability weighting that can be adjusted to tune the recency threshold (i.e. 40-50% cutoff).

Additional Measurements.

Additional envelope proteins (conformations), glyans, and peptides may improve FRR. Increasing dilution for IgG avidity measurements to increase recent/longstanding resolution.

Decreased Cost and Increased Scope.

Multi-Dimensional Array. New brilliant dyes enable multiplexing in additional dimension (collaborative development with VRC).

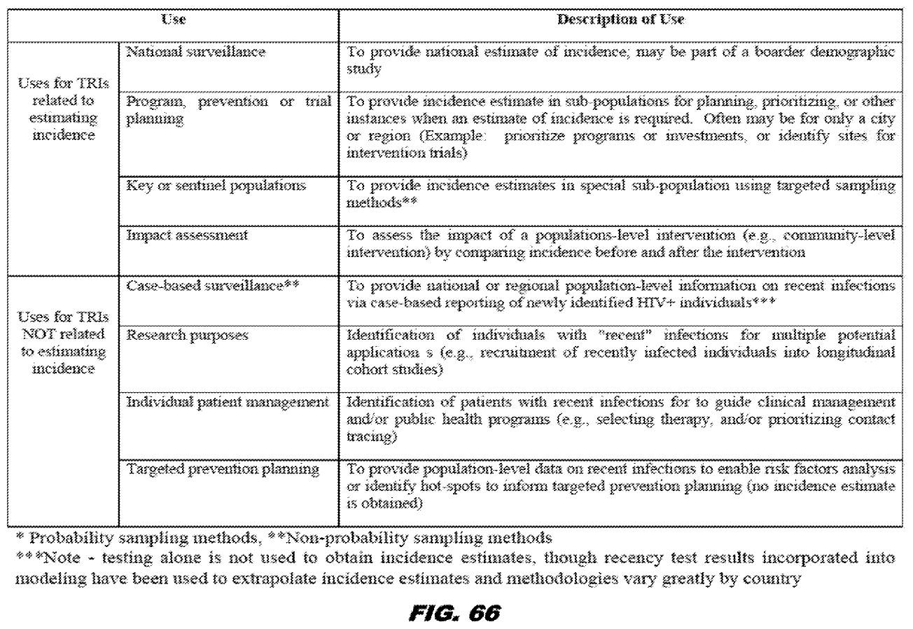

See FIG. 66 for Use Cases.

Experiment.

The inventors tested a blinded, prospectively collected evaluation panel from the CEPHIA Repository, consisting of 365 specimens spanning recent to longstanding infection. The panel was categorized into 3 subsets as follows. Subset 1 consisted of 131 specimens used for Mean Duration of Recent Infection (MDRI) calculation, ranging in time from 0-2 years from the Estimated Date of Detectable Infection (EDDI). Subset 2 consisted of 134 specimens greater than 2 years from the latest plausible date of detectable infection (LP-DDI), and subset 3 consisted of 58 challenge specimens on Antiretroviral Therapy (ART), greater than 2 years from LP-DDI.

Specimens were tested in a blinded manner using the 4 biomarkers selected from the downselection panel (BF1266 gp140 IgG3 binding, CH505 T/F gp140 IgG4 avidity magnitude, SC42261 gp140 IgG4 avidity magnitude, and the ratio of the log transformed gp41 ID IgG epitope binding and avidity magnitude). Results from this panel were evaluated using the classification equation to calculate the canonical value for each specimen. Additionally, a recent or longstanding probability score was calculated for each specimen. The classification output (recent or longstanding) were sent to FIND and to SACEMA for results un-blinding.

Samples were classified as recent or longstanding using results of the classification equation and additionally applying a posterior probability weighting criteria (i.e. recency probability cutoff @ 50%, 47.5%, 45%, 42.5% or 40%). After unblinding, the data were compared to the Limiting Antigen EIA (LAg) assay, which is the gold standard for incidence estimation in the field. Comparisons were made using the Duke Binding Antibody Multiplex Assay as a standalone test, in combination with viral load testing (VL=1000), separating ART treated samples vs. non-ART treated samples, and in Subtype B infection vs. non-Subtype B infection.

See FIG. 67. Specimens from patients on ART and VL<40 at time of specimen collection.

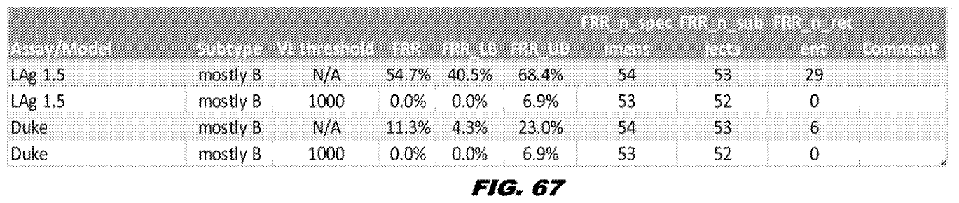

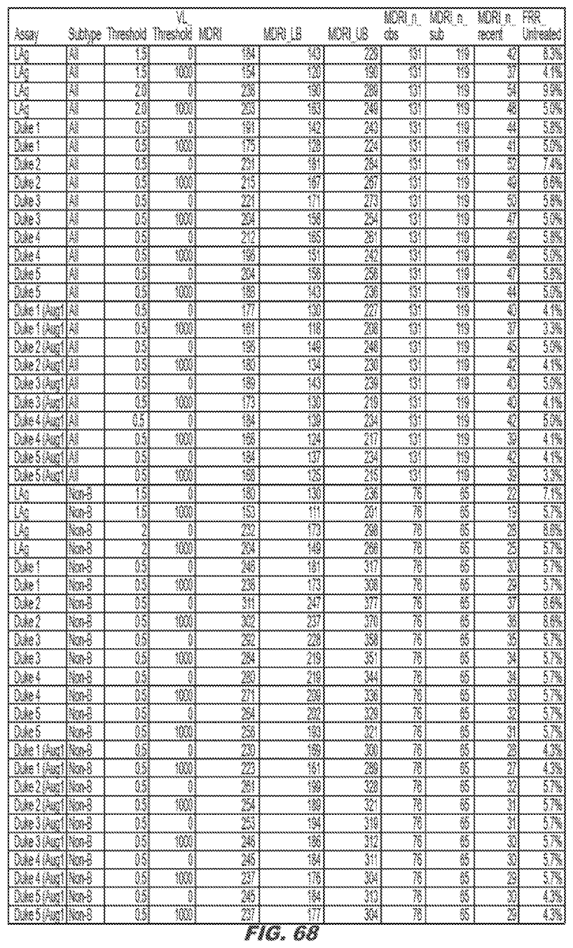

See FIG. 68. Patient Specimens from November.

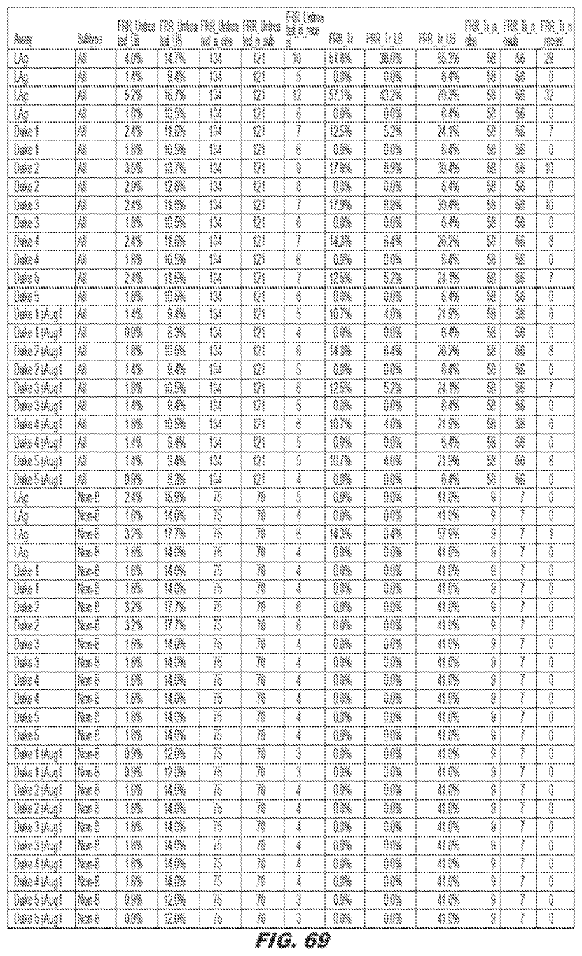

See FIG. 69. Patient Specimens from November.

### Example 9 Discriminant Function Analysis and Antibody Dynamics Distinguish HIV-1 Recent and Longstanding HIV-1 Infection

Abstract

Accurate HIV-1 incidence estimation is critical to the success of HIV-1 prevention strategies. Current assays are limited by high false recent rates (FRR) in certain populations, and a short mean duration of recent infection (MDRI). Dynamic early HIV-1 antibody (Ab) response kinetics were harnessed to identify a novel set of biomarker and combination of biomarkers for improved incidence assays. We conducted retrospective analyses on circulating antibodies from known recent and longstanding infections and evaluated binding and avidity measurements of Env and non-Env antigens (Ags) and multiple antibody forms (i.e. IgG, IgA, IgG3, IgG4, dIgA, and IgM) in a diverse panel of 164 HIV-1 infected participants (Clades A, B, C). Discriminant function analysis (DFA) identified an optimal set of biomarkers which were subsequently evaluated in a 365 specimen, blinded biomarker validation panel. These biomarkers included: IgG3/Clade C gp140, IgG4 avidity/transmitted/founder (T/F) Clade C gp140, IgG4 avidity/Clade B gp140, and IgG avidity/gp41 immunodominant regions (ID). MDRI was estimated at 215 (95% CI: 197-296) or alternatively, 267 days (215-320). FRR in untreated and treated subjects was 5.0% (1.8%-10.5%) and 3.6% (0.4%-12.3%), respectively. Thus, computational analysis of dynamic HIV-1 antibody isotype and antigen interactions during infection enabled design of a promising novel HIV-1 recency assay for improved cross-sectional incidence estimation.

### Introduction

Accurate estimates of HIV-1 incidence (i.e. the number of new HIV infections in a population in a defined period of time) are critical for planning and evaluating the success of HIV-1 prevention strategies (D1, D2). Recent advances in novel preventative measures, including vaccines (D3), treatment-as-prevention (D4) and Pre-Exposure Prophylaxis (PrEP) (D5), have changed the landscape of HIV-1 prevention. However, currently available cross-sectional HIV-1 incidence assays have limited utility in difficult to classify populations (ART treated subjects, elite controllers and subtype D infection (D6-D9)). This is often due to high false recent rate (FRR) (i.e. specimens infected for more than a recency time cut off time “T”, often chosen to be 2 years, that are classified as recently infected) (D6) and a mean duration of recent infection (MDRI) of approximately 4-5 months (D6, D10). Thus, the number of persons that must be surveyed in order to generate incidence estimates with a useful level of precision is unmanageably large in all but a small number of high incidence countries (D11, D12). Development of HIV-1 incidence assays using novel biomarkers must meet several key criteria for assay performance, including a longer MDRI and decreased FRR. The WHO/UNAIDS Incidence Assay Critical Path Working Group (D2, D13) recommends an ideal MDRI of between 6-12 months post infection with a FRR <2%. Achievement of these goals is critical to the accurate assessment of HIV-1 treatment and prevention efforts, the design of HIV-1 vaccine trials, and monitoring the epidemic in pursuit of an AIDS free generation.

We, and others, have previously characterized the sequential progression of HIV-1 antibody responses in acute through chronic infection and found that HIV-1 specific antibody isotypes and subclasses are extraordinarily dynamic during the early phase post acquisition and thus may be suitable for discriminating recent from longstanding infection (D14-D18). These include early markers of HIV infection, such as IgM, IgG and IgA antibodies to gp41 and Gag, which are among the earliest antibody specificities to arise post-infection (D14, D15). IgG antibodies to additional specificities within the HIV envelope are elicited sequentially, with a delay in anti-gp120 antibodies (D15). The earliest epitope specific responses appear first to the immunodominant region (ID) of gp41 and in the variable loop 3 (V3) region for gp120, then later in infection to the CD4 binding site and the membrane proximal external region (MPER) (D14, D15). Maturation of antibody responses includes an increase in antibody avidity for specific HIV-1 antigens, elevation of IgG4 responses, and may also include an increase in breadth of recognition of multiple HIV-1 subtypes during the transition from recent to longstanding infection (D15, D19). Notably, in the early phase of infection, some antibody responses exhibit a rapid increase in titer followed by declining kinetics. In particular, IgG3 antibody responses to p55 Gag, gp41 Env, and p66 RT decline in acute infection, in contrast to the same antigen specific IgG1 responses (D16, D17). Env IgA also demonstrates a marked decline both systemically and in the mucosa during recent infection (D14). Thus, we hypothesized that a multi-parameter approach that includes antibody isotypes and subclasses, presence or absence of certain epitope specificities, antibody avidity and recognition of unique HIV-1 envelope antigens would more accurately distinguish recent from longstanding infection.

Our approach builds upon this previous work profiling antibody kinetics from acute to chronic infection using multiplex binding measurements (D14-D16, D18, D20). Here, we determined epitope specificities and antibody isotypes and subclasses displaying distinct kinetic profiles through the course of HIV-1 infection for multiple HIV-1 subtypes. Multi-parameter measurements, presence or absence of a response along with the magnitude and avidity, were evaluated by machine learning with discriminant function analysis (DFA) to identify novel combinations of naturally occurring antibody biomarkers for cross-sectional HIV-1 incidence testing. In one embodiment, a set of four biomarkers for advancement toward an improved HIV-1 incidence test.

Results

Multiplex Incidence Assay Strategy

The humoral response to HIV-1 infection is marked by the dynamic appearance and disappearance of certain antibody isotypes and subclasses to different viral antigens from acute to longstanding infection. To harness this information for an HIV-1 incidence assay, we tested different antibody forms (IgM, IgG, IgG3, IgG4, IgA) in concert with a wide variety of HIV-1 antigens (peptides and proteins derived from env, gag, pol genes). The analysis includes the presence or absence of the response along with the magnitude and avidity of the antibody response when present. As part of the discovery phase, we utilized peptide microarray technology to identify linear sequences from the full gp160 envelope that could differentiate recent from longstanding infection. We selected an antigen set that would comprehensively cover the epitopes and antigen structures that would be most likely to be reactive with immune sera from recent to chronic infection, including transmitted founder envelope proteins. We evaluated plasma from 70 recent and 66 longstanding HIV-1 infections (“training panel”) of multiple subtypes (A, AE, B, C) from the CHAVI 001, RV217 cohorts and the CEPHIA repository, including 39 patients on antiretroviral therapy (Table 36). A recency cutoff of 9 months (270 days) was used to train the model for this analysis based upon initial characterization of candidate biomarkers using samples from the CEPHIA Repository and CHAVI001 acute infection cohorts. Estimated date of seroconversion (EDSC) was not known for all participants with longstanding infection; however, time since sample collection was >270 days since infection based on other parameters as described above.

TABLE 36
Subtype and recency classification of specimens in the training panel

| | | | ART | Time since EDSC |
|---|---|---|---|---|
| Classification | N | Clade | use (%, N) | (mean, range) |
| Recent | 12 | A1 | 0%, 0 | 100.6, [3, 225] |
| | 10 | B | 10%, 1 | 134.7, [33, 265] |
| | 16 | C | 0%, 0 | 149.5, [19, 261] |
| | 10 | CRF01_AE | 10%, 1 | 199.5, [146, 261] |
| | 22 | ND | 23%, 5 | 185.6, [32, 253] |
| Longstanding | 3 | A1 | 0%, 0 | 407.5, [286, 529] |
| | 40 | B | 53%, 21 | 1581.3, [283, 4673] |
| | 4 | C | 0%, 0 | 350.8, [335, 382] |
| | 19 | ND | 0%, 0 | 358.0, [358, 358] |

Recent infection was defined as <270 days from either the estimated date of seroconversion or any first positive HIV-1 test (if known) or the maximum time since infected based on Fiebig staging at enrollment (27). Samples were characterized as longstanding if >270 days from either the estimated date of seroconversion or from enrollment in an HIV-1 infection cohort. EDSC=Estimated Date of Seroconversion; ND=not determined; NA=not applicable. Information on subtype was provided by source cohorts where this was available.

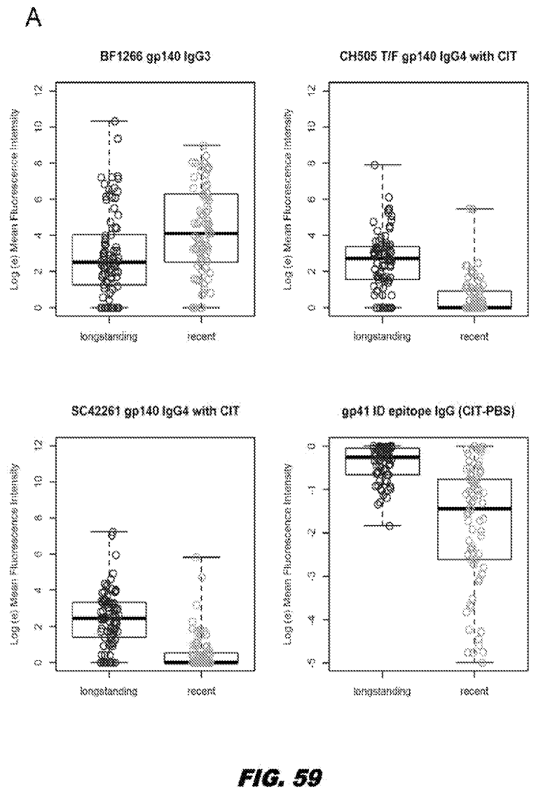

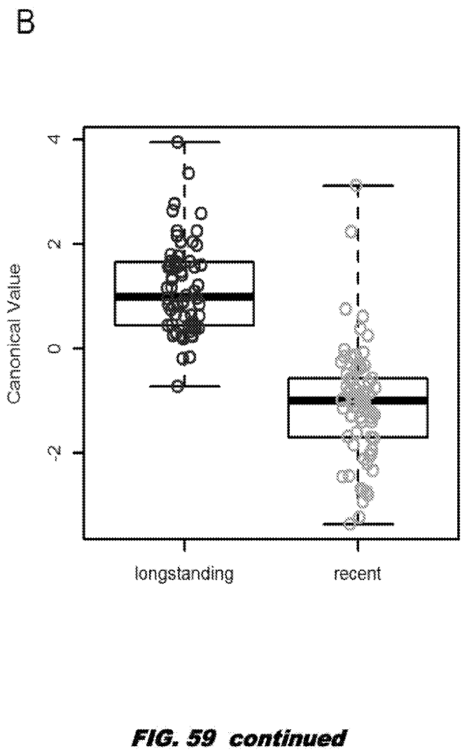

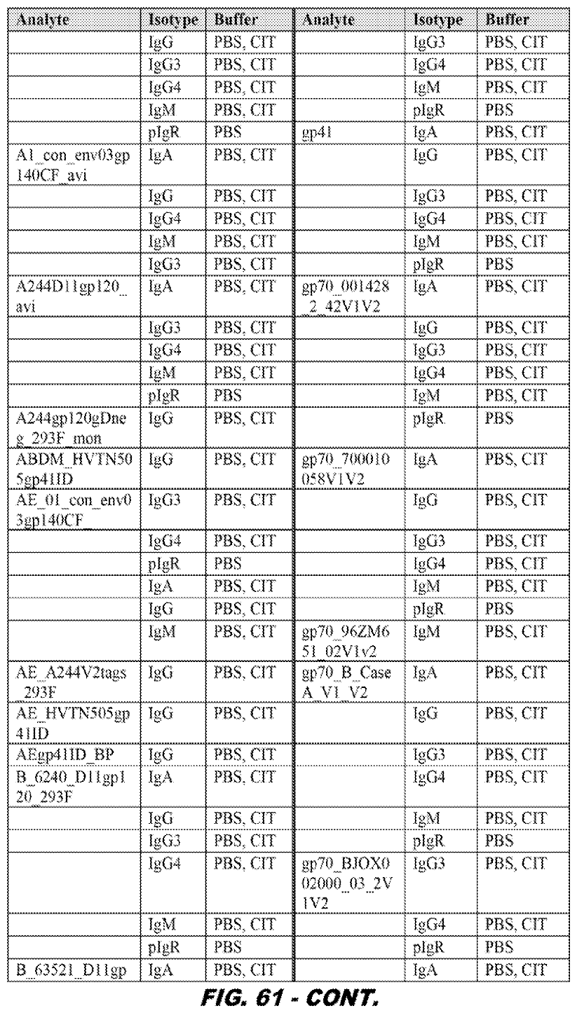

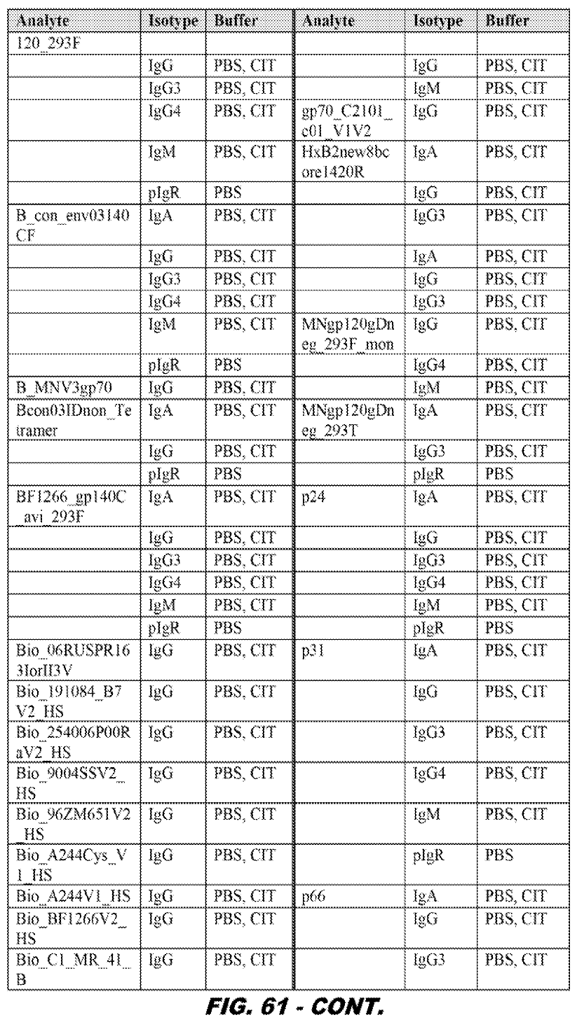

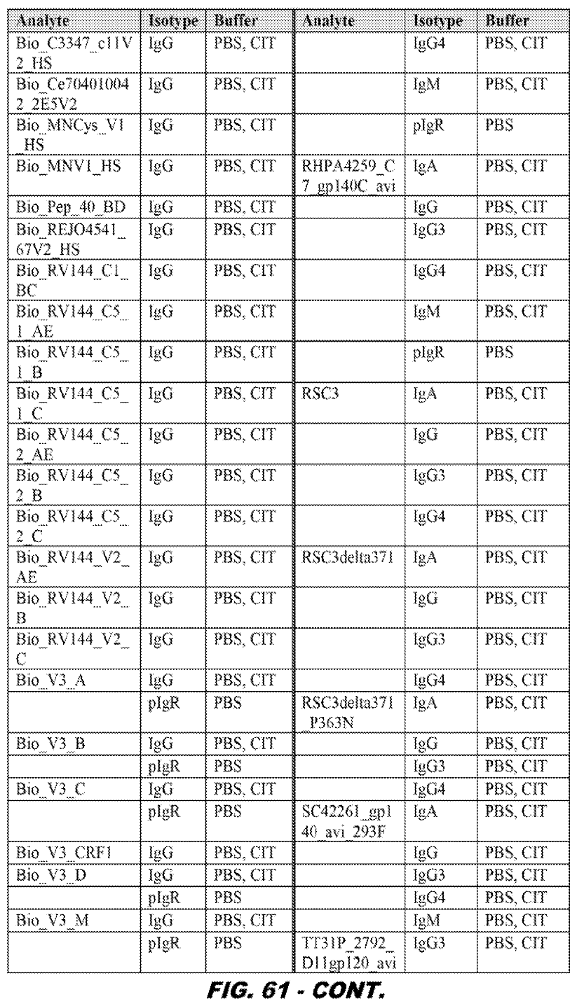

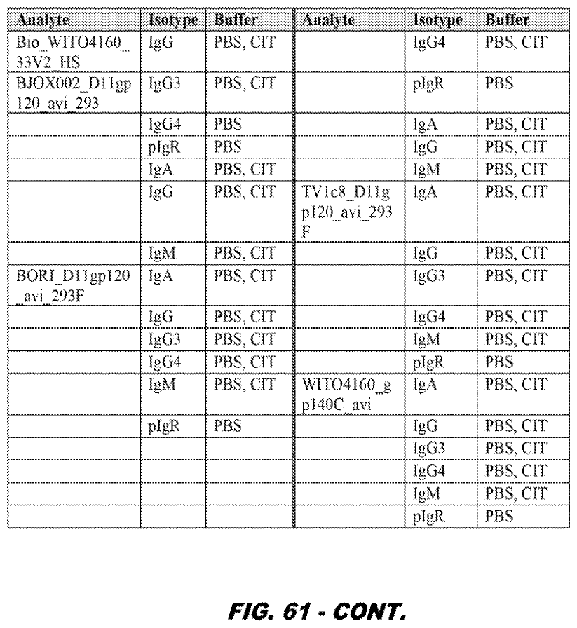

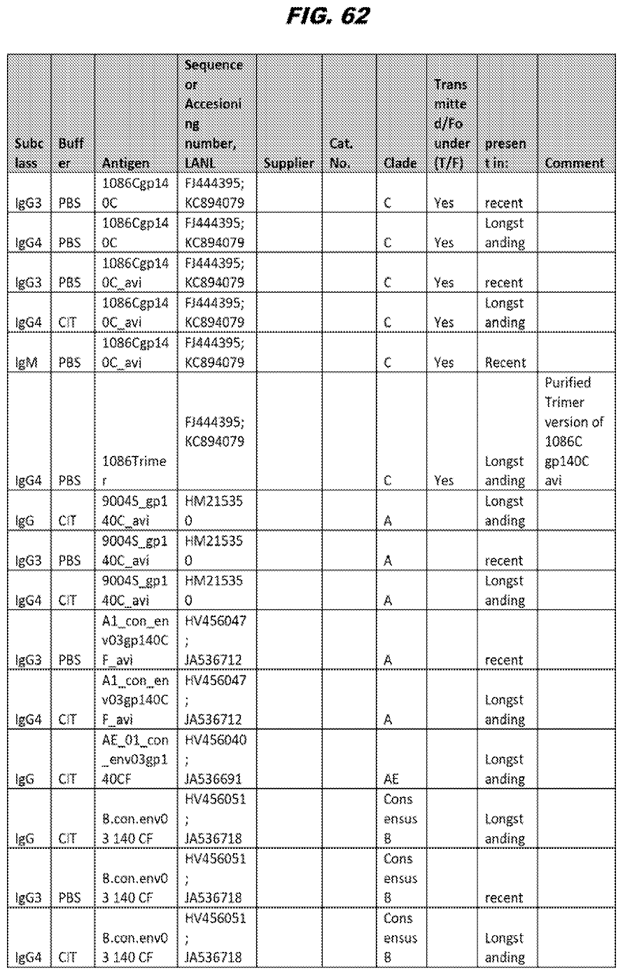

To determine the antibody/antigen measurements that most accurately categorize patient samples as recent or longstanding HIV infection, we performed discriminant function analysis (DFA) of all possible combinations of 3-6 Ab/Ag combinations from 505 possible Ab/Ag measurements (FIG. 61). DFA identified a set of four antibody/antigen biomarkers (FIG. 59) that classified recent versus longstanding infection with a 0% FRR (T=2 years) and a 4.4% overall misclassification rate, including samples on ART (Table 37). As supported by previous observations (D15), Env binding to IgG3 was more strongly associated with recent than longstanding infection (FIG. 59, Panel A). Consistent with a delayed elevation of IgG4 and antibody avidity in HIV-1 infection, Env IgG and IgG4 Env binding avidity were associated with longstanding infection (FIG. 59, Panel A). We found that specific antigens were most sensitive for discriminating these responses (i.e. SC42261 and CH505 T/F gp140 for IgG4 avidity and gp41 immunodominant epitope for IgG).

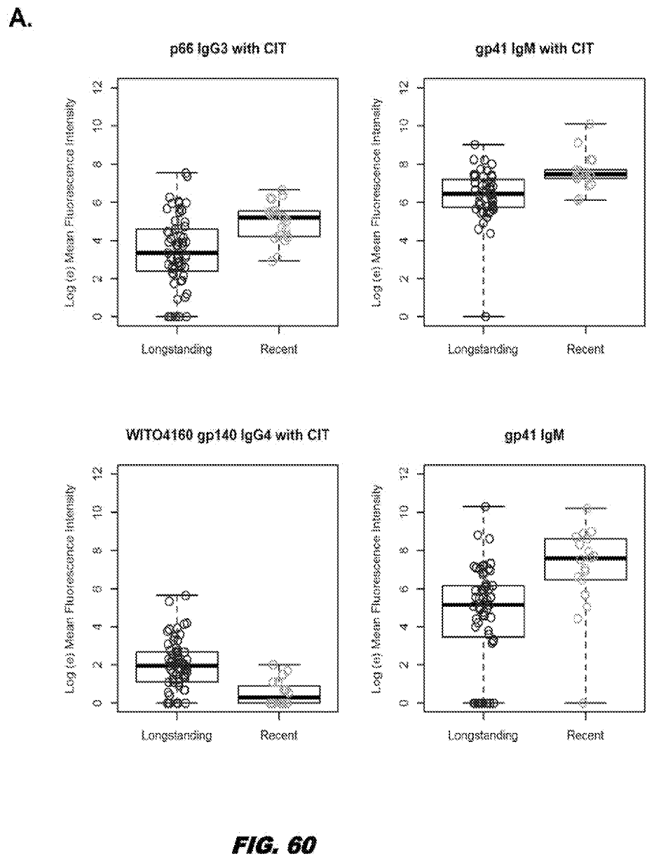

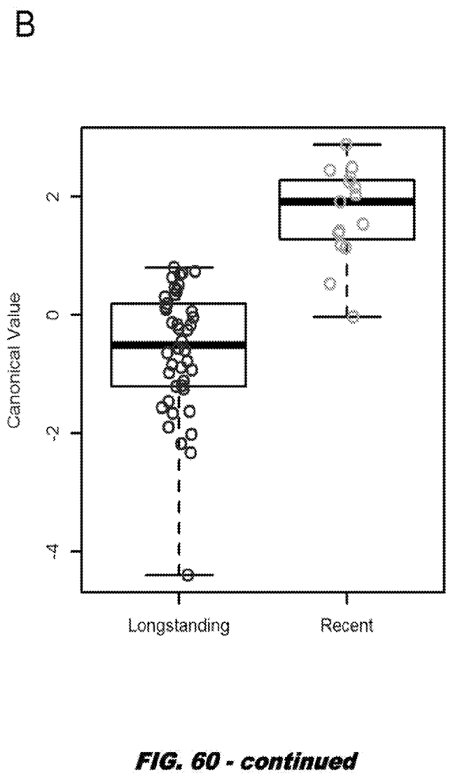

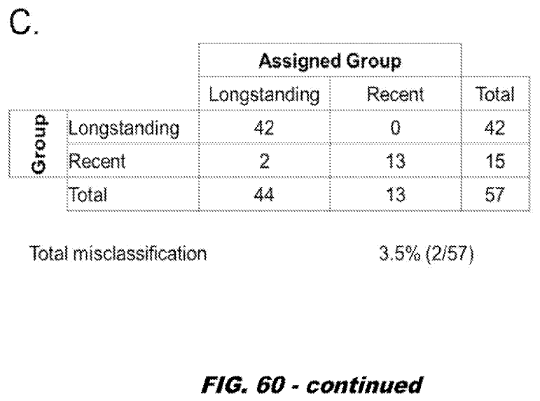

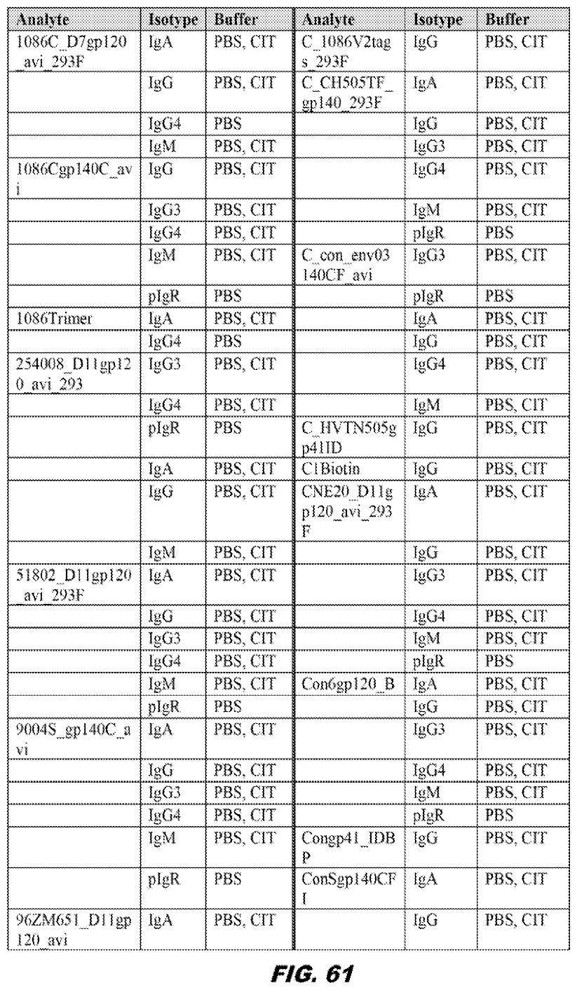

Responses to the four individual biomarkers exhibited overlap between recent and longstanding specimens; therefore discriminant function analysis was used to generate a canonical score based on all four measurements for each specimen (FIG. 59, Panel B). This set of four biomarkers achieved low overall misclassification rates as follows: 3/69 (4.5%) of longstanding specimens were misclassified as recent and 6/70 (8.6%) of recent specimens were misclassified as longstanding using a cutoff of 270 days. Total misclassification rate, including specimens from 39 patients on ART was 9/136 (6.6%). A false recent rate of 0% was achieved using a cutoff of T=2 years, with a total misclassification rate of 4.4% (Table 37). These 4 measurements were similar in type and epitope specificity to candidate biomarkers identified during the development phase (FIG. 60). This taken together with the low misclassification rate provided excellent rationale for further biomarker validation in a prospective, blinded validation panel.

TABLE 37
Classification of samples from Clades A, AE, B and C Cohorts yields
False Recent Rate = 0%.

| | | Assigned Group | | |
|---|---|---|---|---|
| | *T = 2 years | Longstanding | Recent | Total |
| Group | Longstanding | 66 | 0 | 66 |
| | Recent | 6 | 64 | 70 |
| | Total | 72 | 64 | 136 |
| | Total misclassification | | 4.4% (6/136) | |

Samples were tested via Binding Antibody Multiplex Assay (BAMA) and the magnitude of the response for each of the 4 best antibody-antigen combinations was analyzed using discriminant function analysis. Group=Classification based on days from Estimated Date of Seroconversion (EDSC) less than or greater than T=2 years. Assigned group=classification predicted by canonical values obtained via BAMA.

We next tested the four candidate biomarkers in a blinded validation panel. The panel comprised specimen sets for MDRI estimation (infected less than 800 days), FRR estimation (untreated, infected more than two years) and challenge specimens (treated, infected more than two years). All specimens used for MDRI and FRR estimation had viral loads available (Table 38).

TABLE 38
Composition and distribution of specimens in the validation panel

| | | | N | N | | |
|---|---|---|---|---|---|---|
| | | ART | specimens | specimens | N | N |
| Subset | Timing | treated | VL <= 100 | VL > 100 | specimens | subjects |
| MDRI | <800 days post- | No | 7 | 125 | 132 | 120 |
| | infection | | | | | |
| FRR | >2 yrs post- | No | 14 | 120 | 134 | 121 |
| | infection | | | | | |
| Challenge | >2 yrs post- | Yes | 56 | 2 | 58 | 56 |
| (ART) | infection | | | | | |

The panel comprised specimen sets for Mean Duration of Recent Infection (MDRI) estimation (infected less than 800 days), False Recent Rate (FRR) estimation (untreated, infected more than two years) and challenge specimens (treated, infected more than two years). All specimens used for MDRI and FRR estimation had viral loads available.

Resulting MDRIs and FRRs were estimated for a range of BAMA classifications (at varying PP thresholds) and compared to the performance of a commercially available incidence assay, the Sedia HIV-1 Limiting Antigen (LAg)-Avidity enzyme immunoassay, which was applied to the same panel at the Blood Systems Research Institute (San Francisco, Calif.). Both sets of results were additionally combined with a supplementary viral load threshold (specimens classified as recent by the assay but with viral load below 100 copies/ml re-classified as longstanding).

TABLE 39
Mean Duration of Recent Infection and False Recent Rate from
blinded validation panel

| Assay/Model | VL threshold | MDRI (CI), days | FRR untreated (CI) | FRR ART treated (CI) |
|---|---|---|---|---|
| BAMA (PP >= 0.5) | 0* | 215 (167-266) | 5.0% (1.8%-10.5%) | 3.6% (0.4%-12.3%) |
| BAMA (PP >= 0.4) | 0* | 267 (215-320) | 5.8% (2.4%-11.6%) | 10.7% (4.0%-21.9%) |
| LAg (ODn < 1.5) | 0* | 157 (117-202) | 8.3% (4.0%-14.7%) | 51.8% (38.0%-65.3%) |
| LAg (ODn < 2.0) | 0* | 210 (163-261) | 9.9% (5.2%-16.7%) | 57.1% (43.2%-70.3%) |
| BAMA (PP >= 0.5) | 100 | 199 (152-250) | 4.1% (1.4%-9.4%) | 0.0% (0.0%-6.4%) |
| BAMA (PP >= 0.4) | 100 | 251 (199-305) | 5.0% (1.8%-10.5%) | 0.0% (0.0%-6.4%) |
| LAg (ODn < 1.5) | 100 | 138 (102-178) | 4.1% (1.4%-9.4%) | 0.0% (0.0%-6.4%) |
| LAg (ODn < 2.0) | 100 | 187 (143-235) | 5.0% (1.8%-10.5%) | 0.0% (0.0%-6.4%) |

*No viral load threshold, but analysis restricted to specimens for which viral load is available

Results for Mean Duration of Recent Infection (MDRI) and False Recent Rate (FRR) for Binding Antibody Multiplex Assay (BAMA) and Limiting Antigen (LAg) assays using the blinded CEPHIA Proof-of-Concept Panel are shown. PP=posterior probability. ODn=normalized optical density. CI=95% confidence interval.

Results are summarized in Table 39. The BAMA assay had an MDRI of 215 days (95% CI: 167-266) at the standard posterior probability threshold compared to 157 days (117-202) for LAg (at standard ODn threshold of 1.5) on the same specimen set. The FRR in untreated specimens was 5.0% (1.8%-10.5%) and in the ART-treated (challenge) subset was 3.6% (0.4%-12.3%), as compared to 8.3% (4.0%-14.7%) and 51.8% (38.0%-65.3%) respectively for LAg. When combined with a viral load threshold, MDRI was reduced to 199 days (152-250), but the FRR in untreated specimens was reduced to 4.1% (1.4%-9.4%) and in treated specimens to 0.0% (0.0%-6.4%). LAg (ODn<1.5) in combination with viral load produced an MDRI of 138 days (102-178) and identical FRRs. At the alternative PP cutoff of 0.40, the MDRI without viral load was 267 days (215-320) and when combined with viral load was 251 days (199-305).

The MDRI for the BAMA assay (at standard PP cutoff) was longer than that of LAg (at standard ODn threshold) both without and with the use of viral load (59 days, p=0.060 and 61 days, p=0.040, respectively), with very similar FRRs on untreated specimens. Without the use of supplemental viral load, the FRR on treated subjects was significantly better than that of LAg. Using the alternative PP cutoff of 0.40, the MDRI for the BAMA assay was substantially and significantly longer than that of LAg at the standard ODn threshold: 110 days, p<0.001. This demonstrates potential for the combination of binding and avidity measurements of gp41 ID IgG, Env IgG3, and Env IgG4 to improve cross-sectional incidence estimation.

### Discussion

Large scale investments in HIV prevention and treatment efforts and accurate assessment of the effectiveness of these programs requires robust population-level incidence estimation approaches.

The complexity of early pathogenesis, including diversity in antibody responses among individuals and HIV subtypes, regional genetic diversity in virus, HIV disease state and ART use has limited progress in novel assay development. In particular, leading candidate assays all appear to require explicit viral load testing to reduce otherwise unacceptably high FRRs among treated individuals and elite controllers, for both of which populations with mature epidemics are increasingly enriched.

Improvements in HIV-1 incidence estimation can be achieved through combining multiple tests into a Multi-Assay Algorithm (MAA), including antibody measurements, p24 antigen, viral diversity and/or viral load determination. Recency Incidence Testing Algorithms (RITA) including Serologic Testing Algorithm for Recent HIV Seroconversion (STARHS) (21-24), have also been used in combination to refine estimates of incidence, with varying degrees of success (D6, D21). However, utility of these assays may be limited due to increased costs per sample, sample volume required, and complexity of the assay. To make usefully precise incidence estimates available from feasible sample sizes, recent infection tests require a delicate balance between sufficiently long MDRI and sufficiently low FRR (D6). A recent advance in ELISA based assays is the commercially available Limiting Antigen (LAg) avidity assay (D25) that utilizes avidity to a multi-clade recombinant gp41 ID epitope (rIDR-m) and achieves a low FRR of 1.3% when elite controllers and patients on ART treatment are excluded (D6). Results of an independent analysis of five standard incidence assays, including LAg, (BioRad avidity, Vitros, LS-Vitros, LAg and BED) indicated a high FRR of 12.9-48.4% in elite controllers and 50.0-76.1% in ART treated individuals, indicating the need for novel biomarkers of HIV-1 incidence.

Considerations for development of HIV-1 incidence biomarkers must include expansion of the range of biomarkers to achieve greater separation between recent versus longstanding specimens. Current assays are limited in Env sequence, clade diversity, and antibody isotypes and rely on non-native protein sequences for assessment of binding antibody responses. A majority of currently available incidence assays measure avidity to a limited number of sequences, including varying recombinant forms of gp41 IgG (i.e. LAg, BED), multiple fusion proteins comprised of varying combinations of gp120/gp41, p24 and p36 (i.e. Vitros and LS-Vitros) and gp160 and p24 recombinant proteins derived from HIV-1, gp36 from HIV-2 and a synthetic polypeptide mimicking an artificial HIV-1 group 0 specific epitope (i.e. Bio-Rad avidity). Recent assays have begun to include binding and avidity to other Env and non-Env sequences such as p66 and gp120 (D26), which display a range of maturation kinetics from recent to longstanding infection. However, additional biomarkers such as clade diversity and maturation kinetics of antibody isotypes and subclasses (e.g. increasing antibody avidity from recent to chronic infection) are likely to provide the greatest increase in MDRI and greatest reductions in FRR. This included binding and avidity to a range of linear epitopes, diverse Env and non-Env proteins (native and consensus sequences) and Ig isotypes and subclasses. This work expanded the set of biomarkers by building upon previous observations that IgG subclasses and Env specificities display differential kinetics during the course of recent infection (D15, D16). The panel of antigens was built to balance potential antibody biomarkers of recency (gp41 epitopes, dIgA, IgG3, IgM) with markers of longstanding infection (gp120 epitopes, IgG4, avidity to gp41) and to diversify the antigen panel through addition of globally relevant, circulating strains (Clades A and C, linear epitopes from Clades A, B, C, D, Group M Consensus) and transmitted founder Env proteins. The resulting antigen panel confirmed the use of traditional incidence biomarkers such as gp41 avidity index to discriminate recent vs. longstanding infection, while bypassing the limitations of current assays through the use of additional recency (IgG3) and longstanding (avidity to IgG4) biomarkers. Additionally, the top solution included markers of clade and sequence diversity (T/F envelope, Clades B and C gp140 proteins). While the gp41 ID region is a key HIV-1 recency marker and may be responsible for some of the activity to the gp140 proteins, the relative contribution of the gp41 ID epitope to the gp140 proteins is unknown and likely only one of the drivers of this activity. Other regions in gp140 (i.e. V2 and V3 antigens) also scored highly in development phase, though were not part of the “best” antigen set. It is thus likely that the gp140 reactivity is driven by multiple epitope specificities, including gp41 ID, V3 and possible conformational epitopes.

Short of constructing detailed hypothetical surveillance scenarios, it is possible to discern superiority of one test over another if it has a longer MDRI or lower FRR (or both) while being non inferior on the other (if not both). By this assessment, the newly reported BAMA platform produces at least one model of recent infection which is statistically superior to the Sedia LAg assay, which was constructed on a well-characterized unblinded ‘training panel’ (365 specimens; Clades B and C infection), using a time-based definition of recent infection as a gold standard. In particular, an advantage of the newly developed biomarker algorithm is the strongly improved FRR achieved on specimens from participants on ART, without the need for a supplemental viral load analysis. Data generated in the optimization, training and model development phases are also well suited for additional in silico optimization to further improve MDRI and FRR as required for product development. These improvements may include alternate epitope specificities that scored consistently scored highly in the optimization phase (i.e. linear V3 epitopes, alternate gp140 sequences), but were not part of the “best 4” antigen panel. Additionally, these data will provide key insight for future development of the assay to include testing samples from Subtype D infection and samples from participants treated early with ART.

Use of HIV-1 incidence tests with an MDRI between 240-280 days will enable applications such as national surveillance, determination of intervention effectiveness, and estimation of incidence in key populations that are currently not feasible due to large sample size requirements. Use of improved assays with longer MDRI are estimated to result in a cost savings between $10-24 million USD per year due to the reduction in sample size (D12). Thus, the biomarkers of HIV-1 recency reported here are excellent candidates to advance for further development of a novel HIV-1 incidence algorithm.

Methods

Incidence assay performance is defined by a tradeoff between MDRI and FRR which minimizes the variance in incidence estimates. In order to access robust learning algorithms, models for recent-infection case definitions were built by minimizing classification error on a recency status assignment on a “training panel” of specimens, with recent infection defined as less than 9 months post EDSC. Resulting classification models, or threshold based modifications, were then validated by estimating MDRI and FRR from blinded testing of a non-overlapping “biomarker validation panel”. Laboratory processes and training analyses were completely blinded to the clinical background data on validation panel specimens.

Specimen Panels

The training specimen panel comprised plasma or serum from 66 recent (9 months or less from EDSC to specimen collection) and 70 longstanding (more than 9 months since EDSC) HIV-1 infections of multiple subtypes (A, AE, B, C) (Table 36). Sources included HIV-1 acute and chronic cohorts from the Center for HIV/AIDS Vaccine Immunology (CHAVI) (CHAVI 001, CHAVI 008), United States Military HIV Research Program (USMHRP) (RV217), and from the specimen repository created and maintained by the Consortium for the Evaluation and Performance of HIV-1 Incidence Assays (CEPHIA) which has been previously described (D6, D11). EDSC was determined as described previously (D6). In some cases where an EDSC could not be calculated, a duration of infection was estimated based upon the Fiebig stage documented at cohort enrollment, using the maximum cumulative duration of infection as described by Cohen et al. (27).

Biomarker validation was performed by testing and classifying specimens from the CEPHIA Proof-of-Concept (PoC) biomarker validation panel and estimating MDRI and FRR. The panel comprised 132 well-characterized untreated specimens drawn within 800 days of EDSC (120 unique subjects) for MDRI estimation, 134 specimens (121 subjects) from untreated and 58 specimens (56 subjects) from treated longstanding infection for FRR estimation. Specimens were largely from subjects infected with subtypes B (56%) and C (43%). Additionally a number of reproducibility controls were included. Composition of the panel is summarized in Table 38.

Binding Antibody Multiplex Assay (BAMA)

We profiled HIV-1 specific binding antibody responses in participant serum or plasma as described previously (D14-D16). We evaluated antibody binding to a unique and broad set of HIV-1 antigens that included gp120, gp140, and V1-V2 antigens of multiple subtypes. This panel includes a downselected set of envelope antigens for assessing cross-clade breadth of binding responses. Additional antigens included multiple clade specific and consensus linear peptides for the gp41 immunodominant region, V1, V2 V3, C1 and C5 epitopes, p31 integrase (Jena Bioscience), p66 RT (Protein Sciences), p24 Gag (BD Biosciences). Serum or plasma were diluted in BAMA assay diluent (1% milk-blotto, 5% normal goat serum, 0.05% Tween-20) and incubated with antigen coupled microspheres. Samples were incubated with either anti-human IgG-PE (Southern Biotech; catalog number 9040-09), oranti-human IgA (Jackson Immunoresearch; catalog number 109-065-011), anti-human IgG3 (Calbiochem; catalog number 411483), anti-human IgG4 (BD Pharmingen; catalog number 555879), or anti-human IgM (Southern Biotech; 2020-08) followed by Streptavidin-PE (BD Biosciences) and detection on either a Bioplex 200 (BioRad, Hercules, Calif.) or Luminex FM3D machine (Luminex Corp, Austin, Tex.). All samples were depleted of IgG using a MultiTrap system (GE Biosciences) per manufacturer's instruction, prior to assessment of IgA or IgM specific antibodies. Dimeric IgA was detected using a recombinant polymeric-Ig receptor (pIgR, (D28)). Samples were incubated with pIgR overnight in assay diluent (1% BSA, 0.05% Tween-20), followed by incubation with microsphere beads covalently coupled to the antigen of interest. pIgR was detected using anti-human pIgR (secretory component, SC) detection followed by anti-mouse IgG-PE (Southern Biotech, catalog number 1030-09). Controls for IgG assays included titrated HIVIG (NIH AIDS Reagent Program), 7B2 IgG (D29), CH58 IgG (D30), and CH22 IgG mAb (D31). IgA specific controls included: titrated 7B2 monomeric IgA. Controls for the pIgR assay included: 7B2 monomeric IgA, 7B2 SIgA, 7B2 dIgA (D32), and purified SC coupled beads. Controls for IgM assays included titrated 2F5 (D33) and CLL246 (gp41 specific) IgM (D34). Controls for IgG3 and IgG4 assays included a titrated purified human IgG3 or IgG4 standard curve, and purified IgG3 or IgG4 coupled beads. Negative controls in each assay included Normal Human Serum (NHS, Sigma Aldrich) and blank beads. Each experiment was performed using good clinical laboratory practice (GCLP)-compliant conditions, including tracking of positive controls by Levey-Jennings charts.

Antibody Avidity

Assessment of antibody avidity was determined by BAMA with the following modifications. After formation of antigen antibody immune complexes, a 15 minute dissociation step (Na-Citrate, pH 3.0, Teknova) (D25) was included prior to addition of secondary detection antibody. Retained binding magnitude (mean fluorescence intensity or MFI) in the presence of dissociation buffer was used as a measurement of antibody avidity in the statistical models.

Statistics

Discriminant Function Analysis

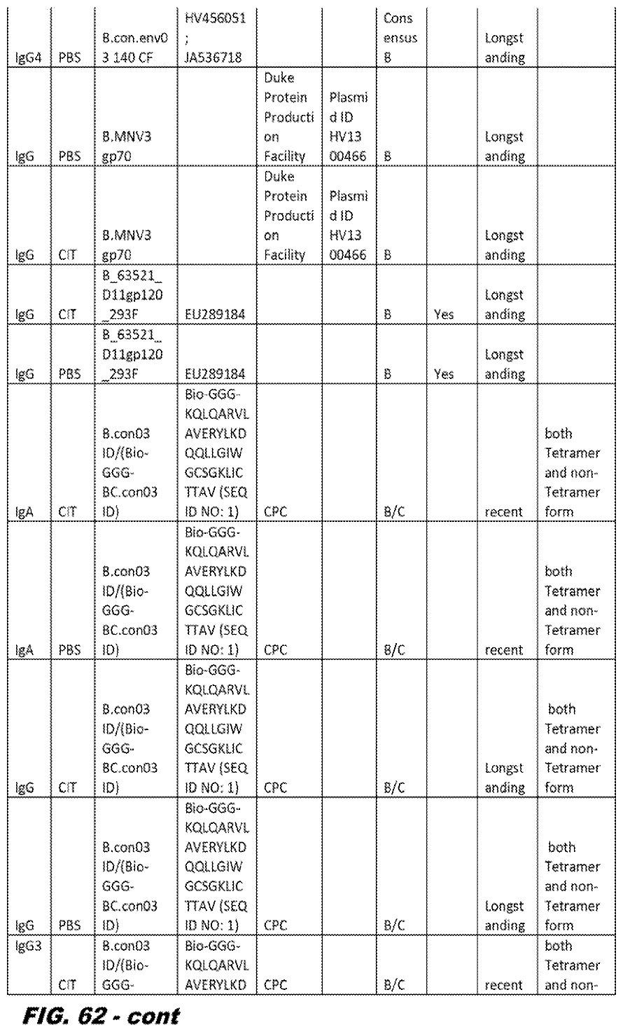

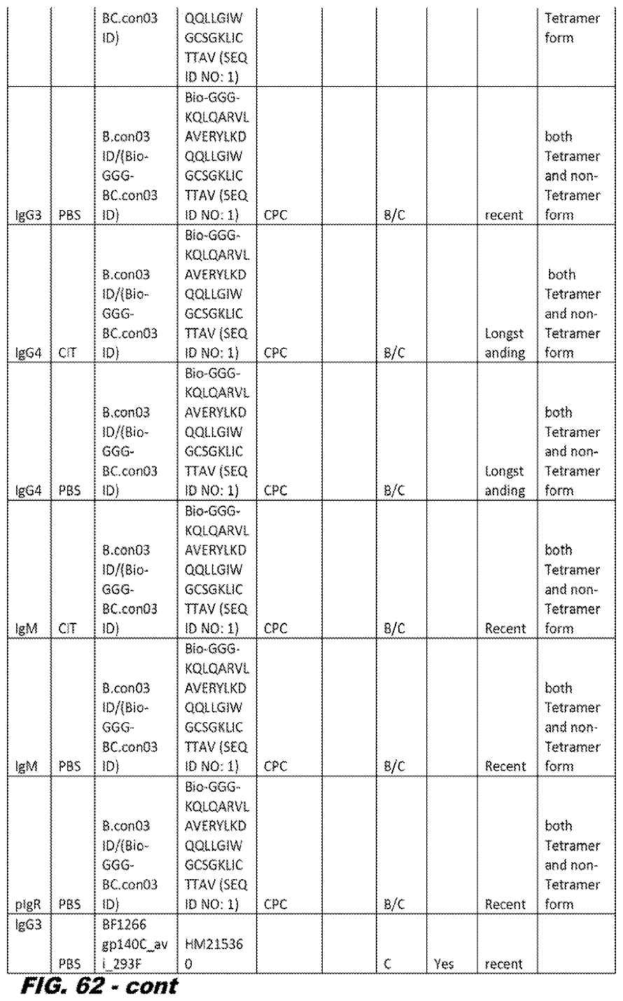

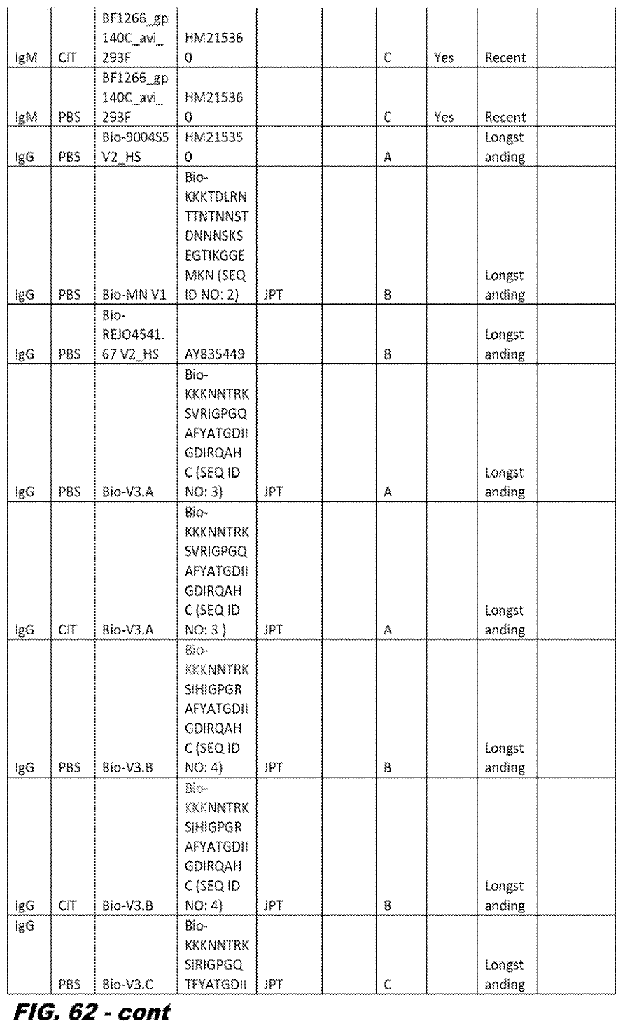

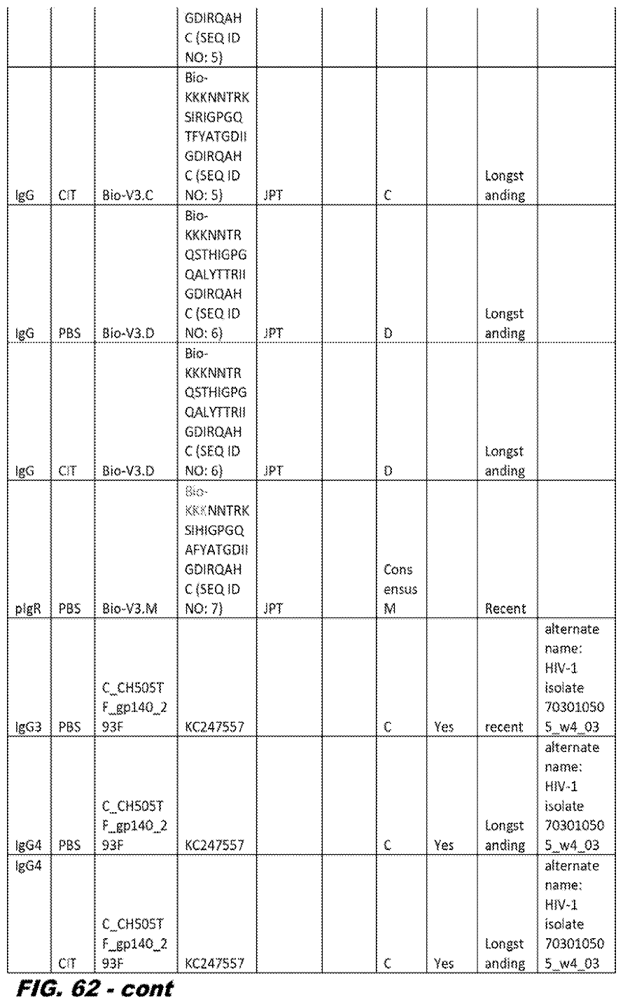

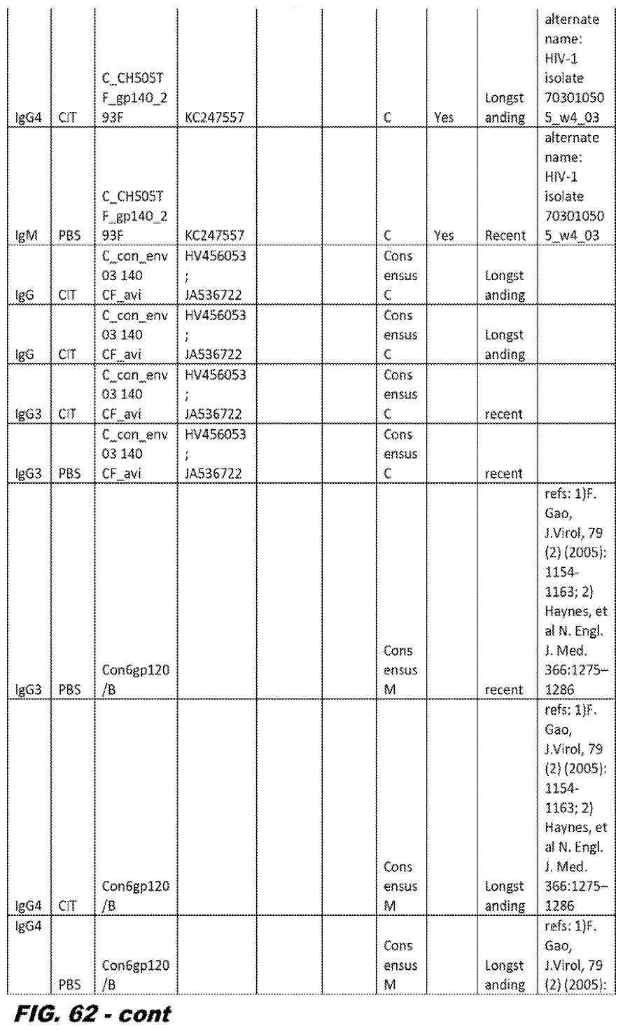

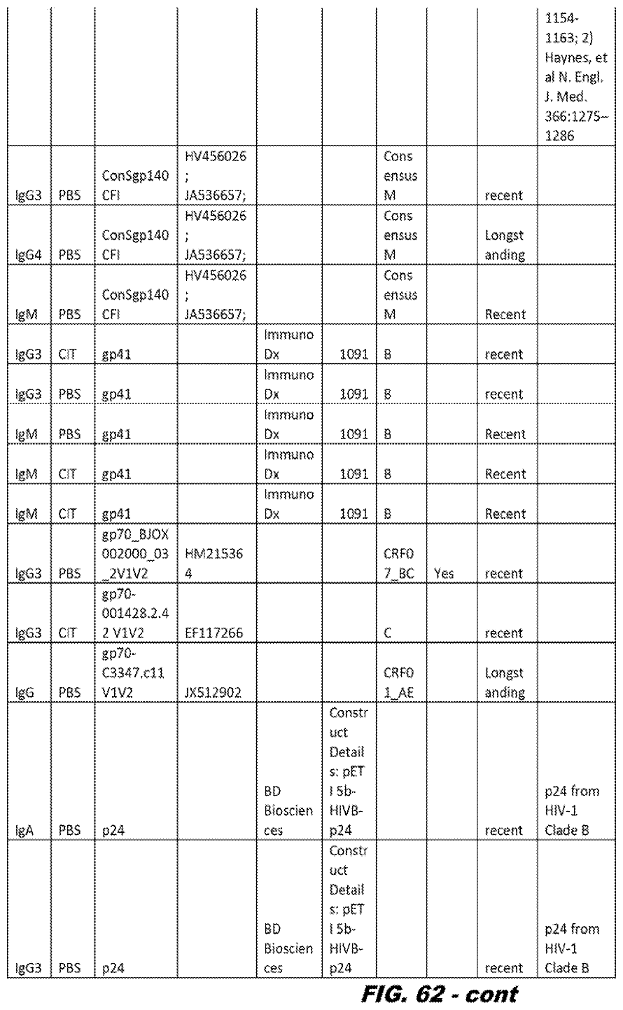

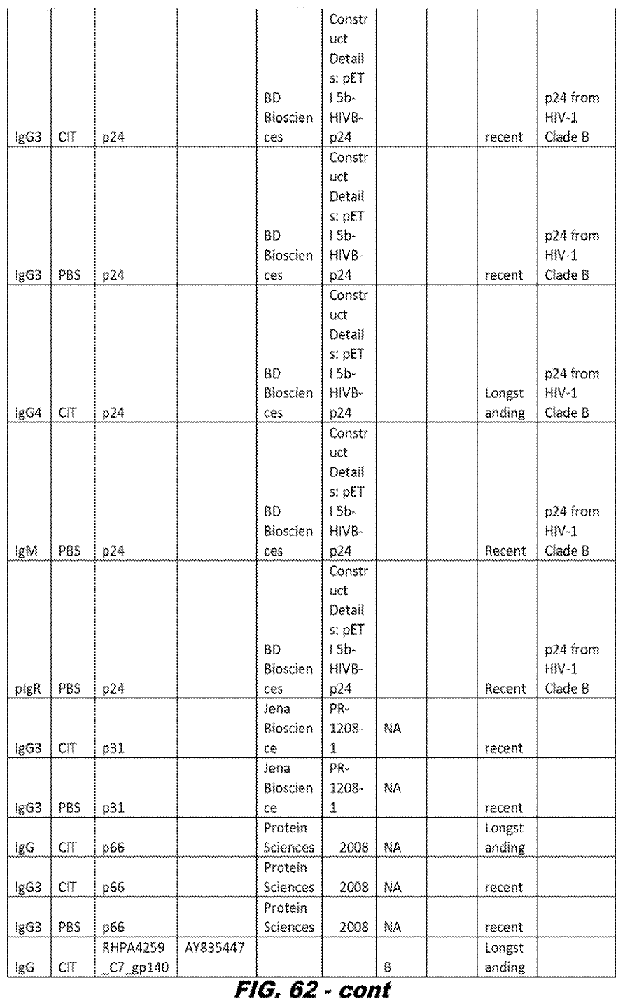

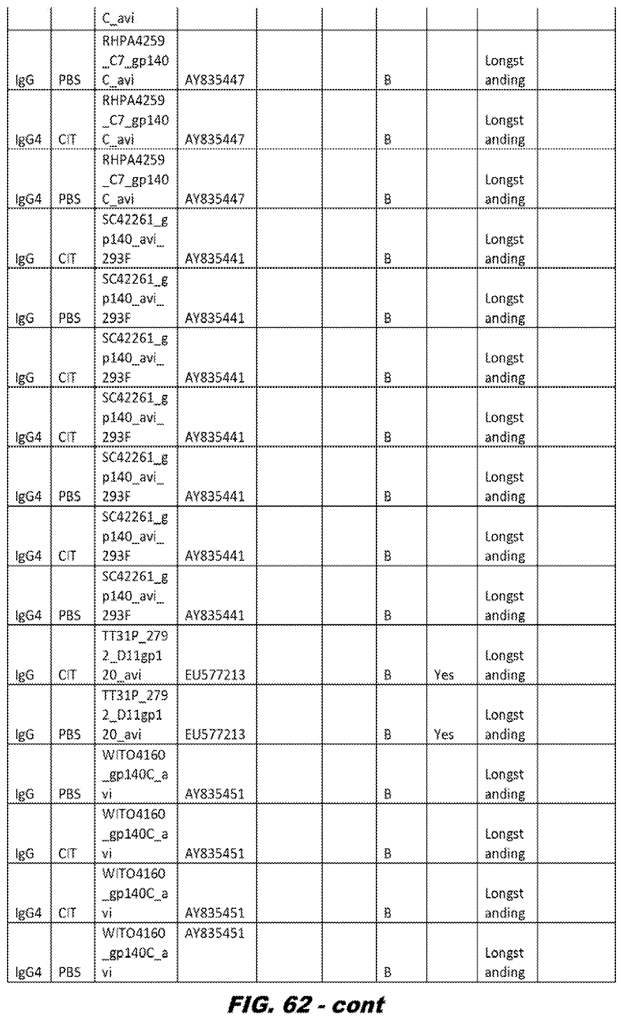

To down select from 505 antigen/antibody measurements, we used six criteria to rank order the measurements: (1) logₑ MFI as a continuous predictor of HIV status (recent vs. longstanding) use to rank order the measurements by the Wald χ² for the coefficient for each measurement as a predictor of group classification (recent/longstanding); (2) MFI as a categorical variable for positive/negative response (based on a cutoff of 100 MFI) as a categorical predictor of HIV status, then by the Wald χ² for that parameter; (3) rank order the measurements by the absolute value of Pearson correlation coefficient of each measurement with time since EDSC; (4) rank order the measurements by the mean difference between HIV status groups; (5) rank order measurements by the difference in positive response rate from the positivity cutoff of 100 MFI between HIV status groups; (6) compute the pairwise difference between all pairs of measures for each patient to use a continuous predictor of HIV status group and rank order by Wald χ² for that parameter. The top twenty measurements according to each criterion were then selected. Twenty was the number selected to make the final sample space manageable while minimizing the risk of excluding a measurement with good discriminatory power. Among the 120 candidate measurements collected from the six ranking strategies, 12 were represented more than once, leaving 108 unique measurements. FIG. 62 shows a listing a subset of Ab/Ag biomarkers that could be used in alternative embodiments of biomarkers X1-X4 in FIG. 60. A skilled artisan appreciates that in some embodiments: any of the Clade C gp140 envelopes listed in FIG. 62 could be used to obtain measurement X1; any one of T/F Clade C gp140 envelope could be used to obtain measurement X2; any one of Clade B gp140 envelopes could be used to obtain measurement X3; any one of gp41 ID could be used to obtain measurement X4.

To determine a set of measurements that had the optimum predictive power we applied a discriminant function analysis model (D35) to all possible combinations of 3,4,5, and 6 measurements from the 108 measurement down selected list. We recorded the misclassification error rate (recent patients categorized as longstanding and longstanding patients categorized as recent) for each possible unique combination. With sets of four measurements, a total error rate of 6.6% was reached that was not further reduced by including additional measurements.

Sample classification: The discriminant function analysis (DFA) determines classification of the subjects using Canonical correlation 1. There are also regression parameters for each measurement so that a discriminant value (DV) score can be calculated. DV score is calculated for both classes, Recent and Longstanding. Whichever score is higher is how the subject is classified. DVj is used interchangeably with Cj, wherein j is the category recent (_{R}) or (_{L}).

Using discriminant function analysis, classification coefficients (“c”) for each Ab/Ag biomarker measurement can be extracted—see Table 40 and Table 41 for non-limiting embodiments of classification coefficients for a set of four measurements. Measurements of these Ab/Ag biomarkers, for e.g. but not limited in previously unclassified samples, are fed through the classification equation. For a four measurement set the equation is:

Cⱼ=c₀ⱼ+cⱼ₁X₁+cⱼ₂X₂+cⱼ₃X₃+cⱼ₄X₄,

where there is an intercept and a coefficient for each measurement for each group j, here recent or longstanding. If C_{Recent}>C_{Longstanding} then the new subject is classified as recent and vice versa. Additionally, the posterior probability (PP) criteria used for assigning group membership can improve the accuracy of the classification in use cases where the desired definition of recent is >9 months. While usually a value of PP(recent)>=0.5 is used as the criterion for assigning a categorization of recent to an observed case, other thresholds can be entertained, yielding variations on the model which offer different tradeoff between MDRI and FRR—previously noted to be the ultimate determinants of test performance. The equation described above is used to calculate the probability of a sample being classified as recent or longstanding (C_{recent} and C_{longstanding}). In some embodiments, Posterior probabilities (PP) are used to determine thresholds for recent or longstanding classification, and can be adjusted to fine tune MDRI and FRR. For example, a PP=0.5 indicates a threshold where a sample with a probability C_{recent}>0.5 would be classified as recent. A PP=0.4 indicates a threshold where a sample with probability C_{recent}>0.4 would be classified as recent. In general, lowering the PP threshold for recency lengthens the MDRI.

TABLE 40
shows combined panel discriminant coefficients (See FIG. 59).

| | Constant | IgG3_PBS_BF1266_gp140C_avi_293F | IgG4_CIT_C_CH505TF_gp140_293F |
|---|---|---|---|
| Classification | c₀ | c₁ | c₂ |
| Longstanding | −3.81 | 0.49 | 0.69 |
| Recent | −5.35 | 1.03 | 0.12 |

| | | IgG_CIT_Bcon03IDNon_Tetramer- |
|---|---|---|
| | IgG4_CIT_SC42261_gp140_avi_293F | IgG_PBS_Bcon03IDNon_Tetramer |
| Classification | c₃ | c₄ |
| Longstanding | 0.98 | −1.26 |
| Recent | 0.40 | −2.55 |

Without being bound by theory, the combined panel discriminant coefficients are expected to be the same or very similar for similar Ab/Ag pairs from FIG. 61.

TABLE 41
shows Pitt panel discrimination coefficients (See FIG. 60)

| | Constant | IgG3_CIT_P66 | IgG4_CIT_WITO4160_gp140C_avi | IgM_CIT_gp41 | IgM_PBS_gp41 |
|---|---|---|---|---|---|
| Classification | c₀ | c₁ | c₂ | c₃ | c₄ |
| Longstanding | −24.44 | 0.45 | 0.92 | 7.43 | −0.61 |
| Recent | −34.27 | 1.13 | −0.38 | 8.23 | −0.26 |

Without being bound by theory, the Pitt panel discriminant coefficients are expected to be the same or very similar for similar Ab/Ag pairs from FIG. 61.

Estimation of MDRI and FRR

MDRI was estimated by binomial regression of probability of obtaining an assay-recent result as a function of time since seroconversion, and integrating this probability from seroconversion to the recency cut-off time T, using approaches described previously (6) which have been implemented in the R package inctools (Welte A, Grebe E, McIntosh A, Bäumler P. inctools: Incidence Estimation Tools [R package]. 2016. The model for P_{R}(t) was fit using all specimens drawn <=800 days post-EDSC, but to obtain MDRI, the integral is only evaluated from 0 to T. MDRI=∫₀^{T}P_{R}(t), T=2 years. Available from: https://cran.r-project.org/web/packages/inctools/). Confidence intervals were obtained by resampling subjects in 10,000 bootstrap iterations.

To evaluate MDRI differences between recency tests, the variances of difference estimates were approximated using 10,000 subject-level bootstrapping iterations. P-values for differences were obtained from a two-tailed Z-test, with values less than 0.05 considered significant.

The FRR estimates were obtained by simply estimating the binomial proportion of patients testing recent at times more than T post seroconversion.

Study Approvals

Samples from participants in all research cohorts were collected following informed consent. Retrospective analysis was performed with approval from the Duke Medicine Institutional Review Board (IRB) for Clinical Investigations, Durham, N.C. (Protocol: Pro00039677).

### Example 10 Binding Antibody Multiplex Assay and Avidity Index Assay (BAMA/BAMA-AI) for HIV-1 Incidence Determination

Prepared Reagents:
Antigen conjugated beads: prepared according to Duke CFAR-GAP SOP CFAR03-0040.
Wash buffer: Bovine Serum Albumin (5 g); NaN3 (0.25 g); 250 μl Tween-20; 1 Packet Pierce PBS Packet Product #28374; all dissolved in 500 mL H₂O.
Assay diluent: (1% MILK BLOTTO (W/V), PHOSPHATE BUFFERED SALINE (PBS), 5% NORMAL GOAT SERUM, 0.05% TWEEN-20)

Procedure:

1. Thaw antigen-conjugated beads on ice. After thawing, vortex for at least 30 seconds (minimize exposure to light).

2. Prepare working bead mixture in wash buffer (current optimized bead count: 5000 beads per analyte per well).

3. Wet filter plate with 100 μl wash buffer and aspirate wash buffer.

4. Vortex bead mixture and add 50 μl/well in filter plate. Place on rotator and keep plate protected from light while not in use.

5. Vortex, then centrifuge plasma, serum, saliva and unfiltered mucosal samples in an aerosol safe rotor at 4500×g for 5 minutes.

6. Prepare samples and controls in assay diluent plate. Samples will be diluted in assay diluent (1% milk blotto (w/v), Phosphate buffered saline, 5% normal goat serum, 0.05% Tween-20) per optimized dilution factors. Current dilution factors for plasma/serum: IgG-PE: 1:10,000; IgG3: 1:40 or 1:100; IgG4: 1:40.

7. Aspirate bead mixture buffer using vacuum manifold.

8. Transfer 25 μl patient and control samples to appropriate wells in filter plate. Add assay diluent to blank wells. Protect the plate from light and incubate on rotator for 30-35 minutes (on high for the first 30 seconds).

9. Dilute detection antibody and detection reagent (if necessary) in assay diluent. Note: Detection antibody and detection reagents may be prepared at any point during the assay day before use. For anti-human IgG-PE, dilute to 2 μg/ml in assay diluent. For all other detection antibodies; dilute to 4 μg/ml in assay diluent.

10. Aspirate and wash plates 3 times with 100 μl/well of the wash buffer.

11. Add 100 ul PBS or 100 ul Na-Citrate buffer, pH 3.0 (Teknova) to appropriate sample wells. Protect the plate from light and incubate on rotator for 15 minutes (on high for the first 30 seconds).

12. Aspirate and wash plates 3 times with 100 μl/well of the wash buffer.

13. Add 100 μl of diluted detection antibody to beads in filter plate. Protect the plate from light and incubate on rotator for 30-35 minutes (on high for the first 30 seconds).

14. Aspirate and wash plates 3 times with 100 μl/well of the wash buffer.

NOTE: At this time, if detection antibodies used were PE conjugates, proceed to step 18. If antibodies were Biotin conjugates, follow steps 15-17.

15. Add 100 μl of diluted detection reagent to beads in filter plate. Protect the plate from light and incubate on rotator for 30-35 minutes (on high for the first 30 seconds).

16. Aspirate and wash plates 3 times with 100 μl/well of the wash buffer.

17. Resuspend beads in 100 μl/well wash buffer. Place on rotator to thoroughly resuspend beads. Run on Luminex or Bioplex system.

### LIST OF REFERENCES

A1. Alam, S. M., R. M. Scearce, R. J. Parks, K. Plonk, S. G. Plonk, L. L. Sutherland, M. K. Gorny, S. Zolla-Pazner, S. Vanleeuwen, M. A. Moody, S. M. Xia, D. C. Montefiori, G. D. Tomaras, K. J. Weinhold, S. A. Karim, C. B. Hicks, H. X. Liao, J. Robinson, G. M. Shaw, and B. F. Haynes. 2008. Human immunodeficiency virus type 1 gp41 antibodies that mask membrane proximal region epitopes: antibody binding kinetics, induction, and potential for regulation in acute infection. J Virol 82:115-125.
A2. Barnighausen, T., T. A. McWalter, Z. Rosner, M. L. Newell, and A. Welte. 2010. HIV incidence estimation using the BED capture enzyme immunoassay: systematic review and sensitivity analysis. Epidemiology 21:685-697.
A3. Bonsignori, M., M. A. Moody, R. J. Parks, T. M. Holl, G. Kelsoe, C. B. Hicks, N. Vandergrift, G. D. Tomaras, and B. F. Haynes. 2009. HIV-1 envelope induces memory B cell responses that correlate with plasma antibody levels after envelope gp120 protein vaccination or HIV-1 infection. J Immunol 183:2708-2717.
A4. Brenchley, J. M., D. A. Price, T. W. Schacker, T. E. Asher, G. Silvestri, S. Rao, Z. Kazzaz, E. Bornstein, O. Lambotte, D. Altmann, B. R. Blazar, B. Rodriguez, L. Teixeira-Johnson, A. Landay, J. N. Martin, F. M. Hecht, L. J. Picker, M. M. Lederman, S. G. Deeks, and D. C. Douek. 2006. Microbial translocation is a cause of systemic immune activation in chronic HIV infection. Nature medicine 12:1365-1371.
A5. Fiebig, E. W., C. M. Heldebrant, R. I. Smith, A. J. Conrad, E. L. Delwart, and M. P. Busch. 2005. Intermittent low-level viremia in very early primary HIV-1 infection. J Acquir Immune Defic Syndr 39:133-137.
A6. Friedman, J., S. M. Alam, X. Shen, S. M. Xia, S. Stewart, K. Anasti, J. Pollara, G. G. Fouda, G. Yang, G. Kelsoe, G. Ferrari, G. D. Tomaras, B. F. Haynes, H. X. Liao, M. A. Moody, and S. R. Permar. 2012. Isolation of HIV-1-neutralizing mucosal monoclonal antibodies from human colostrum. PloS one 7:e37648.
A7. Ganesan, A., P. K. Chattopadhyay, T. M. Brodie, J. Qin, W. Gu, J. R. Mascola, N. L. Michael, D. A. Follmann, and M. Roederer. 2010. Immunologic and virologic events in early HIV infection predict subsequent rate of progression. J Infect Dis 201:272-284.
A8. Hargrove, J. W., J. H. Humphrey, K. Mutasa, B. S. Parekh, J. S. McDougal, R. Ntozini, H. Chidawanyika, L. H. Moulton, B. Ward, K. Nathoo, P. J. Iliff, and E. Kopp. 2008. Improved HIV-1 incidence estimates using the BED capture enzyme immunoassay. AIDS 22:511-518.
A9. Haynes, B. F., P. B. Gilbert, M. J. McElrath, S. Zolla-Pazner, G. D. Tomaras, S. M. Alam, D. T. Evans, D. C. Montefiori, C. Karnasuta, R. Sutthent, H. X. Liao, A. L. DeVico, G. K. Lewis, C. Williams, A. Pinter, Y. Fong, H. Janes, A. DeCamp, Y. Huang, M. Rao, E. Billings, N. Karasavvas, M. L. Robb, V. Ngauy, M. S. de Souza, R. Paris, G. Ferrari, R. T. Bailer, K. A. Soderberg, C. Andrews, P. W. Berman, N. Frahm, S. C. De Rosa, M. D. Alpert, N. L. Yates, X. Shen, R. A. Koup, P. Pitisuttithum, J. Kaewkungwal, S. Nitayaphan, S. Rerks-Ngarm, N. L. Michael, and J. H. Kim. 2012. Immune-correlates analysis of an HIV-1 vaccine efficacy trial. The New England journal of medicine 366:1275-1286.
A10. Hodinka, R. L., T. Nagashunmugam, and D. Malamud. 1998. Detection of human immunodeficiency virus antibodies in oral fluids. Clin Diagn Lab Immunol 5:419-426.
A11. Janssen, R. S., G. A. Satten, S. L. Stramer, B. D. Rawal, T. R. O'Brien, B. J. Weiblen, F. M. Hecht, N. Jack, F. R. Cleghorn, J. O. Kahn, M. A. Chesney, and M. P. Busch. 1998. New testing strategy to detect early HIV-1 infection for use in incidence estimates and for clinical and prevention purposes. JAMA 280:42-48.
A12. Karita, E., M. Price, E. Hunter, E. Chomba, S. Allen, L. Fei, A. Kamali, E. J. Sanders, O. Anzala, M. Katende, and N. Ketter. 2007. Investigating the utility of the HIV-1 BED capture enzyme immunoassay using cross-sectional and longitudinal seroconverter specimens from Africa. AIDS 21:403-408.
A13. Kshatriya, R., A. A. Cachafeiro, R. J. Kerr, J. A. Nelson, and S. A. Fiscus. 2008. Comparison of two rapid human immunodeficiency virus (HIV) assays, Determine HIV-1/2 and OraQuick Advance Rapid HIV-1/2, for detection of recent HIV seroconversion. Journal of clinical microbiology 46:3482-3483.
A14. Levesque, M. C., M. A. Moody, K. K. Hwang, D. J. Marshall, J. F. Whitesides, J. D. Amos, T. C. Gurley, S. Allgood, B. B. Haynes, N. A. Vandergrift, S. Plonk, D. C. Parker, M. S. Cohen, G. D. Tomaras, P. A. Goepfert, G. M. Shaw, J. E. Schmitz, J. J. Eron, N. J. Shaheen, C. B. Hicks, H. X. Liao, M. Markowitz, G. Kelsoe, D. M. Margolis, and B. F. Haynes. 2009. Polyclonal B cell differentiation and loss of gastrointestinal tract germinal centers in the earliest stages of HIV-1 infection. PLoS medicine 6:e1000107.
A15. McMichael, A. J., P. Borrow, G. D. Tomaras, N. Goonetilleke, and B. F. Haynes. 2010. The immune response during acute HIV-1 infection: clues for vaccine development. Nat Rev Immunol 10:11-23.
A16. Nawa, M., T. Takasaki, M. Ito, S. Inoue, K. Morita, and I. Kurane. 2005. Immunoglobulin A antibody responses in dengue patients: a useful marker for serodiagnosis of dengue virus infection. Clin Diagn Lab Immunol 12:1235-1237.
A17. Pandori, M. W., J. Hackett, Jr., B. Louie, A. Vallari, T. Dowling, S. Liska, and J. D. Klausner. 2009. Assessment of the ability of a fourth-generation immunoassay for human immunodeficiency virus (HIV) antibody and p24 antigen to detect both acute and recent HIV infections in a high-risk setting. J Clin Microbiol 47:2639-2642.
A18. Parekh, B. S., M. S. Kennedy, T. Dobbs, C. P. Pau, R. Byers, T. Green, D. J. Hu, S. Vanichseni, N. L. Young, K. Choopanya, T. D. Mastro, and J. S. McDougal. 2002. Quantitative detection of increasing HIV type 1 antibodies after seroconversion: a simple assay for detecting recent HIV infection and estimating incidence. AIDS research and human retroviruses 18:295-307.
A19. Sakarovitch, C., F. Rouet, G. Murphy, A. K. Minga, A. Alioum, F. Dabis, D. Costagliola, R. Salamon, J. V. Parry, and F. Barin. 2007. Do tests devised to detect recent HIV-1 infection provide reliable estimates of incidence in Africa? J Acquir Immune Defic Syndr 45:115-122.
A20. Tomaras, G. D., J. M. Binley, E. S. Gray, E. T. Crooks, K. Osawa, P. L. Moore, N. Tumba, T. Tong, X. Shen, N. L. Yates, J. Decker, C. K. Wibmer, F. Gao, S. M. Alam, P. Easterbrook, S. Abdool Karim, G. Kamanga, J. A. Crump, M. Cohen, G. M. Shaw, J. R. Mascola, B. F. Haynes, D. C. Montefiori, and L. Morris. 2011. Polyclonal B cell responses to conserved neutralization epitopes in a subset of HIV-1-infected individuals. J Virol 85:11502-11519.
A21. Tomaras, G. D., and B. F. Haynes. 2009. HIV-1-specific antibody responses during acute and chronic HIV-1 infection. Curr Opin HIV AIDS 4:373-379.
A22. Tomaras, G. D., N. L. Yates, P. Liu, L. Qin, G. G. Fouda, L. L. Chavez, A. C. Decamp, R. J. Parks, V. C. Ashley, J. T. Lucas, M. Cohen, J. Eron, C. B. Hicks, H. X. Liao, S. G. Self, G. Landucci, D. N. Forthal, K. J. Weinhold, B. F. Keele, B. H. Hahn, M. L. Greenberg, L. Morris, S. S. Karim, W. A. Blattner, D. C. Montefiori, G. M. Shaw, A. S. Perelson, and B. F. Haynes. 2008. Initial B-cell responses to transmitted human immunodeficiency virus type 1: virion-binding immunoglobulin M (IgM) and IgG antibodies followed by plasma anti-gp41 antibodies with ineffective control of initial viremia. J Virol 82:12449-12463.
A23. Wei, X., X. Liu, T. Dobbs, D. Kuehl, J. N. Nkengasong, D. J. Hu, and B. S. Parekh. 2010. Development of two avidity-based assays to detect recent HIV type 1 seroconversion using a multisubtype gp41 recombinant protein. AIDS research and human retroviruses 26:61-71.
A24. Wilson, K. M., E. I. Johnson, H. A. Croom, K. M. Richards, L. Doughty, P. H. Cunningham, B. E. Kemp, B. M. Branson, and E. M. Dax. 2004. Incidence immunoassay for distinguishing recent from established HIV-1 infection in therapy-naive populations. Aids 18:2253-2259.
A25. Yates, N. L., J. T. Lucas, T. L. Nolen, N. A. Vandergrift, K. A. Soderberg, K. E. Seaton, T. N. Denny, B. F. Haynes, M. S. Cohen, and G. D. Tomaras. 2011. Multiple HIV-1-specific IgG3 responses decline during acute HIV-1: implications for detection of incident HIV infection. AIDS 25:2089-2097.
B1. Tomaras G D, et al. J Virol. 2008 December; 82(24):12449-63
B2. Yates, et al. AIDS. 2011 Nov. 13; 25(17):2089-97.
B3. Liao, et al. Immunity. 2013 Jan. 24; 38(1):176-86
B4 Yates, et al. Sci Transl Med 2014 Mar. 19; 6(228):228ra39
C1. Sharma, U. K., et al., Workshop summary: Novel biomarkers for HIV incidence assay development. AIDS Res Hum Retroviruses, 2012. 28(6): p. 532-9.
C2. Incidence Assay Critical Path Working, G., More and better information to tackle HIV epidemics: towards improved HIV incidence assays. PLoS Med, 2011. 8(6): p. e1001045.
C3. Rerks-Ngarm, S., et al., Vaccination with ALVAC and AIDSVAX to prevent HIV-1 infection in Thailand. N Engl J Med, 2009. 361(23): p. 2209-20.
C4. Cohen, M. S., et al., Prevention of HIV-1 infection with early antiretroviral therapy. N Engl J Med, 2011. 365(6): p. 493-505.
C5. Baeten, J. M., et al., Antiretroviral prophylaxis for HIV prevention in heterosexual men and women. N Engl J Med, 2012. 367(5): p. 399-410.
C6. Kassanjee, R., et al., Independent assessment of candidate HIV incidence assays on specimens in the CEPHIA repository. AIDS, 2014. 28(16): p. 2439-49.
C7. Yates, N. L., et al., HIV-1 gp41 envelope IgA is frequently elicited after transmission but has an initial short response half-life. Mucosal Immunol, 2013. 6(4): p. 692-703.
C8. Tomaras, G. D., et al., Initial B-cell responses to transmitted human immunodeficiency virus type 1: virion-binding immunoglobulin M (IgM) and IgG antibodies followed by plasma anti-gp41 antibodies with ineffective control of initial viremia. J Virol, 2008. 82(24): p. 12449-63.
C9. Yates, N. L., et al., Multiple HIV-1-specific IgG3 responses decline during acute HIV-1: implications for detection of incident HIV infection. AIDS, 2011. 25(17): p. 2089-97.
C10. Wilson, K. M., et al., Incidence immunoassay for distinguishing recent from established HIV-1 infection in therapy-naive populations. AIDS, 2004. 18(17): p. 2253-9.
C11. Curtis, K. A., M. S. Kennedy, and S. M. Owen, Longitudinal analysis of HIV-1-specific antibody responses. AIDS Res Hum Retroviruses, 2014. 30(11): p. 1099-105.
C12. Curtis, K. A., et al., Development and characterization of a bead-based, multiplex assay for estimation of recent HIV type 1 infection. AIDS Res Hum Retroviruses, 2012. 28(2): p. 188-97.
C13. Cohen, M. S., et al., The detection of acute HIV infection. J Infect Dis, 2010. 202 Suppl 2: p. S270-7.
C14. Santra, S., et al., Human Non-neutralizing HIV-1 Envelope Monoclonal Antibodies Limit the Number of Founder Viruses during SHIV Mucosal Infection in Rhesus Macaques. PLoS Pathog, 2015. 11(8): p. e1005042.
C 15. Liao, H. X., et al., Vaccine induction of antibodies against a structurally heterogeneous site of immune pressure within HIV-1 envelope protein variable regions 1 and 2. Immunity, 2013. 38(1): p. 176-86.
C16. Montefiori, D. C., et al., Magnitude and breadth of the neutralizing antibody response in the RV144 and Vax003 HIV-1 vaccine efficacy trials. J Infect Dis, 2012. 206(3): p. 431-41
C17. Duong, Y. T., et al., Detection of recent HIV-1 infection using a new limiting-antigen avidity assay: potential for HIV-1 incidence estimates and avidity maturation studies. PLoS One, 2012. 7(3): p. e33328.
C18. Montefiori, D. C., Measuring HIV neutralization in a luciferase reporter gene assay. Methods Mol Biol, 2009. 485: p. 395-405.
C19. Mastro, T. D., et al., Estimating HIV Incidence in Populations Using Tests for Recent Infection: Issues, Challenges and the Way Forward. J HIV AIDS Surveill Epidemiol, 2010. 2(1): p. 1-14.
C20. Curtis, K. A., et al., Evaluation of a multiplex assay for estimation of HIV-1 incidence. PLoS One, 2013. 8(5): p. e64201.
C21. Fisher, R. A. (1936). “The Use of Multiple Measurements in Taxonomic Problems”. Annals of Eugenics 7 (2): 179-188.
D1. Sharma U K, Schito M, Welte A, Rousseau C, Fitzgibbon J, Keele B, et al. Workshop summary: Novel biomarkers for HIV incidence assay development. AIDS research and human retroviruses. 2012; 28(6):532-9.
D2. Incidence Assay Critical Path Working G. More and better information to tackle HIV epidemics: towards improved HIV incidence assays. PLoS medicine. 2011; 8(6):e1001045.
D3. Rerks-Ngarm S, Pitisuttithum P, Nitayaphan S, Kaewkungwal J, Chiu J, Paris R, et al. Vaccination with ALVAC and AIDSVAX to prevent HIV-1 infection in Thailand. The New England journal of medicine. 2009; 361(23):2209-20.
D4. Cohen M S, Chen Y Q, McCauley M, Gamble T, Hosseinipour M C, Kumarasamy N, et al. Prevention of HIV-1 infection with early antiretroviral therapy. The New England journal of medicine. 2011; 365(6):493-505.
D5. Baeten J M, Donnell D, Ndase P, Mugo N R, Campbell J D, Wangisi J, et al. Antiretroviral prophylaxis for HIV prevention in heterosexual men and women. The New England journal of medicine. 2012; 367(5):399-410.
D6. Kassanjee R, Pilcher C D, Keating S M, Facente S N, McKinney E, Price M A, et al. Independent assessment of candidate HIV incidence assays on specimens in the CEPHIA repository. Aids. 2014; 28(16):2439-49.
D7. Longosz A F, Morrison C S, Chen P L, Arts E, Nankya I, Salata R A, et al. Immune responses in Ugandan women infected with subtypes A and D HIV using the BED capture immunoassay and an antibody avidity assay. J Acquir Immune Defic Syndr. 2014; 65(4):390-6.
D8. Longosz A F, Serwadda D, Nalugoda F, Kigozi G, Franco V, Gray R H, et al. Impact of HIV subtype on performance of the limiting antigen-avidity enzyme immunoassay, the bio-rad avidity assay, and the BED capture immunoassay in Rakai, Uganda. AIDS research and human retroviruses. 2014; 30(4):339-44.
D9. Mullis C E, Munshaw S, Grabowski M K, Eshleman S H, Serwadda D, Brookmeyer R, et al. Differential specificity of HIV incidence assays in HIV subtypes A and D-infected individuals from Rakai, Uganda. AIDS research and human retroviruses. 2013; 29(8):1146-50.
D10. Kassanjee R, Pilcher C D, Busch M P, Murphy G, Facente S N, Keating S M, et al. Viral load criteria and threshold optimization to improve HIV incidence assay characteristics. Aids. 2016; 30(15):2361-71.
D11. Murphy G, Pilcher C D, Keating S M, Kassanjee R, Facente S N, Welte A, et al. Moving towards a reliable HIV incidence test—current status, resources available, future directions and challenges ahead. Epidemiology and infection. 2016:1-17.
D12. Morrison C H R, Mack N, Seepolmuang P, Averill M, Taylor J, Osborn J, Dailey P, Parkin N, Ongarello S, Mastro T D. HIV Research for Prevention (HIVR4P) Conference. Chicago, Ill.
D13. UNAIDS. TECHNICAL UPDATE ON HIV INCIDENCE ASSAYS FOR SURVEILLANCE AND MONITORING PURPOSES.
D14. Yates N L, Stacey A R, Nolen T L, Vandergrift N A, Moody M A, Montefiori D C, et al. HIV-1 gp41 envelope IgA is frequently elicited after transmission but has an initial short response half-life. Mucosal immunology. 2013; 6(4):692-703.
D15. Tomaras G D, Yates N L, Liu P, Qin L, Fouda G G, Chavez L L, et al. Initial B-cell responses to transmitted human immunodeficiency virus type 1: virion-binding immunoglobulin M (IgM) and IgG antibodies followed by plasma anti-gp41 antibodies with ineffective control of initial viremia. Journal of virology. 2008; 82(24):12449-63.
D16. Yates N L, Lucas J T, Nolen T L, Vandergrift N A, Soderberg K A, Seaton K E, et al. Multiple HIV-1-specific IgG3 responses decline during acute HIV-1: implications for detection of incident HIV infection. Aids. 2011; 25(17):2089-97.
D17. Wilson K M, Johnson E I, Croom H A, Richards K M, Doughty L, Cunningham P H, et al. Incidence immunoassay for distinguishing recent from established HIV-1 infection in therapy-naive populations. Aids. 2004; 18(17):2253-9.
D18. Curtis K A, Kennedy M S, and Owen S M. Longitudinal analysis of HIV-1-specific antibody responses. AIDS research and human retroviruses. 2014; 30(11):1099-105.
D19. Liu P, Overman R G, Yates N L, Alam S M, Vandergrift N, Chen Y, et al. Dynamic antibody specificities and virion concentrations in circulating immune complexes in acute to chronic HIV-1 infection. Journal of virology. 2011; 85(21):11196-207.
D20. Curtis K A, Kennedy M S, Charurat M, Nasidi A, Delaney K, Spira T J, et al. Development and characterization of a bead-based, multiplex assay for estimation of recent HIV type 1 infection. AIDS research and human retroviruses. 2012; 28(2):188-97.
D21. Mastro T D, Kim A A, Hallett T, Rehle T, Welte A, Laeyendecker O, et al. Estimating HIV Incidence in Populations Using Tests for Recent Infection: Issues, Challenges and the Way Forward. J HIV AIDS Surveill Epidemiol. 2010; 2(1):1-14.
D22. Busch M P, Pilcher C D, Mastro T D, Kaldor J, Vercauteren G, Rodriguez W, et al. Beyond detuning: 10 years of progress and new challenges in the development and application of assays for HIV incidence estimation. Aids. 2010; 24(18):2763-71.
D23. Janssen R S, Satten G A, Stramer S L, Rawal B D, O'Brien T R, Weiblen B J, et al. New testing strategy to detect early HIV-1 infection for use in incidence estimates and for clinical and prevention purposes. JAMA. 1998; 280(1):42-8.
D24. Parekh B S, Kennedy M S, Dobbs T, Pau C P, Byers R, Green T, et al. Quantitative detection of increasing HIV type 1 antibodies after seroconversion: a simple assay for detecting recent HIV infection and estimating incidence. AIDS research and human retroviruses. 2002; 18(4):295-307.
D25. Duong Y T, Qiu M, De A K, Jackson K, Dobbs T, Kim A A, et al. Detection of recent HIV-1 infection using a new limiting-antigen avidity assay: potential for HIV-1 incidence estimates and avidity maturation studies. PloS one. 2012; 7(3):e33328.
D26. Curtis K A, Hanson D L, Kennedy M S, and Owen S M. Evaluation of a multiplex assay for estimation of HIV-1 incidence. PloS one. 2013; 8(5):e64201.
D27. Cohen M S, Gay C L, Busch M P, and Hecht F M. The detection of acute HIV infection. The Journal of infectious diseases. 2010; 202 Suppl 2:S270-7.
D28. Counihan N A, and Anderson D A. Specific IgA Enhances the Transcytosis and Excretion of Hepatitis A Virus. Scientific reports. 2016; 6:21855.
D29. Santra S, Tomaras G D, Warner R, Nicely N I, Liao H X, Pollara J, et al. Human Non-neutralizing HIV-1 Envelope Monoclonal Antibodies Limit the Number of Founder Viruses during SHIV Mucosal Infection in Rhesus Macaques. PLoS Pathog. 2015; 11(8):e1005042.
D30. Liao H X, Bonsignori M, Alam S M, McLellan J S, Tomaras G D, Moody M A, et al. Vaccine induction of antibodies against a structurally heterogeneous site of immune pressure within HIV-1 envelope protein variable regions 1 and 2. Immunity. 2013; 38(1):176-86.
D31. Montefiori D C, Karnasuta C, Huang Y, Ahmed H, Gilbert P, de Souza M S, et al. Magnitude and breadth of the neutralizing antibody response in the RV144 and Vax003 HIV-1 vaccine efficacy trials. The Journal of infectious diseases. 2012; 206(3):431-41.
D32. Zhang R, Alam S M, Yu J S, Scearce R, Lockwood B, Hwang K K, et al. Novel Monoclonal Antibodies for Studies of Human and Rhesus Macaque Secretory Component and Human J-Chain. Monoclon Antib Immunodiagn Immunother. 2016; 35(4):217-26.
D33. Katinger H. Human monoclonal antibodies for passive immunotherapy of HIV-1. Antibiotics and chemotherapy. 1994; 46:25-37.
D34. Hwang K K, Trama A M, Kozink D M, Chen X, Wiehe K, Cooper A J, et al. IGHV1-69 B cell chronic lymphocytic leukemia antibodies cross-react with HIV-1 and hepatitis C virus antigens as well as intestinal commensal bacteria. PloS one. 2014; 9(3):e90725.
D35. Tabachnick B, and Fidell L S. Using Multivariate Statistics. New York, N.Y.: HarperCollins College Publishers; 1996.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A method for classifying HIV-1 status of a sample from a subject as recent or long standing, the method comprising:
a) Measuring in the sample binding of IgG3 antibodies to Clade C gp140 envelope (measurement X1);
b) Measuring in the sample the avidity of IgG4 antibodies binding to T/F Clade C gp140 envelope (measurement X2);
c) Measuring in the sample the avidity of IgG4 antibodies binding to Clade B gp140 envelope (measurement X3);
d) Measuring in the sample the avidity of IgG to gp41 immunodomninant domain (ID) epitope (measurement X4), wherein the avidity of IgG gp41 ID epitope is determined as difference between IgG Citrate gp41 ID epitope and IgG PBS gp41 ID epitope;
e) Using measurements X1, X2, X3 and X4 to determine recent (R) or longstanding (L) discriminant value (DVⱼ is used interchangeably Cⱼ), wherein “j” is recent (R) or longstanding (L) class, wherein cut off period for R is nine months, and wherein
i. DV_{R}=co_{R}+C_{1R}X1+c_{2R}X2+c_{3R}X3+c_{4R}X4, wherein R classification coefficients are as follows: Table 40 or Table 41, and
ii. DV_{L}=co_{L}+c_{1L}X1+c_{2L}X2+c_{3L}X3+c_{4L}X4, wherein L classification coefficients are as follows: Table 40 or Table 41; and
f) Classifying a sample as being a recent infection or a long-standing infection, wherein if DV_{R} is larger than DV_{L}, the sample is classified as Recent, or wherein if DV_{L} is larger than DV_{R}, the sample is classified as Longstanding.

2. The method of claim 1, wherein Clade C gp140 envelope for measurement X1 is any one Clade C envelope in FIG. 62.

3. The method of claim 2, wherein Clade C gp140 envelope for measurement X1 is BF1266 gp140 (FIG. 59), comprising LANL database accessioning number HM215360.

4. The method of claim 1, wherein T/F Clade C gp140 envelope for measurement X2 is any one of the T/F clade C envelopes in FIG. 62.

5. The method of claim 4, wherein the T/F clade C gp140 for measurement X2 is T/F CH505 gp140 (FIG. 59), comprising LANL database accessioning number KC247557.

6. The method of claim 1, wherein the clade B gp140 envelope for measurement X3 is any one of Clade B envelopes in FIG. 62, comprising LANL database accessioning number AY835447, AY835441, or AY835451.

7. The method of claim 6, wherein the clade B gp140 envelope for measurement X3 is SC42261 gp140 in FIG. 59, comprising LANL database accessioning number AY835441.

8. The method of claim 1, wherein the gp41 ID epitope for measurement X4 is any one listed in FIG. 62.

9. The method of claim 8, wherein the gp41 ID epitope for measurement X4 is B.con03 B.con03 ID/(Bio-GGG-BC.con03 ID), comprising amino acid sequence: Bio-GGG-KQLQARVLAVERYLKDQQLLGIWGCSGKLICTTAV.

10. The method of claim 1, wherein the Clade C gp140 envelope for measurement X1 is BF1266 gp140 (FIG. 59), the T/C clade C gp140 for measurement X2 is T/F CH505 gp140 (FIG. 59), the clade B gp140 envelope for measurement X3 is SC42261 gp140 (FIG. 59) and the gp41 ID epitope for measurement X4 is B.con03 ID/(Bio-GGG-BC.con03 ID).

11. The method of claim 1 or 10, wherein
i. For DV_{R}=co_{R}+c_{1R}X1+c_{2R}X2+c_{3R}X3+c_{4R}X4, the R classification coefficients are as follows: Table 40, and
ii. For DV_{L}=co_{L}+c_{1L}X1+c_{2L}X2+c_{3L}X3+c_{4L}X4, the L classification coefficients are as follows: Table 40; and
iii. wherein if DV_{R} is larger than DV_{L}, the sample is classified as Recent, or wherein if DV_{L} is larger than DV_{R} the sample is classified as Longstanding.

12. A method for classifying HIV-1 status of a sample from a subject as recent or long standing, the method comprising:
Detecting and quantifying the formation of antibody-antigen complexes, wherein the antibody-antigen complexes comprise:
i. avidity of IgG3 antibodies to p66 (measurement p66X1),
ii. avidity of IgM antibodies binding to gp41 (measurement gp41X2),
iii. avidity of IgG4 antibodies binding to envelope WITO4160 gp140 (measurement WITOX3),
iii. binding of IgM to gp41 immunodomninant domain (IG) (measurement gp41X4),
Measuring in the sample the avidity of IgG3 antibodies to p66 (measurement p66X1);
Measuring in the sample the avidity of IgM antibodies binding to gp41 (measurement gp41X2);
Measuring in the sample the avidity of IgG4 antibodies binding to envelope WITO4160 gp140 (measurement WITOX3), comprising LANL database accessioning number AY835451;
Measuring in the sample binding of IgM to gp41 (measurement gp41X4); and
Using measurements X1, X2, X3 and X4 to determine recent (R) or longstanding (L) discriminant value (DVⱼ is used interchangeably Cⱼ), wherein “j” is recent (R) or longstanding (L) class, wherein cut off period for R is nine months, and wherein
i. DV_{R}=co_{R}+c_{1R}X1+c_{2R}X2+c_{3R}X3+c_{4R}X4, wherein R classification coefficients are as follows: Table 41, and
ii. DV_{L}=co_{L}+c_{1L}X1+c_{2L}X2+c_{3L}X3+c_{4L}X4, wherein L classification coefficients are as follows: Table 41; and
Classifying the HIV status in the sample as being a recent infection or a long-standing infection, wherein if DV_{R} is larger than DV_{L}, the sample is classified as Recent, or wherein if DV_{L} is larger than DV_{R} the sample is classified as Longstanding.

13. The method of claim 12, wherein gp41 comprises Clade B (MN) gp41 protein.

14. A kit comprising a selection of HIV-1 antigens to determine measurements X1, X2, X3 and X4 of claims 1-12 or the measurements of claim 13 in a biological sample.

15. The kit of claim 14, wherein the HIV-1 antigens are immobilized on a solid support.

16. The kit of claim 14, wherein the HIV-1 antigens are conjugated to beads.

17. The kit of claim 14 further comprising instructions on how to carry out the steps of any one of the above claim 1-12 or 13.

18. The kit of claim 17, wherein the instructions include instructions on determining DV_{R} and DV_{L}, and the

[symbol]

19. The kit of any one of claims 14-18, wherein the sample is plasma, serum, finger stick blood, dried blood spot (DBS), whole blood, saliva, urine, mucosal fluid, or any other suitable biological sample.

20. The method of any one of claims 1-13, wherein the sample is plasma, serum, finger stick blood, dried blood spot (DBS), whole blood, saliva, urine, mucosal fluid, or any other suitable biological sample.
